(19) United States
(12) Patent Application Publication
(10) Pub. No.: US 2021/0214794 A1
(43) Pub. Date: Jul. 15, 2021

(54) BIOMARKER METHODS FOR TREATMENT OF ATOPIC DISEASE BY IMMUNOTHERAPY

(71) Applicant: ALD-ABELLÓ A/S, Hörsholm (DK)

(72) Inventors: Ilka Hoof, Frederiksberg (DK); Peter Sejer Andersen, Vanløse (DK); Thomas Stranzl, Farum (DK); Stephanie Bramd, København N (DK)

(73) Assignee: ALD-ABELLÓ A/S, Hörsholm (DK)

(21) Appl. No.: 17/056,035

(22) PCT Filed: May 22, 2019
(86) PCT No.: PCT/EP2019/063213

§ 371 Date: Nov. 17, 2020

(30) Foreign Application Priority Data
May 22, 2018 (EP) 18173551.5

(51) Int. Cl. C12Q 1/6883; A61K 39/35; A61P 37/08

(52) U.S. Cl. CPC C12Q 1/6883; A61K 39/35; C12Q 2600/156; C12Q 2600/106; A61P 37/08

(57) ABSTRACT

The invention provides methods related to the detection of specific biomarkers in combination with use of immunotherapy products for treatment of atopic disease. More specifically, biomarkers useful for selecting subjects for treating, preventing, or reducing risk of asthma by use of immunotherapy, e.g. allergen-specific immunotherapy. Further, the invention provides immunotherapy products for use in methods involving the detection of one or more biomarkers, as well as kits of parts for detection of biomarkers related to asthma.

### FIELD OF THE INVENTION

The invention provides methods related to the detection of specific biomarkers in combination with use of immunotherapy products for treatment of atopic disease. More specifically, biomarkers useful for selecting subjects for treating, preventing, or reducing risk of asthma by use of immunotherapy, e.g. allergen-specific immunotherapy. Further, the invention provides immunotherapy products for use in methods involving the detection of one or more biomarkers, as well as kits of parts for detection of biomarkers related to asthma.

### BACKGROUND OF THE INVENTION

Asthma is a heterogeneous condition comprising a wide range of clinical phenotypes having airway inflammation and bronchial hyper-responsiveness as common features.

In some subjects, asthma is associated with atopy, e.g. is an atopic disease, where airborne allergens, like house dust mites or pollen, may be an important trigger of allergic asthma. Early-onset asthma is often associated with allergic asthma, which usually starts in childhood and often is accompanied or preceded by other atopic diseases, like allergic rhinitis, atopic dermatitis and/or food allergy. The proportion of asthma that is allergic varies from about 30 to 80% in children and from 30 to 60% in adults (EAACI guidelines 2017). The allergic march refers to an often observed progression pattern of atopic disorders, wherein the initial development of atopic dermatitis and concomitant sensitization to food and aeroallergens in early childhood is progressing to development of asthma and allergic rhinitis in later childhood or adult life.

Asthma may also be non-related to atopy and instead being related other factors like rhino sinusitis and/or nasal polyposis, respiratory virus infections, exposure to tobacco smoking in early childhood, or associated with obesity. Later-onset asthma is often not related to atopy. The above-mentioned asthma phenotypes can be divided further into endotypes linked to the underlying molecular, functional, or pathological mechanisms of disease.

Asthma heritability is estimated to be 70-90%, but only a limited number of susceptibility loci have been verified in genome-wide association studies (GWAS). Articles by Ober and Yao 2011 and Moffat et al. 2010 discuss various GWAS studies, single nucleotide polymorphisms (SNPs) and the complexity of using single nucleotide polymorphisms for prediction of asthma disease. Further, patent applications WO2008155396, WO2009140699 and WO2018018004 disclose a number of SNPs related to asthma, including SNPs located at the 17q12-21 loci, the 6q21.31, 6q21.32-33, the 1q31, the 7q22.3, the 5q22.1, the 9q21.2 and 10q21.3 loci.

Several GWAS and non-GWAS studies have demonstrated a strong genetic linkage of single nucleotide polymorphisms (SNPs) in the chromosomal region 17q12-21 with asthma in populations of diverse ethnic backgrounds (Stein et al. 2018 and references cited therein). In particular, one SNP (r57216389 (SEQ ID NO: 10)) in the 17q21 region has showed the strongest association with childhood and adult asthma in different ethnic populations (Moffatt et al. 2007, WO2008155396, Galanter et al. 2008 and Madore et al. 2008).

A number of studies have reported gene-environment interactions, where a specific environmental factor was found to influence the risk of asthma in subjects with SNPs in the 17q12-21 region. Further, Loss et al. 2016 reported increased risk of early wheeze for school-age asthma for subjects having the 17q21 SNPs r58076131 (SEQ ID NO: 12), r57216389 (SEQ ID NO: 10) and r52290400, but that exposure to animal sheds significantly reduced the occurrence of wheezing in the first year of life for subjects having the r58076131 (SEQ ID NO: 12) SNP. The authors suggest that the exposure to animal sheds may reduce genetically determined asthma risk by promoting the capability of coping with early viral infections, which often are associated with non-atopy related asthma. Blelic et al. 2013 found that some SNPs in the 17q12-21 region decrease the risk for asthma, but only if furry pets were present in the home during the first year of life. In other children carrying other SNPs in the 17q12-21 region, pet ownership increased the risk of hospital admission. Stokholm et al. 2017 reported that cat and/or dog exposure from birth was associated with a lower prevalence of asthma among children with the r57216389 (SEQ ID NO: 10) high-risk TT genotype.

Despite these gene-environment interactions, another study reported by Bisgaard et al. concluded that the r57216389 (SEQ ID NO: 10) SNP did not confer risk of eczema, rhinitis, or allergic sensitization, and that the 17q21 locus was an important genetic determinant of non-atopic (i.e. non-allergic) asthma in children (Bisgaard et al. 2009). This finding is supported in a review article by Ober and Yao 2012, which mentions that the variation at the 17q21 locus seems to be specific to asthma and not to IgE levels and that there is compelling evidence that this important locus is a primary asthma-susceptibility locus that acts independently of atopic, or IgE-mediated pathways (Ober and Yao 2012, Moffat et al. 2010).

When it comes to the treatment of asthma, there is a number of options. The main treatment for asthma is using control medications each day, preferably for reducing inflammation in the airways to reduce the risk of having an asthma attack. Current control medications focus on alleviating symptoms by using inhaled corticosteroids (ICS) such as Beclomethasone dipropionate or budesonide, and in more severe cases of asthma the ICS are used in combination with long-acting β2-adrenergic agonists (LABA), e.g. Salmeterol (Tamm et al. 2012). Further, to treat such cases of asthma, the dose of ICS is often increased. If the control medications (ICS) do not work quickly enough to treat an asthma attack, rescue medications may be an option to open up the airways rapidly, e.g. short-acting β2-adrenergic agonists (SABA) like salbuterol (albuterol).

However, these treatments have a number of disadvantages:
There are concerns about the systemic effects of ICS, particularly in children. Long-term use of ICS can cause impaired growth, decreased bone mineral density, skin thinning and bruising, and cataracts (Dahl et al. 2006). Thus, there is a considerable medical need for therapeutic interventions that can reduce the long-term use of ICS, particularly since even patients with relatively mild disease well controlled by properly managed ICS usage would be concerned about the potential negative effects of long-term usage of ICS.
Additionally, the use of ICS in combination β2-adrenergic agonist is not sufficient in controlling asthma in subject suffering from severe asthma or refractory asthma, where the patients often need emergency-room visits or hospitalization and treatment with systemic corticosteroids (such as or oral corticosteroids (OCS)) in order to treat the disease. Further, in chronic asthmatics, long term airway remodeling is also observed (Possa et al. 2013). Airway remodeling causes fixed airway obstruction and is associated with poorer clinical outcome. Early diagnosis and prevention of airway remodeling has the potential to decrease disease severity, to improve control, and to prevent disease expression.
Asthma patients whose symptoms can usually be rapidly relieved by inhaled ICS and/or use of a β2-adrenergic agonist, may still be at risk of experiencing a severe asthma exacerbation, or even death due to asthma. Therefore it is of high importance to identify treatments and/or methods of treatment which are capable of reducing the risk of more severe asthma exacerbations.

Alternative treatments of asthma have been explored within the last decade, including antibody therapy (biologics), such as anti-IgE (Omalizumab), anti-IL4Rα (Dupilumab), anti-IL13 (Tralokinumab), anti-IL5 (Mepolizumab, Reslizumab) and anti-IL5 receptor (Benralizumab) antibodies (see Katial et al. 2017); and allergen-specific immunotherapy (AIT). According to EAACI guidelines (EAACI guidelines 2017), AIT can be recommended for treating patients having mild and moderate allergic asthma, provided that the allergic asthma is adequately controlled by pharmacotherapy (e.g. asthma control medication). With respect to the antibody therapies, these are often only recommended in patients being characterized by having high blood eosinophil counts in order to target the treatment for patients with Th2-high inflammatory endotype and/or having high serum IgE levels.

To select the most beneficial treatment for a given patient there is a need for biomarkers, which are useful in predicting whether a given therapy provides efficacy in treating asthma and thus to provide precision medicine for asthma treatment.

The basic principle of precision medicine is to match each patient with the treatment that will work best for him or her. In order to enable for the use of precision medicine in patients suffering from asthma, a validated set of biomarkers are needed that could allow for a precise endotyping of asthma patients and thereby provide guides for the selection of the most relevant treatment for the patient. In line with the principles of precision medicine, recently approved biologics for treatment of patients with severe asthma are restricted to patients with high blood eosinophil counts in order to enrich the target population for the Th2-high inflammatory endotype. However, considering the complexity of disease endotypes in asthma, more biomarkers are needed to reach the full potential of precision medicine (see for example Katial et al. 2017, Chiappori et al. 2015 and Zissler et a. 2016).

Concerning AIT, there is no consensus on candidate surrogate biomarkers of efficacy or biomarker combinations that would be prognostic, predictive and/or surrogate of the clinical response to AIT (FDA 2008 and Shamji et al. 2017). Common biomarkers used during clinical development of AIT vaccines include monitoring changes in levels of allergen-specific IgE and IgG4 antibodies. Although these biomarkers give very consistent results across clinical trials, no association to clinical effect has been demonstrated (Shamji et al. 2017).

Thus, there is a need of biomarkers that can guide appropriate asthma treatment for subjects already diagnosed with asthma. In addition there is a need for biomarkers that can guide appropriate asthma prevention therapies for subjects which are at risk of developing asthma, for example because of the allergic march phenomena.

WO 2010/102387 A1 discloses commercial kits for detection of SNPs in respect of genotyping a number polymorphisms, including rs907092, rs9303277, rs2305480, rs2290400 and rs7216389 at the 17q12-21 locus and information about detection of SNPs by the Infinium Global Screening Array-24 can be found on documents published on the the website:URL:https://www.illumina.com/content/dam/illumina-marketing/documents/products/datasheets/infinium-commercial-gsa-data-sheet-370-2016-016

WO 2009/117547 A2 discloses a method for predicting response to anti-TNF antibody treatment (i.e. immunotherapy) in a patient with severe or persistent asthma based on the allelic identity at particular SNPs in the TNFRSF1 genes.

WO 2017/121883 discloses a method for predicting responsiveness of a patient to immunotherapy with a house dust mite allergen based on the level of IL-10 RNA in a sample from the patient.

Stein et al. 2018 relates to SNPs located in the chromosomal region 17q12-21, which seems to be associated with risk of developing asthma.

### SUMMARY OF THE INVENTION

The present invention provides biomarkers useful for predicting beneficial effect of treatment of atopic disease with immunotherapy products, such as for example the treatment of asthma with allergen-specific immunotherapy. More specifically, single nucleotide polymorphisms and/or Th2 markers are provided which are useful for predicting effect and/or selecting patients for treatment with immunotherapy products in subjects suffering from atopic disease, e.g. asthma.

The inventors of the present invention surprisingly found that the presence of SNPs of the 17q12-21 region and/or a high level of specific Th2 markers (eosinophil count, eosinophil cationic protein (ECP), tryptase and periostin) are correlated with efficacy or beneficial response of treatment of atopic disease (e.g. asthma), when a subject is treated with immunotherapy products (allergen-specific immunotherapy). This is for example demonstrated in Example 1, which describes a study wherein the treatment with allergen-specific immunotherapy was able to significantly reduce the risk of moderate to severe asthma exacerbations in subjects having one or more of SNPs of the 17q12-21 region and suffering from allergic asthma, or in subjects having a high level of one or more Th2 markers selected from the group consisting of blood eosinophil count, serum eosinophil cationic protein (ECP), serum tryptase and serum periostin. This is a particular advantage, since even patients which are usually able to control asthma symptoms by use of home-medication (such as ICS), are still at significant risk of experiencing moderate and/or severe asthma exacerbations, which require hospitalization and/or emergency room visit, and which may have a fatal outcome. Further, the selection of a group of patients suffering from severe asthma not well controlled by ICS in combination with SABA, LABA or LAMA, but having benefit of treatment with for example allergen-specific immunotherapy, may help to reduce costs related to hospitalization or alternative treatments available for such patients.

The biomarkers of the invention have the advantage that they can be measured and used for prediction of a beneficial effect of treatment prior to initiating treatment with immunotherapy products, and can therefore facilitate the use of immunotherapy products for treatment of a subject, e.g. be useful in selection of patients for treatment with such products.

Further, the detection of one or more SNPs of the 17q12-21 region has the advantage compared to other biomarkers that genotyping can be performed relatively easily, at low cost without the need for invasive methods.

Further, the use of genotyping or SNP analysis has the advantage that the testing can be performed at an early stage, before the manifestation of one or more atopic diseases (e.g. asthma), or before the state of atopy has progressed into more severe states of atopic disease, such as severe asthma. For example, a child having one or more SNPs of the 17q12-21 region, and having symptoms of atopic dermatitis and/or sensitization to one or more allergens, could be treated to prevent progression into asthma. Or a child having one or more SNPs of the 17q12-21 region, and having allergic rhinitis could be treated to prevent progression into asthma. Similarly, an adult patient having one or more SNPs of the 17q12-21 region and being diagnosed with mild asthma could be treated to prevent progression into severe asthma, and therapy reduce the need for use of corticosteroids and/or SABA, or other asthma medications.

One aspect according to the invention is a method for selecting a subject for treatment with an immunotherapy product (e.g. allergen-specific immunotherapy) for treatment of an atopic disease, e.g. asthma and/or predicting response to treatment with an immunotherapy product for treatment of an atopic disease, e.g. asthma comprising:
a) Detecting whether a biological sample obtained from said subject has one or more of:
   i. A single nucleotide polymorphism (SNP) selected from the group consisting of the single nucleic acid polymorphisms located in the chromosome 17q12-21 region and/or;
   ii. A SNP in linkage disequilibrium with one or more SNPs as defined in i) above;
b) Selecting said subject for treatment with an immunotherapy product and/or predicting a beneficial response to treatment with an immunotherapy product in said subject, if one or more SNPs of step a) are detected.

In some examples of the aspect above, the method further comprises a treatment step c) wherein an immunotherapy product is administered to a subject that is either selected for treatment according to step b) and/or predicted to have a beneficial response to treatment according to step b).

In some examples of the aspects of the invention, a method involves detecting whether the sample from said subject has one or more SNPs of Table 1, preferably SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10), or a SNP in linkage disequilibrium with one or more of the SNPs, or a complementary SNP thereof. Preferably, the subject is a homozygote with respect to one or more of the SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10) or a SNP in linkage disequilibrium with one or more of the SNPs, or a complementary SNP thereof. In some examples of the invention, a subject is selected from treatment and/or a beneficial response is predicted in the subject if is detected that the subject is a homozygote with respect to one or more of the SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10) or a SNP in linkage disequilibrium with one or more of the SNPs, or a complementary SNP thereof. In some examples, the subject is a homozygote with respect to the one or more SNP's detected in a biological sample from the subject.

In some examples of the aspects of the invention, the immunotherapy product is for allergen specific immunotherapy, e.g. comprising one or more house dust mite allergens, or comprising one or more grass pollen allergens.

Another aspect of the invention provides a method of treating, preventing, delaying onset, or reducing risk of an atopic disease, such as asthma, in a subject in need thereof, said method comprising:
a) Obtaining and/or providing a biological sample comprising nucleic acids from said subject; and
b) Detecting whether the nucleic acids obtained from said subject has one or more of:
   i. A single nucleotide polymorphisms (SNPs) selected from the group consisting of the single nucleic acid polymorphisms located in the chromosome 17q12-21 region and/or;
   ii. A SNP in linkage disequilibrium with one or more SNPs as defined in i) above;
c) Identifying or selecting said subject for treatment with immunotherapy if one or more SNPs of section b) are identified in the nucleic acids comprised in said biological sample; and
d) Administering an immunotherapy product (e.g. for allergen specific immunotherapy) to said subject identified or selected according to step c).

In some examples of the aspects of the invention, the immunotherapy product is for allergen specific immunotherapy, e.g. comprising one or more house dust mite allergens, or comprising one or more grass pollen allergens.

Another aspect of the invention provides an immunotherapy product for use in a method of treating, preventing or reducing risk of one or more atopic diseases, such as asthma, where the treating method is as defined in the aspects above.

A further aspect of the invention is the use of an allergen and/or antibody in the manufacture of a medicament for the treatment of atopic disease such as asthma, wherein the medicament for the treatment of atopic disease is an immunotherapy product for use in a treatment method comprising a step of detecting wherein a SNP located in the chromosome 17q12-21 region as described above.

Still further aspect of the invention are a kit of parts comprising means and instructions for use in methods as defined herein. Still further aspects of the invention is the use of the SNPs described herein for predicting responsiveness to treatment, and the use of the SNPs as described herein for selecting patients for treatment.

Other aspects of the invention relate to the use of Th2 biomarkers, optionally in combination with the use of SNPs of the 17q12-21 region as described in aspects above. Thus one aspect of the invention provides a method for selecting a subject for treatment with an immunotherapy product for treatment of an atopic disease, e.g. asthma and/or predicting response to treatment with an immunotherapy product (e.g. for allergen-specific immunotherapy) for treatment of an atopic disease, e.g. asthma, comprising:
a) Quantifying the level of one or more Th2 markers selected from the group consisting of eosinophil count, eosinophil cationic protein (ECP), tryptase, periostin and total IgE;
b) Selecting said subject for treatment with an immunotherapy product and/or predicting a beneficial response to treatment with an immunotherapy product in said subject, if the level of one or more markers of step a) is increased or above a reference level.

Such a method may further comprise a treatment step c) wherein an immunotherapy product is administered to a subject that is either selected for treatment according to step b) and/or predicted to have a beneficial response to treatment according to step b).

Other aspects of the invention provide a method of treating, preventing, delaying onset, or reducing risk of an atopic disease, such as asthma, in a subject in need thereof, said method comprising:
a) Obtaining and/or providing a biological sample from said subject; and
b) Quantifying the level of one or more Th2 markers selected from the group consisting of eosinophils, eosinophil cationic protein (ECP), tryptase, periostin, and total IgE;
c) Identifying or selecting said subject for treatment with an immunotherapy product and/or predicting a beneficial response to treatment with an immunotherapy product (e.g. for allergen-specific immunotherapy) in said subject, if the level of one or more markers of step a) is increased or above a reference level; and
d) Administering an immunotherapy product to said subject identified or selected according to step c).

Another aspect of the invention provides an immunotherapy product for use in a method of treating, preventing or reducing risk of one or more atopic diseases, such as asthma, where the treating method is as defined in the aspects using Th2 markers above.

A further aspect of the invention is the use of an allergen and/or antibody in the manufacture of a medicament for the treatment of atopic disease such as asthma, wherein the medicament for the treatment of atopic disease is an immunotherapy product for use in a treatment method comprising a step of counting, quantifying the level of, or measuring the concentration of the Th2 markers mentioned above.

Still further aspect of the invention are a kit of parts comprising means and instructions for use in methods as defined herein. Still further aspects of the invention is the use of the Th2 markers described herein for predicting responsiveness to treatment, and the use of the Th2 markers as described herein for selecting patients for treatment.

### DESCRIPTION OF DRAWINGS

FIG. 1: Proportion of trial participants with a certain rs7216389 (SEQ ID NO: 10) genotype who experienced an asthma exacerbation in the three treatment groups of Example 1. Error bars indicate the 95% confidence interval. The bars show the proportion of each subpopulation (defined by SNP genotype and treatment group) who experienced an asthma exacerbation.

FIG. 2: Proportion of trial participants with “high” and “normal” baseline levels of ECP who experienced an asthma exacerbation in the three treatment groups of Example 1. Error bars indicate the 95% confidence interval. The bars show the proportion of each subpopulation (defined by ECP level and treatment group) who experienced an asthma exacerbation.

FIG. 3: Venn diagram showing the number of subjects defined as “Th2 high” based on one single biomarker or the combination of 2, 3 or 4 of the biomarkers.

FIG. 4: Barplots showing the proportion of trial participants in “Th2 high” and “Th2 normal” groups who experienced an asthma exacerbation in the three treatment groups of Example 1. Th2 levels were defined based on a combination of A) eosinophil count and tryptase levels and B) ECP, tryptase and periostin. A subject was defined as “Th2 high” if at least one measured value of the Th2 markers considered in the combination was above a threshold marking the 20% highest values for the given biomarker in the trial population. Error bars indicate the 95% confidence interval. The bars show the proportion of each subpopulation (defined by Th2 level and treatment group) who experienced an asthma exacerbation.

### DETAILED DISCLOSURE OF THE INVENTION

### Definitions

“Allele” as used herein refers to a variant of a SNP (a position wherein variation in the type of nucleotide is found in the DNA in a given population). When there are two, three or four alternative nucleotides at a polymorphic site, each nucleotide sequence is referred to as an allele or “polymorphic variant” or “nucleic acid variant.” A “reference allele” is an allele that is found in the majority of a reference population, e.g. an allele found in the majority of a population of healthy subjects. “Effect allele” when used herein is an allele that correlates with an increased effect on reducing risk of, or treatment for, a disease or disorder (e.g. an atopic disorder such as asthma) or is associated with an odds ratio or relative effect of >1. The “effect allele” may be the minor allele or major allele. “Effect genotype” when used herein is the genotype of a polymorphism that correlates with an increased effect, e.g. on reducing risk of, or treatment for, an atopic disease or disorder (e.g. asthma), or reducing the occurrence of, or ameliorate, symptoms or signs of an atopic disease as defined herein. “Risk allele” when used herein is an allele that correlates with an increased risk for a disease or disorder (e.g. an atopic disorder such an asthma) or is associated with an odds ratio or relative risk of >1. The “risk allele” may be the minor allele or major allele. “Risk genotype” when used herein is the genotype of a polymorphism that correlates with an increased risk for a atopic disease, (e.g. asthma), diagnostic measures such as symptoms and signs of an atopic disease described herein. Where two polymorphic variants exist, the polymorphic variant represented in a majority of samples from a population is referred to as a “prevalent allele,” or “major allele,” and the polymorphic variant that is less prevalent in the population is referred to as an “uncommon allele” or “minor allele.” An individual who carries the same allele on the two versions of a particular chromosome is “homozygous” with respect to the polymorphism or allele. An individual who carries two different alleles on the two versions of a particular chromosome is “heterozygous” with respect to the polymorphism. The specific nucleotide in an allele is dependent on the strand used to extract the data from the genotyping platform, since nucleotides of the forward strand is complementary to the nucleotides of the backwards strand.

The term “allergen” refers to an antigen or agent which elicits, induces, stimulates, or enhances an immune response by a cell of the immune system of an exposed subject such as an animal (e.g., a human being). An antigen is an allergen when the specific immune response is the development of enhanced sensitivity or a hypersensitivity to the antigen, but the antigen itself is not typically innately harmful. An allergen is therefore a particular type of antigen that can cause development of enhanced or increased sensitivity or hypersensitivity in a subject. For example, an allergen can elicit production of IgE antibodies in predisposed subjects.

The term “allergic response” is intended to refer to the hypersensitive immune reaction to a normally innocuous environmental substance known as an allergen. A common mechanism of allergic reactions is the binding of IgE to the surface of mast cells and basophils, which may lead to symptoms or signs of atopic disease, such as asthma, allergic rhinitis and/or allergic conjunctivitis and other common allergic reactions.

The term “antibody” as used herein includes polyclonal and monoclonal antibodies of any type and from any species, as well as immunoglobulin fragments such as Fv, Fab, Fab′, F(ab′)2, or other antigen-binding antibody fragments, sequences or subsequences that interact with molecular specificity (e g., demonstrate specific binding) with a molecule, e.g. a protein.

The term “atopy” or “atopic disease” involves the sensitization to one or more allergens and/or the presence of detectable IgE in response to common environmental proteins or other allergens. The presence of atopy in an individual is associated with an increased risk of developing one or more of the atopic diseases—such as atopic dermatitis, allergic asthma, allergic rhinitis, allergic conjunctivitis or allergic rhinoconjunctivitis, food allergy, anaphylaxis, anaphylactic shock, allergic bronchopulmonary aspergillosis, urticaria, itch and hives. Atopy or atopic disease may or may not be accompanied by symptoms of allergy, such has clinical symptoms of allergy.

The terms “biomarker” and “marker” are used interchangeably herein to refer to a DNA, RNA, single nucleotide polymorphism (SNP), protein, carbohydrate, or glycolipid-based molecular marker, the expression or presence of which in a subject's or patient's sample can be detected by standard methods (or methods disclosed herein) and is for example useful for identifying the risk profile of a subject for a disease, disorder and condition and/or the likelihood of responsiveness or sensitivity of a mammalian subject to a treatment (e.g., a treatment with immunotherapy such as for example AIT). Expression of such a biomarker may be determined to be higher or lower in a sample obtained from a patient than a reference level (including, e.g., the median expression level of the biomarker in samples from a group/population of patients (e.g., asthma patients); the level of the biomarker in samples from a group/population of control individuals (e.g., healthy individuals); or the level in a sample previously obtained from the individual at a prior time).

The term “effective amount” refers to an amount of a drug effective to treat a disease or disorder in a subject or patient, such as a mammal, e.g., a human.

The term “genotype” refers to a description of the alleles of a gene contained in an individual or a sample. In the context of this invention, no distinction is made between the genotype of an individual and the genotype of a sample originating from the individual. Although typically a genotype is determined from samples of diploid cells, a genotype can be determined from a sample of haploid cells, such as a sperm cell.

“Genetic alteration” when used herein refers to a change from the wild-type or reference sequence of one or more nucleic acid molecules. Genetic alterations include without limitation, base pair substitutions, additions and deletions of at least one nucleotide from a nucleic acid molecule of known sequence.

“Haplotype” when used herein refers to a group of alleles on a single chromosome that are closely enough linked to be inherited usually as a unit.

“Linkage” denotes the tendency of genes, alleles, loci or genetic markers to be inherited together due to their location on a specific chromosome, and is measured by percent recombination (also called recombination fraction, or Θ) between the two alleles, genes, loci or genetic markers. The recombination fraction is often low if two loci are localized in physical proximity on a chromosome.

“Linkage disequilibrium” or “LD” when used herein refers to alleles at different loci that are not associated at random, i.e., not associated in proportion to their frequencies. If the alleles are in positive linkage disequilibrium, then the alleles occur together more often than expected assuming statistical independence. Conversely, if the alleles are in negative linkage disequilibrium, then the alleles occur together less often than expected assuming statistical independence. Linkage disequilibrium can be determined by calculating pairwise r² values between the two SNPs (e.g. located in the 17q12-21 region). The r² between two SNPs A and B is defined as r²(pₐ, p_{b}, p_{ab})=(p_{ab}−pₐp_{b})²/(pₐ(1−pₐ)p_{b}(1−p_{b}), where pₐ is the frequency of allele a of SNP A, p_{b} is the frequency of allele b of SNP B, and p_{ab} is the frequency of haplotypes (i.e. co-occurrence on one chromosome) with allele a at SNP A and b at SNP B (Hill and Robertson). An allele in linkage disequilibrium as used herein, refers to an allele that is expected to behave similarly to an effect allele and/or risk allele and is selected based on allele frequencies and/or high r² value (greater than or equal to (>) 0.6) and/or high D′ value (>0.6) with the effect and/or risk alleles and/or selected SNP as defined herein.

“Odds ratio” or “OR” when used herein refers to the ratio of the odds of the disease or manifestation of the disease, such as the presence of one or more symptoms of a disease for individuals in different groups, for example a group of individuals with the marker (allele or polymorphism) relative to the odds in individuals without the marker (allele or polymorphism), or for example in a group of individuals treated with a therapeutical product compared to a group of individuals treated with placebo.

The terms “sample” and “biological sample” are used interchangeably herein to refer to any biological sample obtained from an individual including body fluids, body tissue (e.g. hair, skin or lung samples), nasal samples (including nasal swabs or nasal polyps), sputum, cells, or other sources. Body fluids include, e.g., lymph, sera, whole fresh blood, frozen whole blood, plasma (including fresh or frozen), peripheral blood mononuclear cells, nasal fluid, saliva, bronchoalveolar lavage fluid, urine, semen, synovial fluid and spinal fluid. Methods for obtaining tissue biopsies and body fluids from mammals are well known in the art. Biological samples as used herein may be obtained before treatment with immunotherapy products, or during treatment with immunotherapy products.

A “single nucleotide polymorphism (SNP)” as used herein refers to a position in the genome where variations are found in a given population. In such a position a single nucleotide (or base) in the DNA differs between subjects. These single base changes are called SNPs or “snips.” A very large number of SNPs have been identified in the human genome. Some SNPs are found associated with various diseases or phenotypes such as eye color. Other SNPs are normal variations in the genome. A SNP involves two complementary base substitutions, one on each DNA strand. A number of databases of SNPs in the human genome exist, for example dbSNP at www.ncbi.nlm.nih.gov/SNP/and Ensembl Variation at www.ensembl.org/info/genome/variation/index.html.

A “strand” as defined herein refers to a string or chain of nucleotides, i.e. a polynucleotide composed of nucleotides, which can be of various types. A “DNA strand” as defined herein is a string of deoxyribose containing nucleotides, i.e. a polynucleotide composed of deoxyribonucleotides. When present inside a cell, genomic DNA is often found as a double helix formed by two complementary strands of deoxynucleotides held together by hydrogen bonds between G-C and A-T base pairs, however, two DNA strands may be separated, for example when during transcription or replication of DNA. An “RNA strand” is a string of ribose containing nucleotides, i.e. a polynucleotide composed of ribonucleotides.

An individual or “subject in need” as referred to herein, is an individual that may benefit from the administration of an immunotherapy product as defined herein. Such an individual may suffer from atopic disease, e.g. asthma or be at risk of suffering from an atopic disease, e.g. asthma. A subject in need may further include those subjects at risk of having an atopic disease, or those in whom the atopic disease, such as asthma is to be prevented. The subject may be any human being, male or female, infant, child, middle-aged or old. The atopic disease (such as asthma, or type of asthma) to be treated or prevented in the subject in need may relate to the age of the subject, the general health of the subject, the medications used for treating the subject and whether or not the subject, or close relatives (e.g. one or more parents), have a prior history of suffering from diseases or disorders that may have or have induced atopic disease in the individual.

The term “therapeutically effective amount” of an immunotherapy product as used herein refers to an amount sufficient to treat, cure, alleviate, prevent, reduce the risk of, or partially arrest the clinical manifestations of a given disease or disorder and its complications.

As used herein, “therapy” or “treatment” refers to clinical intervention in an attempt to alter the natural course of the individual or cell being treated, and can be performed either for prophylaxis or during the course of clinical pathology.

### Biomarkers for Use in Immunotherapy

The methods and other aspects presented herein relate to the use of biomarkers for predicting efficacy of, or selecting subjects for treatment with immunotherapy products for use in treatment of atopic diseases.

### Single Nucleotide Polymorphisms

In some examples, biomarkers for use in aspects as described herein are one or more biomarkers selected from the group consisting of single nucleotide polymorphisms (SNPs) of the 17q12-21 region, or one or more SNPs in linkage disequilibrium with a SNP of the 17q12-21 region. The 17q12-21 region is a region located on the human chromosome 17, on the “q” also called the “queue” arm, bands 12 to 21. In an more specific example, a SNP of the 17q12-21 region is located in the region between position 37830800 to position 38128700 in build GRCh37.p13 of chromosome 17. In another example a SNP of the 17q12-21 region is located in the region between position 37830800 to position 38082810 in build GRCh37.p13 of chromosome 17. When referring to ‘chromosome orientation, there are two exclusive instances: forward (or ‘Watson’) (FWD) orientation, which is the orientiation from pter (short arm telomeric end) to qter (long arm telomeric end), and the reverse (REV) orientation, which is the orientation from the qter to the pter. When a gene or a genomic DNA sequence is denoted as forward herein, it is understood that the gene orientation 5′→3′ is in the in same orientation as FWD chromosome orientation, and when a gene or genomic DNA sequence is denoted as reverse herein, it is understood that the orientation 5′→3′ is in same orientation as REV chromosome orientation. Since genomic DNA normally consists of two complementary strands or strings of nucleotides, a SNP can be detected in the forward strand and reverse strand of DNA. A skilled person will understand that the strands may be used interchangeably for detecting SNPs of the 17q12-21 region as defined herein.

Thus, one example of an aspect of the invention provides a method for selecting a subject for treatment with an immunotherapy product for treatment of an atopic disease, e.g. asthma and/or predicting response to treatment with an immunotherapy product for treatment of an atopic disease, e.g. asthma comprising:
a) Detecting whether a biological sample obtained from said subject has one or more of:
   i. A single nucleotide polymorphism (SNP) selected from the group consisting of the single nucleic acid polymorphisms located in the chromosome 17q12-21 region and/or;
   ii. A SNP in linkage disequilibrium with one or more SNPs as defined in i) above;
b) Selecting said subject for treatment with an immunotherapy product and/or predicting a beneficial response to treatment with an immunotherapy product in said subject, if one or more SNPs of step a) are detected.

The method according to the example may further comprise a treatment step c) wherein an immunotherapy product is administered to a subject that is either selected for treatment according to step b) and/or predicted to have a beneficial response to treatment according to step b).

In one example of the above method, the method is for selecting a subject for treatment with an immunotherapy product for treatment of an atopic disease, e.g. asthma.

In another of the above method, the method is for predicting response to treatment with an immunotherapy product for treatment of an atopic disease, e.g. asthma.

Another example of an aspect of the invention provides a method of treating, preventing, delaying onset, or reducing risk of an atopic disease, such as asthma, in a subject in need thereof, said method comprising the a) to d) of:
a) Obtaining and/or providing a biological sample comprising nucleic acids from said subject; and
b) Detecting whether the nucleic acids obtained from said subject has one or more of:
   i. A single nucleotide polymorphisms (SNPs) selected from the group consisting of the single nucleic acid polymorphisms located in the chromosome 17q12-21 region and/or;
   ii. A SNP in linkage disequilibrium with one or more SNPs as defined in i) above;
c) Identifying or selecting said subject for treatment with immunotherapy if one or more SNPs of section b) are identified in the nucleic acids comprised in said biological sample; and
d) Administering an immunotherapy product to said subject identified or selected according to step c).

In some examples of aspects as defined herein, one or more SNPs of the 17q12-21 region are selected from the group consisting of:
i. An allele of Table 1, i.e. rs2941504 (SEQ ID NO: 5), rs2517955 (SEQ ID NO: 4), rs2952156 (SEQ ID NO: 6), rs907092 (SEQ ID NO: 1), rs9303277 (SEQ ID NO: 13), rs12936231 (SEQ ID NO: 17), rs8069176 (SEQ ID NO: 11), rs2305480 (SEQ ID NO: 3), rs11078927 (SEQ ID NO: 14), rs11078928 (SEQ ID NO: 15), rs2290400 (SEQ ID NO: 2), rs7216389 (SEQ ID NO: 10), rs4065275 (SEQ ID NO: 9), rs8076131 (SEQ ID NO: 12), rs12603332 (SEQ ID NO: 16), rs3894194 (SEQ ID NO: 8) and rs3859192 (SEQ ID NO: 7) or a complementary SNP thereof (i.e. in the reverse strand); or
ii. A SNP in linkage disequilibrium with one or more SNPs as defined in i) above.

Table 1 below includes these SNPs as listed in i. above, and flanking regions of the SNP shown in the forward strand.

TABLE 1
A list of specific SNPs of the 17q12-21 chromosomal region

| SEQ | | Position | Upstream | | Downstream | |
|---|---|---|---|---|---|---|
| ID | | (GRCh37. | nucleotide | Allele | nucleotide | |
| NO: | SNP rsID | P13) | sequence | nucleotide | sequence | Complete sequence |
| 1 | rs907092 | 37922259 | CACCTCCC | G | GAGTCTCT | CACCTCCCCTTCCTTGTTG |
| | | | CTTCCTTG | | TGGGCAGA | ATCACTTTGACGGAGTCTC |
| | | | TTGATCAC | | TGGGCGG | TTGGGCAGATGGGCGGGG |
| | | | TTTGAC | | GGGCTTG | GCTTG |
| 2 | rs2290400 | 38066240 | ATCTCAGG | T | TCCCTCTC | ATCTCAGGGCCTTACCTCC |
| | | | GCCTTACC | | TGTGAGCC | CACTGACTCTTTTCCCTCT |
| | | | TCCCACTG | | AGCTCCTA | CTGTGAGCCAGCTCCTATT |
| | | | ACTCTT | | TTGAAG | GAAG |
| 3 | rs2305480 | 38062196 | TAAAAAGG | G | GTCCTCCA | TAAAAAGGCTGCTTAGGA |
| | | | CTGCTTAG | | TGTGTAGC | GAGGCTTGTCTGGGTCCT |
| | | | GAGAGGCT | | TCCCCGGA | CCATGTGTAGCTCCCCGG |
| | | | TGTCTG | | AATCAG | AAATCAG |
| 4 | rs2517955 | 37843681 | AACAAAAC | C | ACGTCTTC | AACAAAACGTTCCAAGATG |
| | | | GTTCCAAG | | AAAGCCGG | ATCCCTGCCCCCACGTCTT |
| | | | ATGATCCC | | AACTGACA | CAAAGCCGGAACTGACAC |
| | | | TGCCCC | | CCTTCC | CTTCC |
| 5 | rs2941504 | 37830900 | CTCTGTGA | A | ACTGTGGA | CTCTGTGAGGTCAGTGTCC |
| | | | GGTCAGTG | | CCAGAACC | CTGGTGTGGAAAACTGTG |
| | | | TCCCTGGT | | ATGCATTG | GACCAGAACCATGCATTGA |
| | | | GTGGAA | | AGGGAC | GGGAC |
| 6 | rs2952156 | 37876835 | TGTACCAG | A | TGGAGAGG | TGTACCAGACCCTGTGCTA |
| | | | ACCCTGTG | | AGAGGGTG | AGCAAGGGGGTATGGAGA |
| | | | CTAAGCAA | | ACAAGAAT | GGAGAGGGTGACAAGAAT |
| | | | GGGGGT | | ATTGGA | ATTGGA |
| 7 | rs3859192 | 38128648 | AGAGAGAT | T | GGAAGGCA | AGAGAGATATAGAAACTG |
| | | | ATAGAAAC | | CAGTCAGA | ACAGACAGAGACTGGAAG |
| | | | TGACAGAC | | AGCAGAAG | GCACAGTCAGAAGCAGAA |
| | | | AGAGAC | | TAAGAC | GTAAGAC |
| 8 | rs3894194 | 38121993 | CCCGGGCC | A | AGGGGACC | CCCGGGCCCTGGCCAGAC |
| | | | CTGGCCAG | | TGACACCA | AGCTAAACCCTCAAGGGG |
| | | | ACAGCTAA | | CTTGACAG | ACCTGACACCACTTGACAG |
| | | | ACCCTC | | CCTCAT | CCTCAT |
| 9 | rs4065275 | 38080865 | AGCCCCAG | G | ACCCCTGC | AGCCCCAGGGAGAGAATT |
| | | | GGAGAGAA | | TGGACTGT | CTCACTCAGCACGACCCCT |
| | | | TTCTCACT | | GTCTCACT | GCTGGACTGTGTCTCACTG |
| | | | CAGCAC | | GAAGGC | AAGGC |
| 10 | rs7216389 | 38069949 | AAGTGAGG | T | GCATGGAC | AAGTGAGGCAACCCTGGA |
| | | | CAACCCTG | | TCGGCCCT | AAGTCACAAACATGCATGG |
| | | | GAAAGTCA | | GATTGATC | ACTCGGCCCTGATTGATCA |
| | | | CAAACA | | AGGCAC | GGCAC |
| 11 | rs8069176 | 38080912 | GTTTATATT | G | GTTTGCTG | GTTTATATTATTATTTTTCT |
| | | | ATTATTTTT | | ATTTGTTTT | ACGGTGATTTGGTTTGCTG |
| | | | CTACGGTG | | TCTGTTTTT | ATTTGTTTTTCTGTTTTTTC |
| | | | ATTT | | TCAT | AT |
| 12 | rs8076131 | 38080912 | GTCTCACT | A | CTCTTAAG | GTCTCACTGAAGGCCTAAT |
| | | | GAAGGCCT | | CTCTTCCC | GTGGGACATCTACTCTTAA |
| | | | AATGTGGG | | ATCAGGAG | GCTCTTCCCATCAGGAGG |
| | | | ACATCT | | GGAAAG | GAAAG |
| 13 | rs9303277 | 37976469 | CTGTGTTC | C | AGGGCTGT | CTGTGTTCAGTGTCATGCG |
| | | | AGTGTCAT | | GTTAAAAT | ATGAATTAAAACAGGGCTG |
| | | | GCGATGAA | | AGTGACTG | TGTTAAAATAGTGACTGAA |
| | | | TTAAAA | | AATTGT | TTGT |
| 14 | rs11078927 | 38064405 | TAGTGTCT | C | GGGAAGCC | TAGTGTCTTTGCCCATTCT |
| | | | TTGCCCAT | | CAGCAGCT | GAAATGACGAACGGGAAG |
| | | | TCTGAAAT | | CACTCACC | CCCAGCAGCTCACTCACCT |
| | | | GACGAA | | TTCTGG | TCTGG |
| 15 | rs11078928 | 38064469 | GGATTTTG | T | AAACCAGC | GGATTTTGTTTTGCCCCTG |
| | | | TTTTGCCC | | ACCAAAAA | AAATGAATATCTAAACCAG |
| | | | CTGAAATG | | GGGAGGA | CACCAAAAAGGGAGGAAA |
| | | | AATATC | | AAAAACA | AAACA |
| 16 | rs12603332 | 38082807 | CCGAAAAC | C | GGCTGCTT | CCGAAAACTTCTGCTGCCA |
| | | | TTCTGCTG | | AGTGCTGG | TAGCTGGCAGGCGGCTGC |
| | | | CCATAGCT | | GCCCTGAT | TTAGTGCTGGGCCCTGAT |
| | | | GGCAGG | | GTGGTT | GTGGTT |
| 17 | rs12936231 | 38029120 | CTTGTAGT | C | AGTGAAAC | CTTGTAGTTACTTACATTA |
| | | | TACTTACA | | CATCAAGT | GCCCCCAGATGCAGTGAA |
| | | | TTAGCCCC | | ATAAATTAT | ACCATCAAGTATAAATTAT |
| | | | CAGATG | | TCAAA | TCAAA |

In some examples of the aspects as defined herein, the SNPs in linkage disequilibrium with one or more SNPs of Table 1, is located in the chromosome 17q12-21 region.

In another specific example, when used in aspects of as defined herein, a SNP of the 17q12-21 region is one or more of the SNPs of the 17q12-21 region of Table 1 and selected from the group consisting of: rs2941504 (SEQ ID NO: 5), rs2517955 (SEQ ID NO: 4), rs2952156 (SEQ ID NO: 6), rs907092 (SEQ ID NO: 1), rs9303277 (SEQ ID NO: 13), rs12936231 (SEQ ID NO: 17), rs8069176 (SEQ ID NO: 11), rs2305480 (SEQ ID NO: 3), rs11078927 (SEQ ID NO: 14), rs11078928 (SEQ ID NO: 15), rs2290400 (SEQ ID NO: 2), rs7216389 (SEQ ID NO: 10), rs4065275 (SEQ ID NO: 9), rs8076131 (SEQ ID NO: 12), rs12603332 (SEQ ID NO: 16), rs3894194 (SEQ ID NO: 8) and rs3859192 (SEQ ID NO: 7), or a complementary SNP thereof, and SNPs in linkage disequilibrium with one or more SNPs above.

In another specific example, when used in aspects of as defined herein, a SNP of the 17q12-21 region is one or more SNPs of the 17q12-21 region of Table 1 and selected from the group consisting of: rs2941504 (SEQ ID NO: 5), rs2517955 (SEQ ID NO: 4), rs2952156 (SEQ ID NO: 6), rs907092 (SEQ ID NO: 1), rs9303277 (SEQ ID NO: 13), rs12936231 (SEQ ID NO: 17), rs8069176 (SEQ ID NO: 11), rs2305480 (SEQ ID NO: 3), rs11078927 (SEQ ID NO: 14), rs11078928 (SEQ ID NO: 15), rs2290400 (SEQ ID NO: 2), rs7216389 (SEQ ID NO: 10), rs4065275 (SEQ ID NO: 9), rs8076131 (SEQ ID NO: 12), rs12603332 (SEQ ID NO: 16), rs3894194 (SEQ ID NO: 8) and rs3859192 (SEQ ID NO: 7), as shown in of Table 1, or a complementary SNP thereof.

In another specific example, when used in aspects as defined herein, a SNP of the 17q12-21 region is one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2), rs7216389 (SEQ ID NO: 10), rs3894194 (SEQ ID NO: 8) and rs3859192 (SEQ ID NO: 7), or a complementary SNP thereof and SNPs in linkage disequilibrium with one or more SNPs thereof.

In another example, when used in aspects as defined herein, a SNP of the 17q12-21 region is one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2), rs7216389 (SEQ ID NO: 10), rs3894194 (SEQ ID NO: 8) and rs3859192 (SEQ ID NO: 7) or a complementary SNP thereof.

In other examples, when used in aspects as defined herein, a SNP of the 17q12-21 region is one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10), or a complementary SNP in the reverse strand and SNPs in linkage disequilibrium with one or more SNPs thereof.

In other examples, when used in aspects as defined herein, a SNP of the 17q12-21 region is one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10) or a complementary SNP in the reverse strand.

In other examples, when used in aspects as defined herein, a SNP of the 17q12-21 region is rs7216389 (SEQ ID NO: 10) and SNPs in linkage disequilibrium with rs7216389 (SEQ ID NO: 10) or a complementary SNP in the reverse strand.

In another example of aspects as defined herein, a SNP of the 17q12-21 region is at least the rs7216389 (SEQ ID NO: 10) T effect allele or a complementary SNP in the reverse strand.

In one specific example, when one or more SNPs of the 17q12-21 region is used in aspects as defined herein, detection of a SNP involves the detection or identification of a heterozygote or a homozygote with respect to one or more of the SNPs of the 17q12-21 region, such as a heterozygote or a homozygote with respect to one or more of the effect allele of the SNPs of Table 1. For example, a specific subject could be a heterozygote with respect to the rs7216389 (SEQ ID NO: 10) SNP of Table 1, i.e. one version of chromosome 17 in the subject has the rs7216389 (SEQ ID NO: 10) effect allele of Table 1, and the other version of chromosome 17 in the subject does not have the rs7216389 (SEQ ID NO: 10) effect allele.

In one example when one or more SNPs of the 17q12-21 region is used in aspects as defined herein, the detection of one or more SNPs of the 17q12-21 region involves the detection or identification of a subject being a homozygote with respect to one or more of the SNPs of the 17q12-21 region, e.g. a subject being a homozygote with respect to one or more of the SNPs (or effect alleles) of Table 1 above. For example, the subject could be a rs7216389 (SEQ ID NO: 10) SNP homozygote, i.e. both versions of chromosome 17 in the subject have the rs7216389 (SEQ ID NO: 10) SNP T effect allele associated with increased effect of immunotherapy treatment.

In another example when one or more SNPs of the 17q12-21 region is used in aspects as defined herein, the detection of one or more SNPs of the 17q12-21 region involves the detection or identification of a subject being a homozygote with respect to one or more of the SNPs in Table 1 and selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10), and SNPs in linkage disequilibrium with one or more SNPs thereof.

In another example when one or more SNPs of the 17q12-21 region is used in aspects as defined herein, a subject is selected for treatment and/or a beneficial response is predicted in the subject if is detected that the subject is a homozygote with respect to one or more of the SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10) or a SNP in linkage disequilibrium with one or more of the SNPs.

In another example when one or more SNPs of the 17q12-21 region is used in aspects as defined herein, a subject is selected for treatment and/or a beneficial response is predicted in the subject if is detected that the subject is a homozygote with respect to one or more of the SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10).

In another example when one or more SNPs of the 17q12-21 region is used in aspects as defined herein, the detection of one or more SNPs of the 17q12-21 region involves the detection or identification of a subject being a homozygote with respect to one or more of the SNPs in Table 1 and selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10). Thus, the detection of one or more SNPs of the 17q12-21 region involves the detection of a subject being a rs907092 (SEQ ID NO: 1) homozygote, or a subject being a rs9303277 (SEQ ID NO: 13) homozygote, or a subject being a rs9303277 (SEQ ID NO: 13) homozygote, or a subject being a rs8069176 (SEQ ID NO: 11) homozygote, or a subject being a rs2305480 (SEQ ID NO: 3) homozygote, or a subject being a rs11078927 (SEQ ID NO: 14) homozygote, or a subject being a rs2290400 (SEQ ID NO: 2) homozygote or a subject being a rs7216389 (SEQ ID NO: 10) homozygote.

In some examples of the aspects as defined herein, detection of one or more SNPs of the 17q12-21 region involves the detection, or identification, of a subject being a homozygote with respect to at least the rs7216389 (SEQ ID NO: 10) effect allele.

In another example of the aspects as defined herein, a subject is selected from treatment and/or a beneficial response is predicted in the subject if is detected that the subejct is a homozygote with respect to the rs7216389 (SEQ ID NO: 10) effect allele or a SNP in linkage disequilibrium with the rs7216389 (SEQ ID NO: 10) effect allele.

In another example of the aspects as defined herein, a subject is selected from treatment and/or a beneficial response is predicted in the subject if is detected that the subejct is a homozygote with respect to the rs7216389 (SEQ ID NO: 10) effect allele.

In one example, when one or more SNPs of the 17q12-21 region is used in aspects as defined herein, the subject is having 1 or more, such as 2, 3, 4, 5, 6, 7, 8, 9 or more of the SNPs selected from the Table 1 and SNPs in linkage disequilibrium with one or more of the SNPs of Table 1.

In one example, when one or more SNPs of the 17q12-21 region is used in aspects as defined herein, the subject is a having 1 or more, such as 2, 3, 4, 5, 6, 7, 8, 9 or more of the SNPs selected from the group consisting of: rs2941504 (SEQ ID NO: 5), rs2517955 (SEQ ID NO: 4), rs2952156 (SEQ ID NO: 6), rs907092 (SEQ ID NO: 1), rs9303277 (SEQ ID NO: 13), rs12936231 (SEQ ID NO: 17), rs8069176 (SEQ ID NO: 11), rs2305480 (SEQ ID NO: 3), rs11078927 (SEQ ID NO: 14), rs11078928 (SEQ ID NO: 15), rs2290400 (SEQ ID NO: 2), rs7216389 (SEQ ID NO: 10), rs4065275 (SEQ ID NO: 9), rs8076131 (SEQ ID NO: 12), rs12603332 (SEQ ID NO: 16), rs3894194 (SEQ ID NO: 8) and rs3859192 (SEQ ID NO: 7), and SNPs in linkage disequilibrium with one or more SNPs above.

In another example, when one or more SNPs of the 17q12-21 region is used in aspects as defined herein, the subject is a having 1 or more, such as 2, 3, 4, 5, 6, 7, 8, 9 or more of the SNPs selected from the group consisting of: rs2941504 (SEQ ID NO: 5), rs2517955 (SEQ ID NO: 4), rs2952156 (SEQ ID NO: 6), rs907092 (SEQ ID NO: 1), rs9303277 (SEQ ID NO: 13), rs12936231 (SEQ ID NO: 17), rs8069176 (SEQ ID NO: 11), rs2305480 (SEQ ID NO: 3), rs11078927 (SEQ ID NO: 14), rs11078928 (SEQ ID NO: 15), rs2290400 (SEQ ID NO: 2), rs7216389 (SEQ ID NO: 10), rs4065275 (SEQ ID NO: 9), rs8076131 (SEQ ID NO: 12), rs12603332 (SEQ ID NO: 16), rs3894194 (SEQ ID NO: 8) and rs3859192 (SEQ ID NO: 7).

In another example, when one or more SNPs of the 17q12-21 region is used in aspects as defined herein, the subject is a having 1 or more, such as 2, 3, 4, 5 or 6, of the SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10).

In some examples, SNPs in linkage disequilibrium with a SNP of the 17q12-21 region may be used in aspects as defined herein. As mentioned above, a SNP or allele in linkage disequilibrium as used herein, refers to an allele that is expected to behave similarly to a risk and/or effect allele and is selected based on allele frequencies and/or high r² value (greater than or equal to (>) 0.6) and/or high D′ value (>0.6) with the risk alleles, effect alleles and/or selected SNP as defined herein. In one embodiment, the high r² value is between about 0.6 to about 1.0, for example such as larger than about 0.6, or larger than about 0.7, or between about 0.8 to 1, such as larger than about 0.8, larger than about 0.9, or between about 0.9 and about 1. In one embodiment, the high D′ value is is between about 0.6 to about 1.0, for example such as larger than about 0.6, or larger than about 0.7, or between about 0.8 to about 1, such as larger than about 0.8, or larger than about 0.9, or between about 0.9 and about 1, such as about 0.9, about 0.93, about 0.95, about 0.98 and about 1.

SNP or allele in linkage disequilibrium as used in aspects defined herein, may be a SNP in linkage disequilibrium with one or more SNPs as shown in Table 1 selected from the group consisting of rs2941504 (SEQ ID NO: 5), rs2517955 (SEQ ID NO: 4), rs2952156 (SEQ ID NO: 6), rs907092 (SEQ ID NO: 1), rs9303277 (SEQ ID NO: 13), rs12936231 (SEQ ID NO: 17), rs8069176 (SEQ ID NO: 11), rs2305480 (SEQ ID NO: 3), rs11078927 (SEQ ID NO: 14), rs11078928 (SEQ ID NO: 15), rs2290400 (SEQ ID NO: 2), rs7216389 (SEQ ID NO: 10), rs4065275 (SEQ ID NO: 9), rs8076131 (SEQ ID NO: 12), rs12603332 (SEQ ID NO: 16), rs3894194 (SEQ ID NO: 8) and rs3859192 (SEQ ID NO: 7). In other examples, a SNP or allele in linkage disequilibrium as used in aspects defined herein, may be a SNP in linkage disequilibrium with one or more SNPs selected from the group consisting of the SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10).

In other more specific examples, a SNP or allele in linkage disequilibrium as used in aspects defined herein, may be a SNP in linkage disequilibrium with rs2941504 (SEQ ID NO: 5), or a SNP in linkage disequilibrium with rs2517955 (SEQ ID NO: 4), or a SNP in linkage disequilibrium with rs2952156 (SEQ ID NO: 6), or a SNP in linkage disequilibrium with rs907092 (SEQ ID NO: 1), or a SNP in linkage disequilibrium with rs9303277 (SEQ ID NO: 13), or a SNP in linkage disequilibrium with rs12936231 (SEQ ID NO: 17), or a SNP in linkage disequilibrium with rs8069176 (SEQ ID NO: 11), or a SNP in linkage disequilibrium with rs2305480 (SEQ ID NO: 3), or a SNP in linkage disequilibrium with rs11078927 (SEQ ID NO: 14), or a SNP in linkage disequilibrium with rs11078928 (SEQ ID NO: 15), or a SNP in linkage disequilibrium with rs2290400 (SEQ ID NO: 2), or a SNP in linkage disequilibrium with rs7216389 (SEQ ID NO: 10), or a SNP in linkage disequilibrium with rs4065275 (SEQ ID NO: 9), or a SNP in linkage disequilibrium with rs8076131 (SEQ ID NO: 12), or a SNP in linkage disequilibrium with rs12603332 (SEQ ID NO: 16), or a SNP in linkage disequilibrium with rs3894194 (SEQ ID NO: 8), or a SNP in linkage disequilibrium with rs3859192 (SEQ ID NO: 7).

SNPs detected in the aspects described herein can be characterized by the use of any suitable method capable of detecting SNPs. Such methods include the direct or indirect sequencing of the site; hybridization-based methods, such as dynamix allele-specific hybridization, molecular beacons, and SNP microarrays; enzyme-based methods such as restriction fragment length polymorphism, polymerase chain reaction (PCR)-based methods such as flap endonuclease, primer extension, 5′-nuclease and oligonucleotide ligation assay; other post-amplification methods based on the physical properties of DNA, such as single strand conformation polymorphism, temperature gradient gel electrophoresis, denaturing high performance liquid chromatography, high-resolution melting of the entire amplicon, use of DNA mis-match proteins (e.g. antibodies), SNPlex and surveyor nuclease assay.

Many of these above mentioned methods include the use of strings of nucleic acids or proteins capable of binding to or interacting with a specific sequence of nucleotides in a DNA or RNA strand. Therefore, in a specific example of the aspects as defined herein, detection of one or more SNPs of the 17q12-21 region involves the use of one or more strings of nucleotides (e.g. DNA or RNA) capable of binding to or interacting with sequences of Table 1 above, or fragments thereof, and/or complementary sequences thereof.

One particular example of a detection method involves the use of the Taq DNA polymerase's 5′-nuclease activity, which is for example used in the TaqMan assay for SNP genotyping. TaqMan assays are commercially available for a high number of SNPs, including SNPs of the 17q12-21 region. The TaqMan assay is performed concurrently with a PCR reaction and the results can be read in real-time as the PCR reaction proceeds (McGuigan & Ralston 2002). The assay requires forward and reverse PCR primers that will amplify a region that includes the SNP polymorphic site. Allele discrimination is achieved using fluorescence resonance energy transfer (FRET) combined with one or two allele-specific probes that hybridize to the SNP polymorphic site. The probes will have a fluorophore linked to their 5′ end and a quencher molecule linked to their 3′ end. While the probe is intact, the quencher will remain in close proximity to the fluorophore, eliminating the fluorophore's signal. During the PCR amplification step, if the allele-specific probe is perfectly complementary to the SNP allele, it will bind to the target DNA strand and then get degraded by 5′-nuclease activity of the Taq polymerase as it extends the DNA from the PCR primers. The degradation of the probe results in the separation of the fluorophore from the quencher molecule, generating a detectable signal. If the allele-specific probe is not perfectly complementary, it will have lower melting temperature and not bind as efficiently. This prevents the nuclease from acting on the probe (McGuigan & Ralston 2002). Since the TaqMan assay is based on PCR, it is relatively simple to implement. The TaqMan assay can be multiplexed by combining the detection of up to seven SNPs in one reaction. The scale of the assay can be drastically increased by performing many simultaneous reactions in microtitre plates.

Detection and quantifying SNP markers in biological samples may be performed at different time points before or during the progression of disease, or at different stages before, during or after treatment. In some examples of the aspects as defined herein, the biological samples are obtained prior to treatment with an immunotherapy product as defined herein. In some examples of the aspects as defined herein, detection and quantifying SNP markers is performed prior to treatment with an immunotherapy product as defined herein.

### Th2 Inflammation Biomarkers

Type 2 T helper cells (Th2) cells are capable of orchestrating protective so-called “type 2” immune responses, such as for example those that target helmiths and facilitate tissue repair, but also contribute to chronic inflammatory diseases such as asthma and allergy. Biomarkers associated with a Th2 response are for example the level or concentration of blood eosinophils, eosinophil cationic protein (ECP), tryptase, periostin, and total IgE, which are all related to a Th2 response.

One example of an aspect according to the invention provides a method for selecting a subject for treatment with an immunotherapy product for treatment of an atopic disease, e.g. asthma and/or predicting response to treatment with an immunotherapy product for treatment of an atopic disease, e.g. asthma comprising:
a) Quantifying the level of one or more Th2 markers selected from the group consisting of eosinophil count, eosinophil cationic protein (ECP), tryptase, periostin, and total IgE;
b) Selecting said subject for treatment with an immunotherapy product and/or predicting a beneficial response to treatment with an immunotherapy product in said subject, if the level of one or more markers of step a) is increased, equal to, or above, a reference level.

In one example of the above method, the method is for selecting a subject for treatment with an immunotherapy product for treatment of an atopic disease, e.g. asthma.

In another of the above method, the method is for predicting response to treatment with an immunotherapy product for treatment of an atopic disease, e.g. asthma.

In some examples, the method above further comprises a treatment step c) wherein an immunotherapy product is administered to a subject that is either selected for treatment according to step b) and/or predicted to have a beneficial response to treatment according to step b).

Another example of an aspect according to the prevention, provides a method of treating, preventing, delaying onset, and/or reducing risk of an atopic disease, such as asthma, in a subject in need thereof, said method comprising:
a) Obtaining and/or providing a biological sample from said subject; and
b) Quantifying the level of one or more Th2 markers selected from the group consisting of eosinophils, eosinophil cationic protein (ECP), tryptase, periostin, and total IgE;
c) Identifying or selecting said subject for treatment with an immunotherapy product and/or predicting a beneficial response to treatment with an immunotherapy product in said subject, if the level of one or more markers of step a) is increased, equal to, or above, a reference level; and
d) Administering an immunotherapy product to said subject identified or selected according to step c).

In one example of the method above, the method is for treating, preventing, delaying onset, and/or reducing risk of asthma, e.g. allergic asthma.

Detection and quantifying Th2 markers (i.e blood eosinophils, eosinophil cationic protein (ECP), tryptase, periostin, and IgE) in biological samples may be performed at different time points during the progression of disease, or at different stages of treatment. In some examples of the aspects as defined herein, the biological samples are obtained prior to treatment with an immunotherapy product as defined herein.

Detection and quantifying Th2 markers (i.e blood eosinophils, eosinophil cationic protein (ECP), tryptase, periostin, and IgE) in biological samples may be performed using any suitable standard methods in the art. For example, periostin, ECP, tryptase and IgE (such as total IgE) may be measured using immunoassays, such as sandwich immunoassays (e.g. ImmunoCap, Thermo Scientific). Similarly, periostin may be measured by using ELISA (e.g. using Human Periostin/OSF-2 DuoSet ELISA, R&D Systems, DY3548B). Blood eosinophils may be counted by using standard methods, such as for example manually counting of eosinophils in a blood smear, by automated laboratory methods, e.g. by flow cytometric methods, such as automated flouresence flow cytometric methods, by using a near-patient device, e.g. NPT HemoCue® WBC Diff device (HemoCue AB, Angelholm, Sweden), or other standard methods used in the art.

In some examples of aspects as defined herein, a method involves a step comprising detection of, quantifying, counting, or measuring the concentration of one or more Th2 markers selected from the group consisting of eosinophils, eosinophil cationic protein (ECP), tryptase, periostin, and total IgE in a sample.

In some examples of aspects as defined herein, a method involves a step comprising detection of, quantifying, counting, or measuring the concentration of one or more Th2 markers selected from the group consisting of eosinophils, eosinophil cationic protein (ECP), tryptase, and periostin in a sample.

In some examples of the aspects as defined herein, such a method involves a step comprising detection, quantifying, or counting number of eosinophils. In some examples of the aspects as defined herein, such a method involves a step comprising detection of, quantifying, or measuring the concentration of eosinophil cationic protein (ECP). In some examples of the aspects as defined herein, such a method involves a step comprising detection of, quantifying, or measuring the concentration of tryptase. In some examples of the aspects as defined herein, such a method involves a step comprising detection of, quantifying, or measuring the concentration of periostin. In some examples of the aspects as defined herein, such a method involves a step comprising detection of, quantifying, or measuring the concentration of IgE, preferably total IgE.

Measurement or detection of Th2 markers when used in aspects as defined herein, may be performed in any suitable biological sample. In preferred examples of the aspects as defined herein, one or more Th2 markers are detected or measured in whole blood, blood serum or blood plasma. In preferred examples of the aspects as defined herein, detection of, counting, or measuring the level of eosinophils is done in sputum or whole blood, preferably whole blood, or whole blood treated with anti-coagulant, e.g. EDTA. In preferred examples of the aspects as defined herein, ECP, tryptase, periostin, and/or IgE is determined in blood serum.

In certain examples of aspects relating to Th2 markers as defined herein, the term “reference level” herein refers to a predetermined value. As the skilled artisan will appreciate, the reference level is predetermined and set to meet the requirements in terms of, for example, specificity and/or sensitivity. These requirements can vary, e.g., from regulatory body to regulatory body. It may be, for example, that assay sensitivity or specificity, respectively, has to be set to certain limits, e.g., 80%, 90% or 95%. These requirements may also be defined in terms of positive or negative predictive values. Nonetheless, based on the teaching given in the present invention it will always be possible to arrive at the reference level meeting those requirements. In some examples of aspects as defined herein, the reference level is determined in a group of healthy subjects. In some examples of aspects as defined herein, the reference level is predetermined in a group of randomly selected subjects. The reference value in one embodiment has been predetermined in the disease entity to which the patient belongs (e.g., an atopic disease, such as selected from the group consisting of allergic rhinitis, allergic conjunctivitis and allergic asthma, or more specifically allergic asthma, or more specifically moderate or severe allergic asthma). In some examples of aspects as defined herein, the reference level is predetermined in a group of subjects diagnosed with the same atopic disease as said subject. In some examples of aspects as defined herein, the reference level is predetermined in a group of subjects diagnosed with one or more atopic diseases. In some examples of aspects as defined herein, the reference level is predetermined in a group of subjects diagnosed with one or more diseases selected from the group consisting of allergic rhinitis, allergic conjunctivitis and allergic asthma. In some examples of aspects as defined herein, the reference level is predetermined in a group of subjects diagnosed with allergic asthma. In some examples of aspects as defined herein, the reference level is predetermined in a group of subjects diagnosed with moderate or severe allergic asthma.

In certain embodiments, the reference level can be set to any percentage between, e.g., 20%, 25% and 75% of the overall distribution of the values in a disease entity investigated. In other embodiments, the reference level can be set to, for example, the median, tertiles, quartiles, or quintiles as determined from the overall distribution of the values in a disease entity investigated or in a given population. In one embodiment, the reference level is set to the median value as determined from the overall distribution of the values in a disease entity investigated. In one embodiments, the reference level may depend on the gender or age of the patient, e.g., males may have a different reference level than females. In some examples of the aspects as defined herein the reference level is the mean or median value in a group of subjects.

In certain examples of aspects relating to Th2 markers as defined herein, the term “increase” or “above” refers to a level at the reference level or to an overall increase of 5%, 10%, 20%, 25%, 30%, 40%, 50%, 60%, 70%, 80%, 85%, 90%, 95%, 100% or greater, in the level of a marker (e.g., eosinophil count, ECP, tryptase, periostin, total IgE) detected by the methods described herein, as compared to the level from a reference sample.

In certain examples of the aspects relating to Th2 markers as defined herein, the term “decrease” or “below” herein refers to a level below the reference level or to an overall reduction of 5%, 10%, 20%, 25%, 30%, 40%, 50%, 60%, 70%, 80%, 85%, 90%, 95%, 96%, 97%, 98%, 99% or greater, in the level of a marker (e.g., e.g., eosinophil count, ECP, tryptase, periostin, total IgE) detected by the methods described herein, as compared to the level from a reference sample.

The level of eosinophils counted in a biological sample, such as whole blood may vary depending on the degree of Th2 mediated inflammation in a subject. In some examples of aspects as defined herein, when the level of eosinophil count is used in methods as defined herein, the reference level for eosinophil count, e.g. in whole blood, is 150 cells/microliter or higher, such as above 200 cells/microliter, such as from 200 cells/microliter to 500 cells/microliter, such as 225 cells/microliter, such as 250 cells/microliter, such as 275 cells/microliter, or such as from 200 to 300 cells/microliter, such as 225 cells/microliter, such as 250 cells/microliter, or such as 275 cells/microliter, or such as from 300 to 400 cells/microliter, such as 325 cells/microliter, such as 350 cells/microliter, or such as 375 cells/microliter, such as from 400 to 500 cells/microliter, such as 425 cells/microliter, such as 450 cells/microliter, or such as 475 cells/microliter, or such as 600 to 800 cells/microliter or more.

In some examples, when the level of eosinophil count is used in methods as defined herein, the reference level for eosinophil count, e.g. in whole blood, is from 100 cells/microliter to 300 cells/microliter, such as about 150 cells/microliter, or such as about 200 cells/microliter.

In some examples, when the level of eosinophil count is used in methods as defined herein, the reference level for eosinophil count, e.g. in whole blood, is from 300 cells/microliter to 500 cells/cells/microliter, such as about 325 cells/microliter, such as about 350 cells/microliter, or such as about 375 cells/microliter, such as about 400 cells/microliter, such, such as about 425 cells/microliter, such as about 450 cells/microliter, or such as about 475 cells/microliter.

The level of eosinophil cationic protein (ECP) in a biological sample, e.g. a blood serum sample may vary depending on the degree of Th2 mediated inflammation in a subject. In some examples, when the level of eosinophil cationic protein (ECP) is used in methods as defined herein, the reference level for eosinophil cationic protein (ECP), e.g. in blood serum, is 3 micrograms/L or higher, such as from 3 to 8 micrograms/L, such as 4 micrograms/L, 5 micrograms/L, 6 micrograms/L or 7 micrograms/L, or such as from 8 to 14 micrograms/L, such as 8 micrograms/L, 9 micrograms/L, 10 micrograms/L, 11 micrograms/L, 12 micrograms/L, 13 micrograms/L, or from 14 micrograms/L or higher, such as from 14 to 20 micrograms/L, such as 14 to 16 micrograms/L, such as 15 micrograms/L, or such as from 16 to 18 micrograms/L, such as 17 micrograms/L, or such as from 18 to 20 micrograms/L, such as 19 micrograms/L, or such as from 20 to 40 micrograms/L, such as from 20 to 30 micrograms/L, such as 20 to 22 micrograms/L, such as 21 micrograms/L, or such as from 22 to 24 micrograms/L, such as 23 micrograms/L, or such as from 24 to 26 micrograms/L, such as 25 micrograms/L, or such as from 26 to 28 micrograms/L, such as 27 micrograms/L, or such as from 28 to 30 micrograms/L, e.g. 29 micrograms/L, or such as from 30 to 40 micrograms/L, such as 30 to 32 micrograms/L, such as 33 micrograms/L, or such as from 32 to 34 micrograms/L, such as 33 micrograms/L, or such as from 34 to 36 micrograms/L, such as 35 micrograms/L, or such as from 36 to 38 micrograms/L, such as 37 micrograms/L, or such as from 38 to 40 micrograms/L, such as 39 micrograms/L, or such as above 40 micrograms/L, such as above 50 micrograms/L.

In some examples, when the level of eosinophil cationic protein (ECP) is used in methods as defined herein, the reference level for eosinophil cationic protein (ECP), e.g. in blood serum, is from 10 to 15 micrograms/L, such as about 14 micrograms/L, such as 14. 4 micrograms/L.

In some examples, when the level of eosinophil cationic protein (ECP) is used in methods as defined herein, the reference level for eosinophil cationic protein (ECP), e.g. in blood serum, is from 25 micrograms/L to about 35 micrograms/L, such as about 29 micrograms/L, such as 29. 4 micrograms/L.

The level of periostin in a biological sample, e.g. a blood serum sample may vary depending on the degree of Th2 mediated inflammation in a subject. In some examples, when the level of periostin is used in methods as defined herein, the reference level for periostin, e.g. in blood serum, is above 15 nanograms/mL, such as from 15 to 40 nanograms/mL, such as from 15 to 17 nanograms/mL, or such as from 17 to 20 nanograms/mL, or such as from 20 to 35 nanograms/mL, such as 20 nanograms/mL, or such as 21 nanograms/mL, such as 22 nanograms/mL, such as 23 nanograms/mL, such as 24 nanograms/mL, such as 25 nanograms/mL, such as 26 nanograms/mL, such as 27 nanograms/mL, such as 28 nanograms/mL, such as 29 nanograms/mL, such as 30 nanograms/mL, such as 31 nanograms/mL, such as 32 nanograms/mL, such as 33 nanograms/mL, such as 34 nanograms/mL, or such as from 35 to 40 nanograms/mL, or such as above 40 nanograms/mL.

In some examples, when the level of periostin is used in methods as defined herein, the reference level for perisotin, e.g. in blood serum, is from 15 to 25 nanograms/mL, such as about 21 nanograms/mL.

In some examples, when the level of periostin is used in methods as defined herein, the reference level for perisotin, e.g. in blood serum, is from 22 nanograms/mL to 32 nanograms/mL, such as about 27 nanograms/mL.

The level of tryptase in a biological sample, e.g. a blood serum sample may vary depending on the degree of Th2 mediated inflammation in a subject. In some examples, when the level of tryptase is used in methods as defined herein, the reference level for tryptase, e.g. in blood serum, is above 1 micrograms/L, such as 1 micrograms/L to 4 micrograms/L, such as 1 micrograms/L to 2 micrograms/L, such as 1 micrograms/L to 1.5 micrograms/L, such as 1.1 micrograms/L, 1.2 micrograms/L, 1.3 micrograms/L, or 1.4 micrograms/L, or such as 1.5 micrograms/L to 2 micrograms/L, such as 1.5 micrograms/L, 1.6 micrograms/L, 1.7 micrograms/L, 1.8 micrograms/L, or 1.9 micrograms/L, or such as 2 micrograms/L to 4 micrograms/L, such as 2 micrograms/L to 3.5 micrograms/L, such as 2.1 micrograms/L to 2.5 micrograms/L, such as 2.2 micrograms/L, such as 2.3 micrograms/L, or such as 2.4 micrograms/L, or such as 2.5 micrograms/L to 3.5 micrograms/L, such as 2.6 micrograms/L, 2.47 micrograms/L, 2.48 micrograms/L, 2.49 micrograms/L, or such as 3 micrograms/L to 3.5 micrograms/L, such as 3.1 micrograms/L, 3.2 micrograms/L, 3.3 micrograms/L, 3.4 micrograms/L, or such as from 3.5 micrograms/L to 4 micrograms/L, such as such as 3.6 micrograms/L, 3.7 micrograms/L, 3.8 micrograms/L, 3.9 micrograms/L, or such as above 4 micrograms/L, such as from 4 micrograms/L to 8 micrograms/L, such as 5 micrograms/L, 6 micrograms/L, 7 micrograms/L, or above 8 micrograms/L such as 9 micrograms/L, or above.

In some examples, when the level of tryptase is used in methods as defined herein, the reference level for tryptase, e.g. in blood serum, is from 1 micrograms/L to 4 micrograms/L, such as about 2 micrograms/L, or such as about 3 micrograms/L, or such as about 3.5 micrograms/L.

The level of total IgE in a biological sample, e.g. a blood serum sample may vary depending on the degree of Th2 mediated inflammation in a subject, or exposure to one or more allergens. In some examples, when the level of total IgE is used in methods as defined herein, the reference level for total IgE, e.g. in blood serum, is from 0.35 kU/L or higher, such as from 0.35 kU/L to 100 kU/L, such as above 1 kU/L, such as 25 kU/L, such as 50 kU/L, or such as 75 kU/L, or such as from 100 kU/L to 150 kU/L, such as 125 kU/L, or such as from 150 kU/L to 600 kU/L, such as from 150 kU/L to 250 kU/L, such as 175 kU/L, or such as 200 kU/L, or such as 225 kU/L, or such as from 250 kU/L to 600 kU/L, such as 275 kU/L, or such as 300 kU/L, or such as 325 kU/L, or such as from 350 kU/L to 600 kU/L, such as 375 kU/L, or such as 400 kU/L, or such as from 400 kU/L to 600 kU/L, such as 425 kU/L, such as 450 kU/L, such as 450 kU/L, such as 460 kU/L, such as 461 kU/L, such as 462 kU/L, such as 463 kU/L, such as 464 kU/L, such as 465 kU/L, such as 466 kU/L, such as 467 kU/L, such as 468 kU/L, such as 469 kU/L, such as 470 kU/L, such as 475 kU/L, such as 480 kU/L, such as 490 kU/L, or such as from 500 kU/L to 600 kU/L, such as 525 kU/L, such as 550 kU/L, or such as 575 kU/L, or above 600 kU/L.

In some examples, when the level of total IgE is used in methods as defined herein, the reference level for total IgE, e.g. in blood serum, is from about 0.35 kU/L to about 10 kU/L, such as above 1 kU/L.

In some examples, when the level of total IgE is used in methods as defined herein, the reference level for total IgE, e.g. in blood serum, is from about 150 kU/L to about 200 kU/L, such as about 186 kU/L.

In some examples, when the level of total IgE is used in methods as defined herein, the reference level for total IgE, e.g. in blood serum, is from about 400 kU/L to about 500 kU/L, is about 464 kU/L.

The Th2 markers when used in aspects of the invention may correlate with a beneficial effect of treatment with immunotherapy products as defined herein. In some examples of the aspects as defined herein, a method or use involves as step of detecting, quantifying the level or measuring the concentration of one or more Th2 markers selected from the group consisting of eosinophil count, eosinophil cationic protein (ECP), tryptase, and periostin. In some examples of aspects as defined herein, a method or use involves as step of detecting, quantifying the level of, or counting at least the level of eosinophils. In some examples of aspects as defined herein, a method or use involves as step of detecting, quantifying the level or measuring the concentration of at least the level of eosinophil cationic protein (ECP), e.g. serum eosinophil cationic protein (ECP). In some examples of aspects as defined herein, a method or use involves as step of detecting, quantifying the level or measuring the concentration of at least the level of tryptase, e.g. serum tryptase. In some examples of aspects as defined herein, a method or use involves as step of detecting, quantifying the level or measuring the concentration of at least the level of periostin, e.g. serum periostin. In some examples of aspects as defined herein, a method or use involves as step of detecting, quantifying the level or measuring the concentration of at least the level of total IgE, e.g. total IgE in serum.

The quantification and comparison of the quantified levels of at least two Th2 biomarkers with reference levels in aspects as defined herein may result in improved identification or selection of patients who are likely to experience a beneficial effect of treatment with immunotherapy products as described herein. In some examples of aspects as defined herein, a method or use involves as step of detecting, counting, quantifying the level or measuring the concentration of two or more Th2 biomarkers selected from the group consisting of eosinophils, eosinophil cationic protein (ECP), tryptase and periostin. Thus, in some examples of aspects as defined herein, a method or use involves as step of counting eosinophils and quantifying the level of eosinophil cationic protein (ECP). In some examples of aspects as defined herein, a method or use involves as step of counting eosinophils and quantifying the level of tryptase. In some examples of aspects as defined herein, a method or use involves as step of counting eosinophils and quantifying the level of periostin. In some examples of aspects as defined herein, a method or use involves as step of detecting, quantifying the level of, or measuring the concentration of eosinophil cationic protein (ECP) and tryptase. In some examples of aspects as defined herein, a method or use involves as step of detecting, quantifying the level of, or measuring the concentration of eosinophil cationic protein (ECP) and periostin. In some examples of aspects as defined herein, a method or use involves as step of detecting, quantifying the level of, or measuring the concentration of tryptase and periostin.

The combination of even more Th2 markers may result in improved identification or selection of a subject having an likelihood of beneficial effect of treatment with immunotherapy product. In some examples of aspects as defined herein, a method or use involves as step of detecting, counting, quantifying the level of, or measuring the concentration of three or more Th2 markers selected from the group consisting of eosinophil count, eosinophil cationic protein (ECP), tryptase and periostin. Thus in some examples of aspects as defined herein, a method or use involves as step of detecting, counting, quantifying the level of, or measuring the concentration of eosinophil count, eosinophil cationic protein (ECP) and tryptase. In some examples of aspects as defined herein, a method or use involves as step of detecting, counting, quantifying the level of, or measuring the concentration of eosinophil count, eosinophil cationic protein (ECP) and periostin. In some examples of aspects as defined herein, a method or use involves as step of detecting, counting, quantifying the level of, or measuring the concentration of eosinophil count, tryptase, and periostin. In some examples of aspects as defined herein, a method or use involves as step of detecting, counting, quantifying the level of, or measuring the concentration of eosinophil cationic protein (ECP), tryptase and periostin. In some examples of aspects as defined herein, a method or use involves as step of detecting, counting, quantifying the level of, or measuring the concentration of eosinophils, eosinophil cationic protein (ECP), tryptase and periostin.

In some examples of aspects as defined herein, a method or use involves as step of detecting, counting, quantifying the level of, or measuring the concentration of blood eosinophil count, serum eosinophil cationic protein (ECP), serum tryptase and serum periostin. In some examples of aspects as defined herein, a method or use involves as step of detecting, quantifying the level of, or measuring the concentration of serum eosinophil cationic protein (ECP) and serum tryptase and optionally serum periostin.

The use of a combination of one or more SNP markers with one or more Th2 markers may in some cases improve the selection of patients. Thus is some examples of all aspects as defined herein, a method or use involves a step of detecting at least one SNP of the 17q12-21 region, and a step of quantification of at least one Th2 marker as defined herein, and using the result for selection of patients for treatment with immunotherapy products and/or predicting a response to treatment.

Thus, some examples of the aspects as defined herein, involves the combination of two or more biomarkers, wherein
a) At least one marker is a SNP of the 17q12-21 region, or a SNP in linkage disequilibrium with one or more SNPs of the 17q12-21 region as defined above is detected; and
b) At least one marker is a Th2 marker selected from the group consisting of blood eosinophil count, eosinophil cationic protein (ECP), tryptase, periostin, and total IgE is quantified.

Some examples of the aspects as defined herein, involves the combination of two or more biomarkers, wherein
a) At least one marker is a SNP of the 17q12-21 region, or a SNP in linkage disequilibrium with one or more SNPs of the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10) is detected; and
b) At least one marker is a Th2 marker selected from the group consisting of blood eosinophil count, eosinophil cationic protein (ECP), tryptase and periostin is quantified.

In some examples of the aspects as defined herein, a combination of two or more biomarkers is detected or quantified, wherein
a) At least one biomarker is the rs7216389 (SEQ ID NO: 10) T allele, or a SNP in linkage disequilibrium with one or more SNPs of the 17q12-21 region as defined above; and
b) At least one biomarker is a Th2 marker selected from the group consisting of blood eosinophil count, eosinophil cationic protein (ECP), tryptase and periostin.

In some preferred examples of the aspects as defined herein, a combination of two or more biomarkers is detected, wherein at least one biomarker is the rs7216389 (SEQ ID NO: 10) T allele or one or more SNPs in linkage disequilibrium with rs7216389 (SEQ ID NO: 10), and at least one biomarker is a Th2 marker selected from the group consisting of eosinophil count, eosinophil cationic protein (ECP), tryptase and periostin. In some more preferred examples a combination of two or more biomarkers is detected, wherein at least one biomarker is the rs7216389 (SEQ ID NO: 10) T allele, and at least one biomarker is a Th2 marker selected from the group consisting of blood eosinophil count, serum eosinophil cationic protein (ECP), serum tryptase and serum periostin.

### Immunotherapy Products

The aspects herein are related to the use of biomarkers in combination with one or more immunotherapy products, said immunotherapy products being administered to a subject in need thereof for preventing, reducing risk of, treatment and/or ameliorating symptoms and signs of atopic diseases or conditions, e.g. asthma. Immunotherapy products comprise an active ingredient, for example selected from the group consisting of antibodies (e.g. antibodies with an immunomodulatory effect), antibodies for treating asthma, allergens, modified allergens, allergoids, or fragments of allergens, probiotics or bacterial lysates.

The immunotherapy products provided herein are for use in the methods of treating, preventing, delaying onset, or reducing risk of an atopic disease, such as asthma, as defined herein. Thus, one example of an aspect of the invention provides an immunotherapy product for use in a method of treating, preventing or reducing risk of one or more atopic diseases, such as asthma, in a selected subject, said method comprising:
a) Detecting whether a biological sample obtained from said subject from said subject has one or more of:
   i. A single nucleotide polymorphisms (SNPs) selected from the group consisting of the single nucleic acid polymorphisms located in the chromosome 17q12-21 region and/or;
   ii. A SNP in linkage disequilibrium with one or more SNPs as defined in i. above;
b) Selecting and treating said subject with said immunotherapy product if one or more SNPs of section a) are identified in the biological sample from said subject.

Another example of an aspect of the invention provides an immunotherapy product for use in a method of treating, preventing or reducing risk of one or more atopic diseases, such as asthma, in a subject, said method comprising:
a) Obtaining or providing a biological sample from a subject;
b) Quantifying the level of one or more Th2 markers selected from the group consisting of eosinophils, eosinophil cationic protein (ECP), tryptase, periostin, and total IgE;
c) Selecting and treating said subject with an immunotherapy product and/or predicting a beneficial response to treatment with an immunotherapy product in said subject, if the level of one or more markers of step b) is increased or above a reference level.

When used in aspects as defined herein, antibodies (often referred to as biologics) can be polyclonal and monoclonal antibodies of any type and from any species, as well as immunoglobulin fragments such as Fv, Fab, Fab′, F(ab′)₂, or other antigen-binding antibody fragments, sequences or subsequences that interact with molecular specificity (e.g., demonstrate specific binding to with an antigen).

A number of antibodies have been developed for treatment of atopic disease, e.g. asthma. Thus, in some examples of aspects as defined herein, an immunotherapy product comprises an antibody useful for treatment of atopic disease, e.g. asthma. In some examples of aspects as defined herein, an immunotherapy product comprises an antibody selected from the group consisting of anti-IgE (e.g. Omalizumab), anti-IL4Rα (e.g. Dupilumab), anti-IL4 (e.g VAK694), anti-IL13 (e.g. Tralokinumab and Lebrikizumab), anti-IL5 (e.g. Mepolizumab and Reslizumab), anti-IL5 receptor (e.g. Benralizumab), anti-IL25, anti-thymic stromal lymphopoietin (TSLP) (e.g. Tezepelumab), anti-IL13/IL17 receptors (e.g. BITS7201A/RG7990) and anti-IL33 (e.g. AMG282).

Allergen-specific immunotherapy (AIT) is a form of immunotherapy applied in the treatment of IgE-mediated allergic or atopic diseases. Successful AIT is characterized by the development of clinical and immunological tolerance and modifies the response to allergen exposure. It has a disease-modifying effect, improves quality of life in allergic patients, as well as reduces symptoms and requirement for other allergy medication, like anti-histamines and steroids. Thus, in some preferred examples of the aspects disclosed herein, the immunotherapy product is a product for allergen-specific immunotherapy, i.e. an allergen-specific immunotherapy product.

In more specific examples of all aspects as defined herein, the allergen-specific immunotherapy product is used for preventing, reducing risk of, treatment or ameliorating of atopic disease, e.g. asthma, allergic rhinitis, allergic conjunctivitis or food allergy.

In essence, AIT involves the administration of an immunotherapeutic product (IT product) comprising one or more of the offending allergen(s) triggering the augmented IgE production. The offending allergens may be a single allergenic substance or a mixture of such substances and may be in the form of the wild-type allergen or a structurally modified form thereof. Optionally, the structurally modified form has reduced allergenicity, but maintained immunogenicity. Allergenicity is determined by the interaction of the B-cell epitopes of the allergens with the Fab region of the IgE antibody, while immunogenicity is the ability of an allergen to induce a humoral and/or cell-mediated immune response.

The wild-type allergen(s) may be provided in the form of extracts produced by gentle extraction of the natural occurring source of the offending allergen(s). Such extracts are termed natural allergen extracts. Alternatively, the wild-type allergen may be isolated from the natural source to obtain the wildtype in purified form, or it may be reproduced by recombinant engineering methods or synthetically.

A wide range of structurally modified allergens have been generated for the purpose of AIT, including at least:
a) Allergoid extracts that are chemically altered wild-type allergens, usually produced by treating the natural allergen extract with potassium cyanate, formaldehyde or glutaraldehyde to form conformational changes in the protein structure/function, which generally results in more limited capacity of IgE binding compared to the wild-type allergen.
b) Recombinant proteins which have an altered amino acid sequence compared to the wild-type allergen, for example oligomers, mutants, mosaic molecules, chimeric or hybrids, which has reduced IgE reactivity and retained T-cell reactivity, meaning the T-cell epitopes are essentially maintained in the altered amino acid sequence. Such recombinant proteins are often termed hypo-allergens.
c) T-cell epitope-containing peptides that are fragments of allergens comprising at least one T-cell epitope, which lack IgE antibody binding, but able to reduce allergic inflammation.

According to newer approaches, AIT may be conducted by administering plasmid DNA or RNA encoding the offending allergen or fragments of the allergens.

Thus, an immunotherapy product for the purpose of AIT may comprise an immunogenic agent selected from any one of; a natural allergen extract, an allergoid extract, a recombinant wild-type allergen, a recombinant hypo-allergen, B cell and/or T cell epitope-containing fragments of an allergen, a plasmid DNA encoding the allergen or fragments thereof, and mRNA encoding the allergen or fragments thereof. Thus, in particular examples of the aspects as defined herein, an allergen-specific immunotherapy product comprises one or more immunogenic agents selected from the group consisting of a natural allergen extract, an allergoid extract, a recombinant wild-type allergen, a recombinant hypo-allergen, B cell and/or T cell epitope-containing fragments of an allergen, a plasmid DNA encoding the allergen or fragments thereof, and mRNA encoding the allergen or fragments thereof. In more specific examples of the aspects as defined herein, an allergen-specific immunotherapy product comprises one or more immunogenic agents selected from the group consisting of natural allergen extract, allergoid extract and recombinant allergens.

Immunogenic agents, allergens, e.g. allergen extracts as described herein may be derived from or produced from a species selected from the group consisting of house dust mites (such as e.g. Dermatophagoides farinae, Dermatophagoides pterinussinus, and Blomia tropicalis), grass pollen (such as e.g. Pleum pretense pollen, Dactylus glomerate pollen, Lolium perenne pollen, Poa pratensis pollen), tree pollen (such as e.g. birch pollen, hazel pollen, alder pollen, hornbeam pollen, beech pollen, oak pollen, cedar pollen, ash pollen, olive pollen), weed pollen (such as e.g. ragweed pollen, mug wort pollen), insect venom, animals (such as e.g. cockroach, cat, dog or horse), and food species (such as e.g. peanut, egg, cow's milk, fish, wheat, soy, peach, kiwi, hazelnut, shellfish (such as e.g. crustaceans, mollusks)), moulds (such as e.g. Aspergillus fumigatus) and fungi (such as e.g. Malassezia species).

In particular examples of the aspects defined herein, allergen-specific immunotherapy is by administration of an allergen extract selected from the groups consisting of tree pollen allergen extract, weed pollen allergen extract, cedar pollen allergen extract, house dust mite allergen extract and grass pollen allergen extract to a subject in need thereof.

In other, more specific examples of the aspects defined herein, an allergen-specific immunotherapy product comprises a house dust mite allergen extract and/or a grass pollen allergen extract. In a more specific examples of the aspects defined herein, an allergen-specific immunotherapy product comprises a house dust mite allergen. In another, more specific example of the aspects defined herein, an allergen-specific immunotherapy product comprises a grass pollen allergen extract.

Thus, in some specific examples of one aspect of the invention, the invention provides an allergen specific immunotherapy product (e.g. comprising house dust mite allergens) for use in a method of treating, preventing or reducing risk of asthma, in a selected subject, said method comprising:
a) Detecting whether a biological sample obtained from said subject from said subject has one or more of:
   i. A single nucleotide polymorphisms (SNPs) selected from the group consisting of the single nucleic acid polymorphisms located in the chromosome 17q12-21 region (such as one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10), or complementary SNPs thereof) and/or;
   ii. A SNP in linkage disequilibrium with one or more SNPs as defined in i) above;
b) Selecting and treating said subject with said allergen specific immunotherapy product if it is detected that the subject is a homozygote of one or more SNPs of section a).

Another specific example of an aspect of the invention provides an allergen specific immunotherapy product (e.g. comprising house dust mite allergens) for use in a method of treating, preventing or reducing risk of asthma, in a subject, said method comprising:
a) Obtaining or providing a biological sample from a subject;
b) Quantifying the level of one or more Th2 markers selected from the group consisting of eosinophils, eosinophil cationic protein (ECP), tryptase, and periostin;
c) Selecting and treating said subject with an immunotherapy product and/or predicting a beneficial response to treatment with an immunotherapy product in said subject, if the level of one or more (e.g. two or more) markers of step b) is increased or above a reference level.

Immunotherapy products as described in all examples herein, may comprise excipients. An excipient is generally a pharmaceutically inactive substance formulated with the active ingredient (API) of a medication. Excipients are commonly used to bulk up formulations that contain potent active ingredients (thus often referred to as “bulking agents,” “fillers,” or “diluents”), to allow convenient and accurate dispensation of a drug substance when producing a dosage form. In one example of the aspects as defined herein, an immunotherapy product comprises one or more excipients. Said one or more excipients may act as a solid carrier, diluent, flavouring agent, solubilizer, lubricant, glidants, suspending agent, binder, filler, preservative, antiadherents, wetting agent, tablet disintegrating agent, sorbent, and/or an encapsulating/coating material.

In aspects as defined herein, an immunotherapy product is administered to individuals in need of treatment in therapeutically effective doses. A therapeutically effective amount of an immunotherapy product is an amount sufficient to treat, cure, prevent, reduce the risk of, alleviate or partially arrest the clinical manifestations (i.e. signs and symptoms) of a given atopic disease or disorder (e.g. asthma) and its complications. The amount that is effective for a particular therapeutic purpose will depend on the severity and the sort of the atopic disorder as well as on the weight and general state of the subject.

Immunotherapy products as used in aspects herein may generally be formulated for any suitable administration route. It will be appreciated that the preferred route of administration of an immunotherapy products as defined herein will depend on the general condition and age of the subject to be treated, the nature of the condition to be treated, the location of the tissue to be treated in the body and the active ingredient chosen.

Such routes of administration are any suitable routes, such as an enteral route, the oral, rectal, nasal, pulmonary, buccal, sublingual, transdermal, intracisternal, intraperitoneal, and parenteral (including subcutaneous, intradermal, intramuscular, intrathecal, intravenous and intrathecal) routes. In general, dosage forms for such administration of an immunotherapy product as described herein may be prepared by suitable techniques known in the art.

In more specific examples of the aspects as defined herein, the immunotherapy product is administered by parental administration, such as by subcutaneous, intradermal, intraveneous or intrapulmonary injection.

In other specific examples of the aspects as defined herein, the immunotherapy product is administered into the airways, such as by inhalation or nebulization.

Oromucosal administration, such as subligual administration is typically used for allergen-specific immunotherapy. Thus, in other examples of the aspects as defined herein, the immunotherapy product (e.g. a allergen-specific immunotherapy product) is administered by oromucosal administration, such as buccal or sublingual administration.

For oromucosal administration, such as sublingual administration, an immunotherapy product may for example be formulated as a tablet, or a liquid. Thus, in some examples of aspects as defined herein, the immunotherapy product is for allergen-specific immunotherapy by sublingual administration of one or more tablets comprising an allergen extract to a subject.

Such tablets may be formulated for fast up-take, e.g. such as a fast dispersing tablet. In other examples of aspects as defined herein, the immunotherapy product is for allergen-specific immunotherapy by sublingual administration of a liquid formulation of an allergy extract to a subject.

In a more specific example of the aspects as defined herein, the immunotherapy product is for sublingual allergen-specific immunotherapy and/or sub-cutaneous allergen-specific immunotherapy. In some more specific examples, allergen-specific immunotherapy involves oromucosal administration of a product comprising house dust mite allergen extract to a subject in need, such as sublingual administration of house dust mite allergen extract to a subject in need. In other more specific examples, allergen-specific immunotherapy comprises oromucosal administration of a product comprising grass pollen allergen extract to a subject in need, such as sublingual administration of grass pollen allergen extract to a subject in need.

The immunotherapy produces when used in methods of treatment as defined herein, may be administered in combination with other active ingredients for treatment of asthma, e.g. by separate, simultaneous or sequential administration. Thus in some examples of the aspects of the invention, an immunotherapy product is administered in combination with at least one second active ingredient useful for treatment of asthma.

In one example of the aspects of the invention, an allergen-specific immunotherapy product is for example administered in combination with an antibody useful for treatment of asthma, such as selected from the group consisting of anti-IgE (e.g. Omalizumab), anti-IL4Rα (e.g. Dupilumab), anti-IL4 (e.g VAK694), anti-IL13 (e.g. Tralokinumab and Lebrikizumab), anti-IL5 (e.g. Mepolizumab and Reslizumab), anti-IL5 receptor (e.g. Benralizumab), anti-IL25, anti-thymic stromal lymphopoietin (TSLP) (e.g. Tezepelumab), anti-IL13/IL17 receptors (e.g. BITS7201A/RG7990) and anti-IL33 (e.g. AMG282), optionally in combination with further active ingredients for treatment of asthma. In one example of aspects as defined herein, the immunotherapy product is for allergen-specific immunotherapy, and is administered in combination with an an anti-IL4Rα antibody, e.g. Dupilumab.

In one example of the aspects of the invention, an immunotherapy product (e.g. an allergen-specific immunotherapy product) is administered in combination with one or more second agents for asthma treatment selected from the group consisting of corticosteroids (inhaled, systemic and/or oral), β₂-adrenergic agonists (short acting or long acting), muscarinic receptor antagonists (e.g. long acting muscarinic receptor antagonists, LAMA), anticholinergics, mast cell stabilizers, leukotriene antagonists (e.g. motelukast sodium) or leukotriene modifiers, cromolyn sodium and methylxanthines.

In one example of the aspects of the invention, an immunotherapy product (e.g. an allergen-specific immunotherapy product) is administered in combination with inhaled corticosteroid (e.g. budesonide, fluticasone propionate, fluticasone furoate or beclomethasone) and optionally a short acting δ₂-adrenergic agonist (e.g. salbutamol or terbutaline).

In one example of the aspects of the invention, an immunotherapy product (e.g. an allergen-specific immunotherapy product) is administered in combination with inhaled corticosteroid (e.g. budesonide) and optionally a short acting δ₂-adrenergic agonist (e.g. salbutamol).

In one example of the aspects of the invention, an immunotherapy product (e.g. an allergen-specific immunotherapy product) is administered in combination with inhaled corticosteroid (e.g. budesonide, fluticasone propionate, fluticasone furoate or beclomethasone) and a long acting β₂-adrenergic agonist (e.g. salmeterol, formeterol, or vilanterol) and optionally a short acting β₂-adrenergic agonist (e.g. salbutamol or terbutaline).

In one example of the aspects of the invention, an immunotherapy product (e.g. an allergen-specific immunotherapy product) is administered in combination with inhaled corticosteroid (e.g. budesonide) and a long acting β₂-adrenergic agonist (e.g. salmeterol, formeterol, or vilanterol) and optionally a short acting β₂-adrenergic agonist (e.g. salbutamol).

In one example of the aspects of the invention, an allergen-specific immunotherapy product comprising house dust mite allergen extraxt is administered in combination with inhaled corticosteroid (e.g. budesonide) and a long acting β₂-adrenergic agonist (e.g. salmeterol, formeterol, or vilanterol) and optionally a short acting β₂-adrenergic agonist (e.g. salbutamol).

In more specific examples of the aspects as defined herein, the immunotherapy product is for sublingual administration of a house dust mite allergen extract to a subject in need thereof, and is administered in combination with a inhaled corticosteroid (e.g. budesonide) and optionally a β₂-adrenergic agonist (e.g. salbutamol).

### Atopic Disease

Atopic disease involves sensitization to one or more allergens, and/or the presence of detectable IgE in response to common environmental proteins or other allergens. Sensitization to one or more allergens can be measured by use of a skin prick test, wherein as small amount of allergen is administered just below the skin surface. The area of the skin where the allergen is administered is observed for a time period of e.g. about 15 minutes to see if a reaction develops. The “wheal” (a raised, red, itchy bump and surrounding “flare”) indicates sensitivity to the given allergen. The larger the wheal and flare, the greater the sensitivity. Additionally, the presence of IgE to an allergen can be detected using conventional immunoassay methods such as for example Immunocap (Thermo Scientific), microarrays or other methods, wherein allergen-specific IgE is detected and/or measured. When using an Immunocap method, typically a detection of IgE at a level above 0.35 kU/L in a biological sample (e.g. blood serum) is associated with atopic disease or allergy.

The presence of atopy in an individual is associated with an increased risk of developing one or more of the atopic diseases or conditions such as atopic dermatitis, asthma, allergic rhinitis, allergic conjunctivitis or allergic rhinoconjunctivitis, food allergy, anaphylaxis, anaphylactic shock, allergic bronchopulmonary aspergillosis, urticaria, itch and hives.

In some examples of the aspects as defined herein, atopic disease is selected from the group consisting of atopic dermatitis, asthma, allergic rhinitis, allergic conjunctivitis and allergic rhinoconjunctivitis, more preferably selected from the group consisting of asthma, allergic rhinitis, allergic conjunctivitis and allergic rhinoconjunctivitis. In a more preferred example, atopic disease is asthma.

Atopic dermatitis, also known as atopic eczema, is a type of inflammation of the skin. Atopic dermatitis is often accompanied by one or more of the signs and symptoms selected from the group consisting of dry skin, itching of the skin (e.g. typically more pronounced at night), red to brown or gray patches of the skin which are typically on the hands, feet, ankles, wrists, neck, upper chest, inside the bend of elbows and/or knees, face and/or scalp, small raised bumps on the skin, which may leak fluid, thickened, cracked, scaly skin, and raw, sensitive or swollen skin.

Allergy, often manifested as allergic rhinitis and/or allergic conjunctivitis, is often accompanied by one or more of the signs and symptoms selected from the group consisting of sneezing, itching (of nose, eyes, ears, palate), rhinorrhea (runny nose), postnasal drip, congestion, anosmia, headache, earache, tearing, red eyes, eye swelling, coughing, fatigue, drowsiness, and malaise. Other types of allergy includes food allergy, which is often accompanied by one or more of the signs and symptoms selected from the group consisting of rash, hives, itching of mouth, lips, tongue, throat, eyes, skin, or other areas, swelling (angioedema) of lips, tongue, eyelids, or the whole face, difficulty swallowing, runny and/or congested nose, hoarse voice, wheezing and/or shortness of breath, diarrhea, abdominal pain, and/or stomach cramps, lightheadedness, fainting, nausea and vomiting.

Asthma is a common long-term inflammatory disease of the airways of the lungs. It is characterized by variable and recurring symptoms, reversible airflow obstruction, and bronchospasm. Asthma is often accompanied by one or more of the signs and symptoms selected from the group consisting of wheezing (e.g. a whistling, squeaky sound during breathing), shortness of breath, chest tightness, rapid breathing, increased coughing, fatigue, nocturnal awakening and/or problems sleeping (for example due to coughing or difficulty breathing), decreased lung function, such as decreased peak expiratory flow (PEF), decreases forced expiratory volume in one second (FEV1), or such as decreased forced vital capacity (FVC), or decreased ratio of FEV1 compared to FVC (FEV1/FVC), increased fraction of exhaled nitric oxide (FeNO), and increased number of eosinophils in blood, and/or sputum. These episodes, wherein a subject may experience one or more symptoms and signs of asthma, may occur at a differing frequency in different subjects, such as for example a few times a day or a few times per week. Further, depending on the subject, experience of one or more symptoms or signs of asthma may be more frequent at different time points during the circadian cycle, (e.g. more frequent during the night, or when waking up), or at the occurrence of specific events (e.g. viral infections, exercise, laughing, crying, changes in weather, cold air, car exhaust fumes, smoke or strong smells).

Allergic asthma is an asthma phenotype which is often, but not always, associated with a past and/or family history of allergic disease such as atopic dermatitis, allergic rhinitis, allergic conjunctivitis, food allergy and/or drug allergy. Allergic asthma often commences in childhood, but may also commence later in life. In some examples of the aspects as defined herein, asthma is allergic asthma, i.e. the subject is sensitive to or has IgE which specifically binds one or more allergens, e.g. one or more house dust mite allergens, i.e. group 1 and/or group 2 allergens of house dust mites, such as Der p 1, Der p 2, Der f 1 and/or Der f 2. An atopic disease, such as asthma, such as allergic asthma may be caused by exposure to house dust mites.

Often, asthma is classified according to the age where the first symptoms and signs of asthma occurred for the first time in a subject, or when a subject was first diagnosed with asthma. Early on-set or childhood asthma is typically used to denote asthma where the subject has beend diagnosed with and/or experienced symptoms and signs of asthma in childhood. In some examples of the aspects as defined herein, asthma is childhood and/or early on-set asthma, and the subject has beend diagnosed with and/or experienced symptoms and signs of asthma in childhood, such as before the age of 12 years, for example before the age of 5 years, e.g. at the age of 1, 2, 3 or 4 years, or such as before the age of 8 years, e.g. at the age of 5, 6 or 7 years, or such as before the age of 12 years, e.g. at the age of 8, 9, 10 or 11 years, or such as before the age of 18 years.

In some examples of the aspects as defined herein, asthma is allergic asthma, and the subject has been diagnosed with and/or experienced symptoms and/or signs of asthma in childhood, such as before the age of 12 years.

Asthma may further be classified according to the severity based on the occurrence, persistence, and specific symptoms and signs of asthma, as well as the treatment modalities needed to treat the asthma exacerbation and/or reduce, abrupt or ameliorate signs and symptoms of asthma. Asthma classes include intermittent asthma, mild (persistent) asthma, moderate (persistent) asthma, severe (persistent) asthma and refractory asthma.

The term intermittent asthma is used in cases wherein wheezing and coughing is observed or experienced no more than 2 days a week, and/or where nighttime exacerbations occur twice a month at most, and where the subject is free of asthma symptoms outside of these few exacerbations.

Mild (persistent) asthma is used to denote asthma cases where symptoms occur more than twice a week but less than once a day, and exacerbations may affect activity (such as exercise), and/or where nighttime exacerbations occur more often than twice a month but less than once a week. In such cases, lung function measured in FEV1 predicted or FEV1/FVC is often about 80% of normal or greater.

Moderate (persistent) asthma is used to denote asthma cases where symptoms of asthma occur daily. Typically, asthma exacerbations in this type of asthma occur daily and may last for several days. Coughing and wheezing may disrupt the subject's normal activities and make it difficult to sleep. Nighttime exacerbations may occur more than once a week. In such cases, lung function is reduced, for example FEV1 predicted may be between about 60% to about 80% and/or FEV1/FVC may be between about 75% to about 80%.

Severe (persistent) asthma is used to denote asthma cases where symptoms of asthma occur daily throughout the day. As a result, daily activity is very limited. Depending on the age, the subject may experience night time awakening one or more times during a week, typically about 5, 6 or 7 times per week. In such cases, lung function is reduced, for example FEV1 predicted may be less than about 60% and/or FEV1/FVC may be less than about 75%. In some examples of severe asthma, called (severe) refractory asthma, asthma symptoms and signs cannot be treated or ameliorated using conventional treatment, such as ICS with our without systemic corticosteroid, and severe asthma excerbations occur about 2 or more times per year (see Bel et al 2010).

In some examples of aspects as defined herein, asthma is selected from one or more of the group of asthma types consisting of intermittent asthma, mild (persistent) asthma, moderate (persistent) asthma, severe (persistent) asthma and refractory asthma. In other, more specific examples of aspects as defined herein, asthma is one or more types of asthma selected from the group consisting of moderate asthma, severe asthma and refractory asthma. In still other examples of aspects as defined herein, asthma is one or both types of asthma selected from the group consisting of moderate and severe asthma. In some examples of aspects as defined herein, asthma is moderate and/or severe allergic asthma.

Asthma exacerbations, asthma attacks or asthma “flare-ups” consist of acute or subacute episodes, wherein the airways become swollen and inflamed. Further, the muscles around the airways contract and the airways also produce extra mucus, which causes the bronchial tubes to narrow. An asthma exacerbation can be observed by a worsening of one or more symptoms and signs of asthma. In some examples of the aspects defined herein, asthma exacerbations is worsening of two or more symptoms and signs of asthma, such as for example a worsening of 2, 3, 4, 5, 6, 7 or more symptoms and/or signs of asthma. In some examples of the aspects as defined herein, an asthma exacerbation is worsening in at least two of the symptoms selected from the group consisting of shortness of breath, coughing, wheezing, and chest tightness.

Asthma exacerbations may be grouped according to the severity based on the occurrence, persistence, increase of, specific symptoms and signs of asthma, as well as the amount, frequency and treatment modalities needed to treat the asthma exacerbation and/or reduce, abrupt or ameliorate signs and symptoms of asthma. When increase is symptoms, or medication is used to distinguish between the different severity types of asthma exacerbations, a comparison is typically made to baseline, or normal values.

Baseline values, or normal values of, for example use of inhaled corticosteroids (ICS) and/or short acting β2-agonist (SABA), as well as asthma symptoms score may depend on an individual subject, or be calculated by averaging over a group of subjects suffering from asthma, for example a group of subjects of the same age, gender, of suffering from the same type of asthma (e.g. allergic asthma), or the same degree of severity of asthma.

A mild asthma exacerbation, is typically a relatively small increase in symptoms and signs of asthma above baseline and depends on the individual subject.

The need for increased used of SABA may to treat asthma exacerbation is typically used to define certain examples of asthma exacerbations. In some examples of aspects as defined herein, a mild asthma exacerbation may involve a need of treatment with SABA for one day, or night, or may involve increased dose of SABA treatment during one day or night. In some examples of aspects as defined herein, a mild asthma exacerbation does not require SABA treatment, or increase in SABA dose, in two consecutive days or nights.

In some examples of the aspects as defined herein, a moderate asthma exacerbation typically involve a need of treatment with SABA for at least two consecutive days or at least two consecutive nights, such as at least 2, 3, 4, 5, 6, 7 or more days or nights. In some examples of the aspects as defined herein, a moderate asthma exacerbation is when the need for SABA may be further increased by the number of daily administrations needed, for example such as an increase of 3, 4, 5, 6, 7 or more doses or puffs of SABA per day. In some examples of the aspects as defined herein, a moderate asthma exacerbation involves an increase from the baseline value in occasions of SABA use on at least 2 consecutive days (e.g. a minimum increase of 4 puffs per day).

The need for hospitalization, or visit to emergency room, to treat asthma exacerbation may further be used to define certain types of asthma exacerbations. In some examples of aspect as defined herein, a moderate asthma exacerbation requires hospitalization or emergency treatment, but does not require the use of systemic corticosteroids. In some examples of severe asthma exacerbations, hospitalization for more than 12 hours and/or emergency room visit is required for treatment.

The degree of reduction in lung function may further be used to define certain types of asthma exacerbations. In some examples of the aspects as defined herein, a mild asthma exacerbation is an exacerbation wherein a reduction of <about 20% in FEV1 is observed compared to baseline, or a previous measurement, and/or wherein a a reduction of <about 20% in PEV is observed compared to baseline, or a previous measurement.

In some examples of the aspects as defined herein, moderate asthma exacerbation may is an exacerbation wherein a reduction of about ≥20% in FEV1 is observed compared to baseline, or a previous measurement, and/or wherein a a reduction of ≥ about 20% in PEV is observed compared to baseline, or a previous measurement. The reduction in lung function may be further observed over one or more days, such as for example over at least 2 consecutive days, such as over 2, 3, 4, 5, 6, 7 or more consecutive days.

The need for use of systemic corticosteroids (e.g. oral corticosteroids (OCS)) to treat an asthma exacerbation is typically used to define certain examples of asthma exacerbations. In some examples of the aspects as defined herein, a severe asthma exacerbation involve the need of treatment with systemic corticosteroid. In one example of the aspects as defined herein, a severe asthma exacerbation typically involve a need of treatment with systemic corticosteroids for at least 3 days and/or emergency room visit due to asthma requiring systemic corticosteroids or hospitalization for more than 12 hours because of asthma.

In some examples of the aspects as defined herein, a moderate asthma exacerbation may be defined using one or more of the criteria a) to d) below:
a) Nocturnal awakening(s) due to asthma requiring SABA use for at least 2 consecutive nights or an significant increase of in asthma symptoms from baseline value (e.g. a minimum 0.75 in daily symptom score) on at least 2 consecutive days.
b) An increase from the baseline value in occasions of SABA use on at least 2 consecutive days (a minimum increase of 4 puffs per day).
c) ≥20% decrease in peak expiratory flow (PEF) from baseline value on at least 2 consecutive mornings or evenings or a ≥20% decrease in FEV1 from baseline value.
d) Visit to the emergency room or unscheduled visit to a medical centre for asthma treatment not requiring systemic corticosteroids.

In some examples of the aspects as defined herein, a severe asthma exacerbation may be defined using one or two the criteria e) to f) below:
e) Need of systemic corticosteroids for the treatment of asthma symptoms for at least 3 days.
f) Emergency room visit because asthma requiring systemic corticosteroids or hospitalization for more than 12 hours because of asthma.

In one specific example of an aspect of the invention a method for selecting a subject for treatment with an immunotherapy product for treatment of asthma and/or predicting response to treatment with an immunotherapy product for treatment of asthma is provided, comprising:
a) Detecting whether a biological sample obtained from said subject from said subject has one or more of:
   i. A single nucleotide polymorphisms (SNPs) selected from the group consisting of the single nucleic acid polymorphisms located in the chromosome 17q12-21 region (such as one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10), or complementary SNPs thereof) and/or;
   ii. A SNP in linkage disequilibrium with one or more SNPs as defined in i) above;
b) Selecting and treating said subject with said allergen specific immunotherapy product if it is detected that the subject is a homozygote of one or more SNPs of section a).

Another specific example of an aspect of the invention provides a method of treating, preventing or reducing risk of asthma, in a subject, said method comprising:
d) Obtaining or providing a biological sample from a subject;
e) Quantifying the level of one or more Th2 markers selected from the group consisting of eosinophils, eosinophil cationic protein (ECP), tryptase, and periostin;
f) Selecting and treating said subject with an allergen-specific immunotherapy product (e.g. comprising one or more house dust mite allergens) and/or predicting a beneficial response to treatment with an allergen-specific immunotherapy product (e.g. comprising one or more house dust mite allergens) in said subject, if the level of one or more (e.g. two or more) markers of step b) is increased or above a reference level.

Asthma may be treated using a number of different other secondary agents (or active ingredients) selected from the group consisting of corticosteroids (inhaled, systemic, oral or injected), β2-adrenergic agonists (such as short acting β2-adrenergic agonists (SABA) and long acting β2-adrenergic agonists (LABA) and anti-cholinergic agents (e.g. tiotropium), mast cell stabilizers, leukotriene antagonists or leukotriene modifiers, cromolyn sodium and/or methylxanthines.

In preferred examples of the aspects as defined herein, asthma is treated using at least corticosteroids (inhaled, systemic, oral or injected) and optionally β2-adrenergic agonists, such as using inhaled corticosteroids and optionally a short-acting β2-adrenergic agonists, e.g. inhaled budesonide, optionally in combination with salbutamol.

### Response to Treatment

Treatment with immunotherapy products as defined herein may result in different responses depending on the subject and related type of atopic disease, age, genetic heritage, and environment.

Desirable, or beneficial effects of treatment with (or response to) immunotherapy products include preventing or delaying occurrence (e.g first occurrence, or recurrence) of atopic disease, alleviation of symptoms, diminishment of any direct or indirect pathological consequences of atopic disease, decreasing the rate of atopic disease progression, amelioration or palliation of the atopic disease state, and remission or improved prognosis. Thus, in some examples of the aspects as defined herein, the treatment with immunotherapy products as defined herein, has beneficial effect on one or more atopic diseases.

In some examples of the aspects as defined herein, the use of immunotherapy products is to obtain a response or effect selected from the group consisting of preventing, reducing risk of, treatment, ameliorating symptoms and signs of one or more atopic diseases. In more examples of the aspects as defined herein, the use of immunotherapy products is to obtain a response or effect selected from the group consisting of preventing, reducing risk of, treatment, ameliorating symptoms and signs of one or more atopic diseases selected from the group consisting of atopic dermatitis, asthma, allergic rhinitis, allergic conjunctivitis and allergic rhinoconjunctivitis, more preferably selected from the group consisting of asthma, allergic rhinitis, allergic conjunctivitis and allergic rhinoconjunctivitis.

As mentioned above, asthma is a disease which often affects the life of a subject considerably, and which may even have fatal outcome. Thus, in some examples of the aspects as defined herein, the use of immunotherapy products (e.g. allergen-specific immunotherapy) is to obtain a response selected from the group consisting of preventing, reducing risk of, treatment, ameliorating symptoms and signs of (at least) asthma. In some more specific examples of the aspects as defined herein, the use of an immunotherapy product (e.g. allergen-specific immunotherapy) is to reduce risk of asthma exacerbations, such as increasing the time to occurrence of a moderate and/or severe asthma exacerbation, and/or reduce risk of deterioration of one or more symptoms and signs of asthma.

In some more specific examples of aspects as defined herein, the beneficial response to treatment with immunotherapy treats, ameliorates, or reduces risk of one or more of the signs and symptoms of asthma selected from the group consisting of wheezing (e.g. a whistling, squeaky sound during breathing), shortness of breath, chest tightness, rapid breathing, increased coughing, fatigue, nocturnal awakening and/or problems sleeping (for example due to coughing or difficulty breathing), decreased lung function, such as decreased peak expiratory flow (PEF), decreases forced expiratory volume in one second (FEV1), or such as decreased forced vital capacity (FVC), or decreased ratio of FEV1 compared to FVC (FEV1/FVC), increased fraction of exhaled nitric oxide (FeNO), and increased number of eosinophils in blood, and/or sputum.

Asthma patients whose symptoms can usually be rapidly relieved by inhaled ICS and/or SABA, may still be at risk of experiencing a severe asthma exacerbation or death due to asthma. Therefore it is of high importance to identify treatments and/or methods of treatment which are capable of reducing the risk of more severe asthma exacerbations. Thus in some, more specific examples of the aspects as defined herein, a response to treatment with immunotherapy (e.g. allergen-specific immunotherapy) is reducing the risk, rate or frequency, of moderate to severe asthma exacerbations.

Further, since asthma may influence a subject's daily life and activity, it further beneficial to identify treatments and/or methods of treatment which are capable of reducing risk of experiencing one or more asthma signs and symptoms. Thus, in some specific examples of the aspects as defined herein, a (beneficial) response to treatment is a reduction in risk of experiencing, and/or ameliorating of one or more asthma signs and symptoms. In some examples of the aspects as defined herein, a (beneficial) response to treatment is a reduction of the use of use of asthma medication, e.g. ICS, SABA, OCS or any combinations of these medications. In some more examples of the aspects as defined herein, a (beneficial) response to treatment is a reduction in the need for use of asthma medication selected from the group consisting of inhaled, systemic or oral corticosteroids. In some more examples of the aspects as defined herein, a (beneficial) response to treatment is a reduction in the need for use of β₂-adrenergic agonists (SABA). In some more examples of the aspects as defined herein, a (beneficial) response to treatment is a reduction in the need for use of inhaled corticosteroid in combination with β₂-adrenergic agonists (SABA). In some more examples of the aspects as defined herein, a (beneficial) response to treatment is a reduction in the need for use of systemic corticosteroid (e.g. OCS).

In some examples of the aspects as defined herein, a (beneficial) response to treatment is a reduced risk of asthma exacerbations and/or deterioration in asthma symptoms.

Subjects to be Treated with Immunotherapy

The aspects of the invention relate to identification and/or selection of patients or “subjects in need” that may benefit from administration of immunotherapy products. In some examples of the aspects as defined herein, a subject in need has one or more SNPs of the 17q12-21 chromosomal region, as defined above.

A subject in need of treatment with an immunotherapy product in aspects as defined herein may or may not have been diagnosed with, or have experienced symptoms of, one or more atopic diseases, e. g. asthma. In some examples of the aspects defined herein, the subject has been diagnosed with and/or experienced symptoms of one or more atopic diseases selected from the group consisting of atopic dermatitis, allergic rhinitis, allergic conjunctivitis, food allergy and asthma. In some examples of the aspects defined herein, the subject has been diagnosed with and/or experienced symptoms of allergic rhinitis and/or allergic conjunctivitis.

In some examples of the aspects as defined herein, the subject has experienced symptoms and/or signs of asthma, or has previously been diagnosed with asthma. Thus, in some examples of the aspects as defined herein, the subject has experienced symptoms and/or signs of asthma, or has previously been diagnosed with asthma, and further has one or more other atopic disease, for example such as allergic rhinitis and/or allergic conjunctivitis.

In other examples of the aspects as defined herein, subject has not previously been diagnosed with asthma, or experienced one or more symptoms of asthma. Thus, in some examples of the aspects as defined herein, the subject has not experienced symptoms and/or signs of asthma, or has not previously been diagnosed with asthma, and further has one or more other atopic disease, for example such as allergic rhinitis or allergic conjunctivitis.

As mentioned above, asthma can be classified into different phenotypes, depending on the age where the symptoms of disease is first observed and/or the disease is diagnosed. Thus, in some examples of the aspects defined herein, the subject has, or has been diagnosed with and/or experienced symptoms of early on-set asthma.

Further, as mentioned above, asthma can be classified into different degrees of severity. Thus, is some examples of the aspects as defined herein, the subject has, or has previously been diagnosed with and/or experienced symptoms of one or more degrees of severity of asthma selected from the group consisting of intermittent asthma, mild asthma, moderate asthma, severe asthma and refractory asthma. In some examples of the aspects as defined herein, the subject has previously been diagnosed with and/or experienced symptoms of intermittent and/or mild asthma.

In some examples of the aspects as defined herein, the subject has previously been diagnosed with and/or experienced signs and symptoms of one or more types of asthma selected from the group consisting of moderate asthma, severe asthma, and refractory asthma.

In some even more preferred examples of the aspects as defined herein, the subject has previously been diagnosed with and/or experienced signs and symptoms of moderate asthma and/or severe asthma, such as moderate asthma, or such as severe asthma.

In some even more preferred examples of the aspects as defined herein, the subject has previously been diagnosed with and/or experienced signs and symptoms of moderate allergic asthma and/or severe allergic asthma, such as moderate allergic asthma, or such as severe allergic asthma.

In some examples of the aspects as defined herein, the subject has previously been diagnosed with and/or experienced signs and symptoms of a moderate and/or severe asthma exacerbation, such as a moderate asthma exacerbation, or such as a severe asthma exacerbation.

In some examples of the aspects as defined herein, the subject has not previously been diagnosed with and/or experienced signs and symptoms of a moderate and/or severe asthma exacerbation, such as a moderate asthma exacerbation, or such as a severe asthma exacerbation.

In some examples of the aspects as defined herein, the subject is, or has previously been, diagnosed with and/or experienced symptoms of allergy against one or more allergens. Thus in some examples of aspects as defined herein, the subject is, or has previously been, diagnosed with allergy by detection or measurement of specific IgE to one or more allergens, and/or by a positive skin prik test using one or more allergens. In some, even more specific examples of the aspects as defined herein, the subject is or has been diagnosed with at least house dust mite allergy.

A concept behind AIT is that a subject is treated by administration of one or more specific allergens that the given subject is allergic against. Thus, in some examples of the aspects as defined herein, the subject is allergic to, has experienced symptoms of, or has been diagnosed with, allergy or sensitivity to one or more of the allergens comprised in the allergen-specific immunotherapy product used for treatment.

In some examples of the aspects as defined herein, the subject is sensitive to, or has allergy to house dust mite, and the allergen immunotherapy is by administration of a one or more house dust mite allergens, (e.g. house dust mite allergen extract) to the subject. Thus, in some more specific examples of the aspects as defined herein, the subject has house dust mite allergy, such as house dust mite-related allergic rhinitis or house dust mite-related allergic conjunctivitis, and the allergen immunotherapy is by administration of one ore more house dust mite allergens, (e.g. a house dust mite allergen extract) to the subject. In some other examples of the subject is sensitive to, or has allergy to grass pollen allergens, and the allergen immunotherapy is by administration of a one or more grass pollen allergens, (e.g. grass pollen allergen extract) to the subject. Thus, in some more specific examples of the aspects as defined herein, the subject has grass pollen allergy, such as grass pollen-related allergic rhinitis or grass pollen-related allergic conjunctivitis, and the allergen immunotherapy is by administration of one or more grass pollen allergens (e.g. a grass pollen allergen extract) to said subject.

In some, more preferred examples of aspects as defined herein the subject has been diagnosed with and/or experienced symptoms of allergic asthma. In some, more preferred examples of aspects as defined herein the subject has been diagnosed with and/or experienced symptoms of moderate and/or severe allergic asthma.

In some, more preferred examples of the aspects as defined herein, a the subject has been diagnosed with and/or experienced symptoms of moderate and/or severe allergic asthma, and has one or more types of allergy, e.g. house dust mite allergy.

Immunotherapy products as mentioned above, (and in particular AIT products) have the ability to modify the immuneresponse, and in some cases prevent progression of atopic disease (e.g progression from allergy into allergic asthma). Since genetic markers (e.g. SNPs of the chromosome 17q12-21 region) are inherently present, even before the development of, or progression of, atopic disease, it may be possible to have a beneficial effect of treatment with immunotherapy products as defined herein at a point in time where the subject has not experienced symptoms of, or been diagnosed with, one or more atopic diseases. Thus in some examples of the aspects as defined herein, the subject has not experienced symptoms of, or been diagnosed with an atopic disease selected from the group consisting of atopic dermatitis, allergic rhinitis, allergic conjunctivitis, food allergy and asthma. In some more examples of the aspects as defined herein, the subject has not experienced symptoms or signs of, or has not been diagnosed with asthma. In some examples of the aspects as defined herein, the subject has not been not previously experienced an asthma exacerbation selected from the group consisting of a mild asthma exacerbation, a moderate asthma exacerbation and a severe asthma exacerbation. In a more specific example of aspects as defined herein, the subject has not previously experienced an asthma exacerbation selected from the group consisting of a moderate asthma exacerbation and a severe asthma exacerbation.

Genetics play a large role in developing atopic diseases, these underlying genetic risks often react to a trigger in the environment to cause the atopic disease. Close relatives, such as for example parents and their children, often share the same environment to a large degree. Therefore, if a close relative of a subject has been diagnosed with, or experienced signs and symptoms of atopic disease, there is an increased risk that the subject will also experience signs and symptoms of atopic disease. Thus, in some examples of the aspects as defined herein, one or more close relatives of a subject has been diagnosed with and/or experienced symptoms of atopic disease, such as atopic dermatitis, allergy and/or asthma. In some examples of aspects as defined herein one or more parent(s) of the subject has been diagnosed with and/or experienced symptoms of atopic disease, such as allergy and/or asthma. In some examples of aspects as defined herein, one or more parent(s) of the subject has been diagnosed with and/or experienced symptoms and signs of moderate to severe atopic disease, such as moderate to severe allergy and/or moderate to severe asthma.

Genetic biomarkers, e.g. SNPs of the 17q12-21 chromosomal region as defined herein may be associated with atopic disease (e.g. asthma) to a varying degree in subjects having a different descent, ancestry or genetic heritage. Similarly, SNPs of the 17q12-21 chromosomal region as defined herein may be associated with treatment effect of immunotherapy products on atopic disease, such as asthma, to a varying degree depending on the ancestry or descent of the subject. In some examples of the aspects defined herein, a subject in need is of European, Hispanic (latino) and/or Asian descent, such as of European ancestry, Latino ancestry, and/or Asian ancestry. In other examples of the aspects defined herein, a subject in need is not of African or Afro American descent or ancestry. In specific examples of the aspects defined herein, a subject is of Caucasian ancestry or European descent.

A subject in need as defined herein above, may have differing age and gender. Some examples of subjects of different ages include adults, adolescents and children. In one specific example of the aspects as defined herein, the subject is an adult, e.g. an adult above 17 or 18 years of age, such as 17 to 83 years of age, for example an adult of about 18 to about 80 years of age. In other specific examples of the aspects as defined herein, the subject is an adolescent. In still other specific examples of the aspects as defined herein, the subject is a child below about 17 years of age, or below about 18 years of age, such as a child of about 5 to about 17 years of age, such as about 5 to about 12 years of age, or a child of less than about 5 years of age, such as 1 month to 5 years of age, such as 1 to 6 months of age, or such as 6 to 12 months of age, or such as about 1 year to about 5 years of age, such as about 1 year, about 2 years, about 3 years, or about 4 years of age.

### Kits of Parts

In some examples, a kit of part for use in different aspects as defined herein, may be any manufacture (e.g., a package or container) comprising a number of parts, optionally comprising instructions for use in methods as described in aspects as defined herein. The manufacture is preferably promoted, distributed, or sold as a unit for performing the methods as defined herein.

In some examples of kits as defined herein, a kit is any manufacture (e.g., a package or container) comprising at least one reagent, for example, a probe for determining the genotype of a polymorphism, optionally with instructions for use in combination with a immunotherapy product as described herein.

In some examples, a kit as defined herein may further comprise means for collecting a biological sample, e.g. a blood sample.

In some examples of kits as defined herein, a kit comprises at least one allergen-specific immunotherapy product and further comprises instructions for use in combination with a method as defined herein.

In other examples of kits as defined herein, a kit is any manufacture (e.g., a package or container) comprising at least one immunotherapy product, and further comprising instructions for use in combination with detection of one or more SNPs of the 17q12-21 region, or any SNP in linkage disequilibrium with the one or more SNPs of the 17q12-21 region, e.g. instructions for use in methods as defined herein.

In preferred examples of kits as defined herein, a kit comprises at least one allergen-specific immunotherapy product and further comprises instructions for use in combination with detection of one or more SNPs of the 17q12-21 region, or any SNP in linkage disequilibrium with the one or more SNPs of the 17q12-21 region, e.g. instructions for use in combination with detection of one or more SNPs of Table 1 in methods as described herein, preferably detection of one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10).

In some examples of kits as defined herein, a kit of parts comprises
a) Means for quantifying the level of one or more Th2 markers selected from the group consisting of eosinophils, eosinophil cationic protein (ECP), tryptase and periostin; and
b) Instructions for use of said kit in combination with an immunotherapy product.

In some examples of kits as defined herein, a kit of parts comprises at least one allergen-specific immunotherapy product and further comprises instructions for use in combination with detection of one or more SNPs of the 17q12-21 region (e.g. one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10)), and use in combination with quantification of one or more Th2 markers selected from the group consisting of eosinophils, eosinophil cationic protein (ECP), tryptase and periostin.

In some examples, a kits of parts comprises
a) Means for detection of one or more SNPs of the 17q12-21 region (e.g. one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10)) and
b) Means for quantifying the level of one or more Th2 markers selected from the group consisting of eosinophils, eosinophil cationic protein (ECP), tryptase and periostin.

In some examples of kits as defined herein, a kit of parts may further comprise means for diagnosis of allergy or sensitization to an allergen, e.g. means for a skin prick test, or for measurement of IgE in a biological sample, e.g. a house dust mite skin prick test, or for detection of IgE specific to one or more house dust mite allergens.

In some examples of kits of parts as defined herein, a kit of parts may further comprise one or more second active ingredients, for separate, sequential or separate administration. Such second active ingredients may be selected from the group consisting of other immunotherapy products, corticosteroids (e.g. oral or inhaled) and other asthma treatment products such as SABA, LABA or LAMA.

### Other Aspects of the Invention

Another example of an aspect according to the invention provides the use of an allergen and/or antibody in the manufacture of a medicament for the treatment of atopic disease such as asthma, wherein said treatment comprises:
a) Detecting whether a biological sample obtained from said subject has one or more of:
   i. A single nucleotide polymorphisms (SNPs) selected from the group consisting of the single nucleic acid polymorphisms located in the chromosome 17q12-21 region and/or;
   ii. A SNP in linkage disequilibrium with one or more SNPs as defined in i) above;
b) Selecting said subject for treatment with said medicament and/or predicting response to treatment with said medicament if one or more SNPs of section a) are identified in biological sample from said subject.

Another example of an aspect according to the invention provides the use of an allergen and/or antibody in the manufacture of a medicament for the treatment of atopic disease such as asthma, wherein said treatment comprises:
a) Quantifying the level of one or more Th2 markers selected from the group consisting of eosinophils, eosinophil cationic protein (ECP), tryptase and periostin;
b) Selecting said subject for treatment with an immunotherapy product and/or predicting a beneficial response to treatment with an immunotherapy product in said subject, if the level of one or more markers of step a) is increased or above a reference level.

Still another example of an aspect of the invention provides for the use of one or more of a single nucleotide polymorphisms (SNPs) selected from the group consisting of:
i. A single nucleotide polymorphisms (SNPs) selected from the group consisting of the single nucleic acid polymorphisms located in the chromosome 17q12-21 region and/or;
ii. A SNP in linkage disequilibrium with one or more SNPs as defined in i) above;
   for predicting responsiveness of a subject to treatment with an immunotherapy product.

Still another example of an aspect of the invention provides for the use of one or more Th2 markers selected from the group consisting of eosinophils, eosinophil cationic protein (ECP), tryptase, and periostin for predicting responsiveness of a subject to treatment with an immunotherapy product.

Still another example of an aspect of the invention provides for the use of one or more of a single nucleotide polymorphisms (SNPs) selected from the group consisting of:
i. A single nucleotide polymorphisms (SNPs) selected from the group consisting of the single nucleic acid polymorphisms located in the chromosome 17q12-21 region and/or;
ii. A SNP in linkage disequilibrium with one or more SNPs as defined in i) above;
   for selecting a subject for treatment with an immunotherapy product.

Still another example of an aspect of the invention provides for the use of one or more Th2 markers selected from the group consisting of eosinophils, eosinophil cationic protein (ECP), tryptase, and periostin for selecting a subject for treatment with an immunotherapy product.

The following embodiments further define the aspects of the invention and examples thereof:

### Embodiments

Embodiment 1. A method for selecting a subject for treatment with an immunotherapy product for treatment of an atopic disease, e.g. asthma and/or predicting response to treatment with an immunotherapy product for treatment of an atopic disease, e.g. asthma comprising:
   a) Detecting whether a biological sample obtained from said subject has one or more of:
      i. A single nucleotide polymorphism (SNP) selected from the group consisting of the single nucleic acid polymorphisms located in the chromosome 17q12-21 region and/or;
      ii. A SNP in linkage disequilibrium with one or more SNPs as defined in i) above;
   b) Selecting said subject for treatment with an immunotherapy product and/or predicting a beneficial response to treatment with an immunotherapy product in said subject, if one or more SNPs of step a) are detected.
Embodiment 2. The method according to Embodiment 1, further comprising a treatment step c) wherein an immunotherapy product is administered to a subject that is either selected for treatment according to step b) and/or predicted to have a beneficial response to treatment according to step b).
Embodiment 3. The method according to any one of the preceding Embodiments, wherein the SNPs of ii. are located in the chromosome 17q12-21 region.
Embodiment 4. The method according to any one of the preceding Embodiments, wherein a SNP of i. is one or more SNPs selected from the group consisting of SNPs listed in Table 1, i.e. the group consisting of rs2941504 (SEQ ID NO: 5), rs2517955 (SEQ ID NO: 4), rs2952156 (SEQ ID NO: 6), rs907092 (SEQ ID NO: 1), rs9303277 (SEQ ID NO: 13), rs12936231 (SEQ ID NO: 17), rs8069176 (SEQ ID NO: 11), rs2305480 (SEQ ID NO: 3), rs11078927 (SEQ ID NO: 14), rs11078928 (SEQ ID NO: 15), rs2290400 (SEQ ID NO: 2), rs7216389 (SEQ ID NO: 10), rs4065275 (SEQ ID NO: 9), rs8076131 (SEQ ID NO: 12), rs12603332 (SEQ ID NO: 16), rs3894194 (SEQ ID NO: 8) and rs3859192 (SEQ ID NO: 7), or a complementary SNP thereof.
Embodiment 5. The method according to any one of the preceding Embodiments, wherein step a) is detecting whether the sample from said subject has one or more SNPs selected from the group consisting of SNPs listed in Table 1, i.e. the group consisting of rs2941504 (SEQ ID NO: 5), rs2517955 (SEQ ID NO: 4), rs2952156 (SEQ ID NO: 6), rs907092 (SEQ ID NO: 1), rs9303277 (SEQ ID NO: 13), rs12936231 (SEQ ID NO: 17), rs8069176 (SEQ ID NO: 11), rs2305480 (SEQ ID NO: 3), rs11078927 (SEQ ID NO: 14), rs11078928 (SEQ ID NO: 15), rs2290400 (SEQ ID NO: 2), rs7216389 (SEQ ID NO: 10), rs4065275 (SEQ ID NO: 9), rs8076131 (SEQ ID NO: 12), rs12603332 (SEQ ID NO: 16), rs3894194 (SEQ ID NO: 8) and rs3859192 (SEQ ID NO: 7), or a complementary SNP thereof.
Embodiment 6. The method according to any one of the preceding Embodiments, wherein step a) is detecting whether the sample from said subject has one or more SNPs selected from the group consisting of SNPs listed in Table 1 i.e. the group consisting of the rs2941504 (SEQ ID NO: 5) A allele, the rs2517955 (SEQ ID NO: 4) C allele, the rs2952156 (SEQ ID NO: 6) A allele, the rs907092 (SEQ ID NO: 1) G allele, the rs9303277 (SEQ ID NO: 13) C allele, the rs12936231 (SEQ ID NO: 17) C allele, the rs8069176 (SEQ ID NO: 11) G allele, the rs2305480 (SEQ ID NO: 3) G allele, the rs11078927 (SEQ ID NO: 14) C allele, the rs11078928 (SEQ ID NO: 15) T allele, the rs2290400 (SEQ ID NO: 2) T allele, the rs7216389 (SEQ ID NO: 10) T allele, the rs4065275 (SEQ ID NO: 9) G allele, the rs8076131 (SEQ ID NO: 12) A allele, the rs12603332 (SEQ ID NO: 16) C allele, the rs3894194 (SEQ ID NO: 8) A allele and the rs3859192 (SEQ ID NO: 7) T allele.
Embodiment 7. The method according to any one of the preceding Embodiments, wherein a SNP of i. is selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2), rs7216389 (SEQ ID NO: 10), rs3894194 (SEQ ID NO: 8) and rs3859192 (SEQ ID NO: 7) or a complementary SNP thereof.
Embodiment 8. The method according to any one of the preceding Embodiments, wherein step a) is detecting whether the sample from said subject has one or more SNPs selected from the group consisting of SNPs rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2), rs7216389 (SEQ ID NO: 10), rs3894194 (SEQ ID NO: 8) and rs3859192 (SEQ ID NO: 7) or a complementary SNP thereof.
Embodiment 9. The method according to any one of the preceding Embodiments wherein step a) is detecting whether the sample from said subject has one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10) or a complementary SNP thereof.
Embodiment 10. The method according to any one of the preceding Embodiments, wherein step a) is detecting whether the sample from said subject has one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10), or a complementary SNP thereof.
Embodiment 11. The method according to any one of the preceding Embodiments, wherein step a) is detecting whether the sample from said subject has the rs907092 (SEQ ID NO: 1) SNP, or a complementary SNP thereof.
Embodiment 12. The method according to any one of the preceding Embodiments, wherein step a) is detecting whether the sample from said subject has the rs9303277 (SEQ ID NO: 13) SNP, or a complementary SNP thereof.
Embodiment 13. The method according to any one of the preceding Embodiments, wherein step a) is detecting whether the sample from said subject has the rs8069176 (SEQ ID NO: 11) SNP, or a complementary SNP thereof.
Embodiment 14. The method according to any one of the preceding Embodiments, wherein step a) is detecting whether the sample from said subject has the rs2305480 (SEQ ID NO: 3) SNP, or a complementary SNP thereof.
Embodiment 15. The method according to any one of the preceding Embodiments, wherein step a) is detecting whether the sample from said subject has the rs11078927 (SEQ ID NO: 14) SNP, or a complementary SNP thereof.
Embodiment 16. The method according to any one of the preceding Embodiments, wherein step a) is detecting whether the sample from said subject has the rs2290400 (SEQ ID NO: 2) SNP, or a complementary SNP thereof.
Embodiment 17. The method according to any one of the preceding Embodiments, wherein step a) is detecting whether the sample from said subject has the rs7216389 (SEQ ID NO: 10) SNP, or a complementary SNP thereof.
Embodiment 18. The method according to any one of the preceding Embodiments, wherein said subject is selected for treatment with immunotherapy and/or a beneficial response to treatment using immunotherapy in said subject requires detection that said subject is a homozygote with respect to one or more SNPs of the chromosome 17q12-21 region.
Embodiment 19. The method according to any one of the preceding Embodiments, wherein said subject is selected for treatment with said immunotherapy product and/or predicted to have a beneficial response to treatment using said immunotherapy product if said subject is a homozygote with respect to one or more SNPs selected from the group consisting of rs2941504 (SEQ ID NO: 5), rs2517955 (SEQ ID NO: 4), rs2952156 (SEQ ID NO: 6), rs907092 (SEQ ID NO: 1), rs9303277 (SEQ ID NO: 13), rs12936231 (SEQ ID NO: 17), rs8069176 (SEQ ID NO: 11), rs2305480 (SEQ ID NO: 3), rs11078927 (SEQ ID NO: 14), rs11078928 (SEQ ID NO: 15), rs2290400 (SEQ ID NO: 2), rs7216389 (SEQ ID NO: 10), rs4065275 (SEQ ID NO: 9), rs8076131 (SEQ ID NO: 12), rs12603332 (SEQ ID NO: 16), rs3894194 (SEQ ID NO: 8) and rs3859192 (SEQ ID NO: 7).
Embodiment 20. The method according to any one of the preceding Embodiments, wherein said subject is selected for treatment with said immunotherapy product and/or predicted to have a beneficial response to treatment using said immunotherapy product if said subject is a homozygote with respect to one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2), rs7216389 (SEQ ID NO: 10), rs3894194 (SEQ ID NO: 8) and rs3859192 (SEQ ID NO: 7).
Embodiment 21. The method according to any one of the preceding Embodiments, wherein said subject is selected for treatment with said immunotherapy product and/or predicted to have a beneficial response to treatment using said immunotherapy product if said subject is a homozygote with respect to one or more SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10).
Embodiment 22. The method according to any one of the preceding Embodiments, wherein the immunotherapy product is selected from the group consisting of antibodies for treating asthma and allergen-specific immunotherapy products.
Embodiment 23. The method according to any one of the preceding Embodiments, wherein said immunotherapy product comprises one or more antibodies selected from the group consisting of anti-IgE (e.g. Omalizumab), anti-IL4Rα (e.g. Dupilumab), anti-IL4 (e.g VAK694), anti-IL13 (e.g. Tralokinumab and Lebrikizumab), anti-IL5 (e.g. Mepolizumab and Reslizumab), anti-IL5 receptor (e.g. Benralizumab), anti-IL25, anti-thymic stromal lymphopoietin (anti-TSLP) (e.g. Tezepelumab), anti-IL13/IL17 receptors (e.g. BITS7201A/RG7990) and anti-IL33 (e.g. AMG282).
Embodiment 24. The method according to any one of the preceding Embodiments, wherein said immunotherapy product is at least one product for allergen-specific immunotherapy, optionally administered in combination with one or more other immunotherapy products.
Embodiment 25. The method according to any one of the preceding Embodiments, wherein said immunotherapy product is an allergen-specific immunotherapy product.
Embodiment 26. The method according to any one of the preceding Embodiments, wherein said immunotherapy product is an allergen-specific immunotherapy product and comprises one or more immunogenic agents selected from the group consisting of a natural allergen extract, an allergoid extract, a recombinant wild-type allergen, a recombinant hypo-allergen, B cell and/or T cell epitope-containing fragments of an allergen, a plasmid DNA encoding the allergen or fragments thereof, and a mRNA encoding the allergen or fragments thereof.
Embodiment 27. The method according to any one of the preceding Embodiments, wherein said immunotherapy product is an allergen-specific immunotherapy product, and comprises of an allergen extract prepared from a natural source material and/or one or more recombinant allergens.
Embodiment 28. The method according to any one of the preceding Embodiments, wherein said immunotherapy product is for allergen-specific immunotherapy and comprises an allergen of a species selected from the group consisting of house dust mites (such as e.g. Dermatophagoides farinae, Dermatophagoides pterinussinus, and

Blomia tropicalis), grass pollen (such as e.g. Phleum pratense pollen, Dactylus glomerata pollen, Lolium perenne pollen, Poa pratensis pollen), tree pollen (such as e.g. birch pollen, hazel pollen, alder pollen, hornbeam pollen, beech pollen, oak pollen, cedar pollen, ash pollen, olive pollen), weed pollen (such as e.g. ragweed pollen, mug wort pollen), insect venom, animals (such as e.g. cockroach, cat, dog or horse), and food species (such as e.g. peanut, egg, cow's milk, fish, wheat, soy, peach, kiwi, hazelnut, shellfish (such as e.g. crustaceans, mollusks)), moulds (such as e.g. Aspergillus fumigatus) and fungi (such as e.g. Malassezia species).
Embodiment 29. The method according to any one of the preceding Embodiments, wherein said immunotherapy product is for allergen-specific immunotherapy and comprises allergen extract selected from the groups consisting of grass pollen extract, tree pollen allergen extract, weed pollen allergen extract, cedar pollen allergen extract, and house dust mite allergen extract.
Embodiment 30. The method according to any one of the preceding Embodiments, wherein said immunotherapy product is for allergen-specific immunotherapy and comprises a house dust mite allergen extract and/or a grass pollen extract.
Embodiment 31. The method according to any one of the preceding Embodiments, wherein said immunotherapy product is for allergen-specific immunotherapy and comprises a house dust mite allergen extract.
Embodiment 32. The method according to any one of the preceding Embodiments, wherein said immunotherapy product is for allergen-specific immunotherapy and comprises a grass pollen allergen extract.
Embodiment 33. The method according to any one of the preceding Embodiments, wherein said immunotherapy is used in combination with simultaneous, subsequent or singular administration of at least one second active ingredient useful for treatment of asthma.
Embodiment 34. The method according to any one of the preceding Embodiments, wherein said immunotherapy product is at least one product for allergen-specific immunotherapy, and is used in combination with simultaneous, subsequent or singular administration of at least one second active ingredient useful for treatment of asthma.
Embodiment 35. The method according to any one of the preceding Embodiments, wherein said immunoproduct is administered in combination with one or more secondary agents for asthma treatment selected from the group consisting of corticosteroids (inhaled, systemic and/or oral) e.g. fluticasone propionate, fluticasone furoate, budesonide, beclomethasone, β₂-adrenergic agonists (short acting or long acting), muscarinic receptor antagonists (e.g. long acting muscarinic receptor antagonists, LABA), anticholinergics, mast cell stabilizers, leukotriene antagonists or leukotriene modifiers, cromolyn sodium and methylxanthines.
Embodiment 36. The method according to any one of the preceding Embodiments, wherein said immunoproduct is administered in combination with inhaled corticosteroid (e.g. budesonide, fluticasone propionate, fluticasone furoate or beclomethasone) and optionally a short acting β₂-adrenergic agonist (e.g. salbutamol or terbutaline).
Embodiment 37. The method according to any one of the preceding Embodiments, wherein said immunoproduct is administered in combination with inhaled corticosteroid (e.g. budesonide) and optionally a short acting β₂-adrenergic agonist (e.g. salbutamol).
Embodiment 38. The method according to any one of the preceding Embodiments, wherein said immunoproduct is administered in combination with inhaled corticosteroid (e.g. budesonide, fluticasone propionate, fluticasone furoate or beclomethasone) and a long acting β₂-adrenergic agonist (e.g. salmeterol, formeterol, or vilanterol) and optionally a short acting β₂-adrenergic agonist (e.g. salbutamol or terbutaline).
Embodiment 39. The method according to any one of the preceding Embodiments, wherein said immunoproduct is administered in combination with inhaled corticosteroid (e.g. budesonide) and a long acting β₂-adrenergic agonist (e.g. salmeterol, formeterol, or vilanterol) and optionally a short acting β₂-adrenergic agonist (e.g. salbutamol).
Embodiment 40. The method according to any one of the preceding Embodiments, wherein said immunotherapy product is at least one product for allergen-specific immunotherapy, and is administered in combination with a corticosteroid (e.g. inhaled) and optionally a short acting β₂-adrenergic agonist.
Embodiment 41. The method according to any one of the preceding Embodiments, wherein said immunotherapy product is at least one product for allergen-specific immunotherapy, and is administered in combination with a inhaled corticosteroid (e.g. budesonide) and a long acting β₂-adrenergic agonist, and optionally a short acting β₂-adrenergic agonist (e.g. salbutamol).
Embodiment 42. The method according to any one of the preceding Embodiments, wherein said immunotherapy product is at least one product for allergen-specific immunotherapy, and is administered in combination with a inhaled corticosteroid (e.g. budesonide) and optionally a short acting β₂-adrenergic agonist (e.g. salbutamol).
Embodiment 43. The method according to any one of the preceding Embodiments, wherein said immunotherapy product is at least one product for house dust mite allergen-specific immunotherapy, and is administered in combination with a inhaled budesonide and optionally salbutamol.
Embodiment 44. The method according to any one of the preceding Embodiments, wherein said immunotherapy product is at least one product for allergen-specific immunotherapy, and is administered in combination with an antibody for treating asthma.
Embodiment 45. The method according to any one of the preceding Embodiments, wherein said immunotherapy product is at least one product for allergen-specific immunotherapy, and is administered in combination with an anti-IL4Rα antibody, e.g. Dupilumab.
Embodiment 46. The method according to any one of the preceding Embodiments, wherein said immunotherapy product is administered by parental administration, such as e.g. by subcutaneous, intradermal, intramuscular, intravenous or intrapulmonary injection.
Embodiment 47. The method according to any one of the preceding Embodiments, wherein said immunotherapy product is administered into the airways, such as by inhalation or nebulization.
Embodiment 48. The method according to any one of the preceding Embodiments, wherein said immunotherapy product is administered by oromucosal administration, such as buccal and/or sublingual administration.
Embodiment 49. The method according to any one of the preceding Embodiments, wherein said immunotherapy product is administered by sublingual administration.
Embodiment 50. The method according to any one of the preceding Embodiments, wherein said immunotherapy product is for sublingual allergen-specific immunotherapy or sub-cutaneous allergen-specific immunotherapy.
Embodiment 51. The method according to any one of the preceding Embodiments, wherein said immunotherapy product is for oromucosal administration of a house dust mite allergen extract to a subject in need thereof, such as sublingual administration of a house dust mite allergen extract to a subject in need thereof.
Embodiment 52. The method according to any one of the preceding Embodiments, wherein said immunotherapy product is sublingual administration of a house dust mite allergen extract to a subject in need thereof, and is administered in combination with a inhaled corticosteroid (budesonide) and optionally a β₂-adrenergic agonist (salbutamol).
Embodiment 53. The method according to any one of the preceding Embodiments, wherein said immunotherapy product is for allergen-specific immunotherapy by oromucosal administration of a grass pollen allergen extract to said subject, such as sublingual administration of a grass pollen allergen extract to said subject.
Embodiment 54. The method according to any one of the preceding Embodiments, wherein said immunotherapy product is for allergen-specific immunotherapy by sublingual administration of a tablet comprising an allergen extract to said subject.
Embodiment 55. The method according to any one of the preceding Embodiments, wherein said immunotherapy product is for allergen-specific immunotherapy by sublingual administration of a fast dispersing tablet comprising an allergen extract to said subject.
Embodiment 56. The method according to any one of the preceding Embodiments, wherein said immunotherapy product is for allergen-specific immunotherapy by sublingual administration of a liquid formulation comprising an allergy extract to said subject.
Embodiment 57. The method according to any one of the preceding Embodiments, wherein said subject is of European, Hispanic (latino) and/or Asian descent.
Embodiment 58. The method according to any one of the preceding Embodiments, wherein said subject is not of African or Afro American descent.
Embodiment 59. The method according to any one of the preceding Embodiments, wherein said subject is of European ancestry or descent.
Embodiment 60. The method according to any one of the preceding Embodiments, wherein said subject is an adult.
Embodiment 61. The method according to any one of the preceding Embodiments wherein said subject is an adult above 17 or 18 years of age, such as 17 to 83 years of age.
Embodiment 62. The method according to any one of the preceding Embodiments wherein said subject is an adult of about 18 to about 80 years of age.
Embodiment 63. The method according to any one of the preceding Embodiments, wherein said subject is a child.
Embodiment 64. The method according to any one of the preceding Embodiments, wherein said subject is a child below about 17 or below about 18 years of age.
Embodiment 65. The method according to any one of the preceding Embodiments, wherein said subject is a child of about 5 to about 18 years of age, such as about 5 to about 12 years of age.
Embodiment 66. The method according to any one of the preceding Embodiments, wherein said subject is a child of less than about 5 years of age, such as 1 month to 5 years of age, such as 1 to 6 months of age, or such as 6 to 12 months of age, or such as about 1 year to about 5 years of age, such as about 1 year, about 2 years, about 3 years, or about 4 years of age.
Embodiment 67. The method according to any one of the preceding Embodiments, wherein said subject has been diagnosed with and/or has experienced symptoms and signs of one or more atopic diseases.
Embodiment 68. The method according to any one of the preceding Embodiments, wherein said subject has been diagnosed with and/or experienced symptoms and signs of one or more atopic diseases selected from the group consisting of atopic dermatitis, asthma, allergic rhinitis, allergic conjunctivitis and allergic rhinoconjunctivitis.
Embodiment 69. The method according to any one of the preceding Embodiments, wherein said subject has been diagnosed with and/or experienced symptoms and signs of asthma.
Embodiment 70. The method according to any one of the preceding Embodiments, wherein said subject has previously been diagnosed with and/or experienced symptoms of allergy against or sensitivity to one or more allergens.
Embodiment 71. The method according to any one of the preceding Embodiments, wherein said subject has been diagnosed with allergy by measurement of IgE to one or more allergens, and/or by a positive skin prick test to one or more allergens.
Embodiment 72. The method according to any one of the preceding Embodiments, wherein said subject has previously been diagnosed with and/or experienced symptoms or signs of early on-set asthma, and/or allergic asthma.
Embodiment 73. The method according to any one of the preceding Embodiments, wherein said subject has previously been diagnosed with and/or experienced symptoms of one or more degrees of severity of asthma selected from the group consisting of mild asthma, moderate asthma, severe asthma and refractory asthma.
Embodiment 74. The method according to any one of the preceding Embodiments, wherein said subject has not previously been diagnosed with asthma or experienced one or more symptoms of asthma.
Embodiment 75. The method according to any one of the preceding Embodiments, wherein said subject has not previously been diagnosed with asthma, or has not experienced one or more symptoms and signs of asthma, but has one or more other atopic diseases, e.g. allergic rhinitis, allergic conjunctivitis, or allergic rhinoconjunctivis.
Embodiment 76. The method according to any one of the preceding Embodiments, wherein said subject has, or has previously been diagnosed with and/or experienced symptoms of one or more degrees of severity of asthma selected from the group consisting of intermittent asthma, mild asthma, moderate asthma, severe asthma and refractory asthma.
Embodiment 77. The method according to any one of the preceding Embodiments, wherein said subject has previously been diagnosed with and/or experienced symptoms of intermittent and/or mild asthma.
Embodiment 78. The method according to any one of the preceding Embodiments, wherein said subject has, or has previously been diagnosed with and/or experienced symptoms of one or more degrees of severity of asthma selected from the group consisting of moderate asthma, severe asthma and refractory asthma.
Embodiment 79. The method according to any one of the preceding Embodiments, wherein said subject has, has previously been diagnosed with and/or experienced signs and symptoms of moderate asthma and/or severe asthma, such as moderate asthma, or such as severe asthma.
Embodiment 80. The method according to any one of the preceding Embodiments, wherein said subject has, previously been diagnosed with and/or experienced signs and symptoms of moderate allergic asthma and/or severe allergic asthma, such as moderate allergic asthma, or such as severe allergic asthma.
Embodiment 81. The method according to any one of the preceding Embodiments, wherein said subject has, previously been diagnosed with and/or experienced signs and symptoms of a moderate and/or severe asthma exacerbation, such as a moderate asthma exacerbation, or such as a severe asthma exacerbation.
Embodiment 82. The method according to any one of the preceding Embodiments, wherein symptoms and/or signs of asthma are selected from the group consisting of wheezing (e.g. a whistling, squeaky sound during breathing), shortness of breath, chest tightness, rapid breathing, increased coughing, fatigue, nocturnal awakening and/or problems sleeping (for example due to coughing or difficulty breathing), decreased lung function, such as decreased peak expiratory flow (PEF), decreases forced expiratory volume in one second (FEV1), or such as decreased forced vital capacity (FVC), or decreased ratio of FEV1 compared to FVC (FEV1/FVC), increased fraction of exhaled nitric oxide (FeNO), and increased number of eosinophils in blood, and/or sputum.
Embodiment 83. The method according to any one of the preceding Embodiments, wherein said subject has previously been diagnosed with house dust mite allergy or sensitivity to house dust mite, and the immunotherapy product comprises a house dust mite allergen (such as a house dust mite allergen extract) and/or an antibody for treating asthma.
Embodiment 84. The method according to any one of the preceding Embodiments, wherein said subject has previously been diagnosed with house dust mite allergy, such as house dust mite-related allergic rhinitis or house dust mite-related allergic conjunctivitis, and the immunotherapy product comprises a house dust mite allergen, such as a house dust mite allergen extract.
Embodiment 85. The method according to any one of the preceding Embodiments, wherein said subject has previously been diagnosed with grass pollen allergy or sensitivity to grass pollen, and the immunotherapy product comprises a grass pollen allergen (such as a grass pollen allergen extract) and/or an antibody for treatment of asthma.
Embodiment 86. The method according to any one of the preceding Embodiments, wherein said subject has previously been diagnosed with grass pollen allergy, such as grass pollen-related allergic rhinitis or grass pollen-related allergic conjunctivitis, and the immunotherapy product comprises a grass pollen allergen (such as a grass pollen allergen extract).
Embodiment 87. The method according to any one of the preceding Embodiments, wherein one or more close relatives of said subject has been diagnosed with and/or experienced symptoms of atopic disease (such as allergic rhinitis, allergic conjunctivitis and/or asthma).
Embodiment 88. The method according to any one of the preceding Embodiments, wherein one or more parent(s) of said subject has been diagnosed with and/or experienced symptoms of atopic disease (such as allergic rhinitis, allergic conjunctivitis and/or asthma).
Embodiment 89. The method according to any one of the preceding Embodiments, wherein one or more parent(s) of said subject has been diagnosed with and/or experienced symptoms of moderate to severe atopic disease, such as moderate to severe allergic rhinitis or allergic conjunctivitis and/or moderate to severe asthma.
Embodiment 90. The method according to any one of the preceding Embodiments, wherein said response to treatment is selected from the group consisting of preventing, reducing risk of, treatment, ameliorating symptoms and signs of one or more atopic diseases selected from the group consisting of atopic dermatitis, allergy and asthma.
Embodiment 91. The method according to any one of the preceding Embodiments, wherein said response to treatment is selected from the group consisting of preventing, reducing risk of, treatment, ameliorating symptoms and signs of at least asthma.
Embodiment 92. The method according to any one of the preceding Embodiments, wherein said response to treatment is a reduced risk of asthma exacerbations and/or deterioration in asthma symptoms.
Embodiment 93. The method according to any one of the preceding Embodiments, wherein the signs and symptoms of asthma are selected from the group consisting of wheezing (e.g. a whistling, squeaky sound during breathing), shortness of breath, chest tightness, rapid breathing, increased coughing, fatigue, nocturnal awakening and/or problems sleeping (for example due to coughing or difficulty breathing), decreased lung function, such as decreased peak expiratory flow (PEF), decreases forced expiratory volume in one second (FEV1), or such as decreased forced vital capacity (FVC), or decreased ratio of FEV1 compared to FVC (FEV1/FVC), increased fraction of exhaled nitric oxide (FeNO), and increased number of eosinophils in blood and/or sputum.
Embodiment 94. The method according to any one of the preceding Embodiments, wherein said response to treatment reduces risk, rate or frequency, of moderate to severe asthma exacerbations.
Embodiment 95. The method according to any one of the preceding Embodiments, wherein said response to treatment is a ameliorating of one or more signs and symptoms of asthma.
Embodiment 96. The method according to any one of the preceding Embodiments, wherein said response to treatment is a reduced risk of experiencing one or more asthma signs and/or symptoms and/or need for use of asthma medication.
Embodiment 97. The method according to any one of the preceding Embodiments, wherein said response to treatment is a reduced use of asthma medication.
Embodiment 98. The method according to any one of the preceding Embodiments, wherein said response to treatment is a reduced need for use of asthma medication selected from the group consisting of inhaled, systemic or oral corticosteroids.
Embodiment 99. The method according to any one of the preceding Embodiments, wherein said response to treatment is a reduced need for use of systemically administered corticosteroids.
Embodiment 100. The method according to any one of the preceding Embodiments, wherein said response to treatment is a reduced need for use of β₂-adrenergic agonists.
Embodiment 101. The method according to any one of the preceding Embodiments, wherein said response to treatment is a reduced use of inhaled corticosteroid in combination with a β₂-adrenergic agonist.
Embodiment 102. A method of treating, preventing, delaying onset, or reducing risk of an atopic disease, such as asthma, in a subject in need thereof, said method comprising:
   a) Obtaining and/or providing a biological sample comprising nucleic acids from said subject; and
   b) Detecting whether the nucleic acids obtained from said subject has one or more of:
      i. A single nucleotide polymorphisms (SNPs) selected from the group consisting of the single nucleic acid polymorphisms located in the chromosome 17q12-21 region and/or;
      ii. A SNP in linkage disequilibrium with one or more SNPs as defined in i) above;
   c) Identifying or selecting said subject for treatment with immunotherapy if one or more SNPs of step b) are identified in the nucleic acids comprised in said biological sample; and
   d) Administering an immunotherapy product to said subject identified or selected according to step c).
Embodiment 103. The immunotherapy product according to Embodiment 102, wherein said SNP is further defined by any one of the preceding Embodiments 3 to 21.
Embodiment 104. The method according to any one of Embodiments 102 to 103, wherein the immunotherapy product is selected from the group consisting of antibodies and allergen-specific immunotherapy.
Embodiment 105. The method according to any one of Embodiments 102 to 104, wherein the immunotherapy product is defined as in any one of Embodiments 22 to 56.
Embodiment 106. The method according to any one of Embodiments 102 to 105, wherein the immunotherapy product is an allergen-specific immunotherapy product.
Embodiment 107. The method according to any one of Embodiments 102 to 106, wherein said subject is defined by any one of Embodiments 57 to 89.
Embodiment 108. The method according to any one of Embodiments 102 to 107, wherein said atopic disease is defined by any one of Embodiments 67 to 89.
Embodiment 109. The method according to any one of Embodiments 102 to 108, wherein the method is performed to obtain a response to treatment as defined by any one of Embodiments 90 to 101.
Embodiment 110. The method according to any one of Embodiments 102 to 109, wherein said method reduces the risk of asthma exacerbations and/or deterioration in symptoms and signs of asthma.
Embodiment 111. The method according to any one of Embodiments 102 to 110, wherein said method reduces the risk of moderate to severe asthma exacerbations.
Embodiment 112. The method according to any one of Embodiments 102 to 111, wherein said method reduces risk of experiencing one or more signs and symptoms of asthma and/or use of asthma medication.
Embodiment 113. The method according to any one of Embodiments 102 to 112, wherein said method reduces risk of experiencing, and/or ameliorates one or more signs and symptoms of asthma.
Embodiment 114. The method according to any one of Embodiments 102 to 113, wherein said method reduces use of asthma medication (e.g. inhaled, systemic or oral corticosteroid) and/or β₂-adrenergic agonists.
Embodiment 115. The method according to Embodiments 102 to 114, wherein said method further comprises administration of at least one second active ingredient useful for treatment of asthma.
Embodiment 116. The method according to Embodiments 102 to 115, wherein said method further comprises administration of an antibody useful for treatment of asthma.
Embodiment 117. The method according to Embodiments 102 to 116, wherein said method further comprises administration of one or more second agents for asthma treatment selected from the group consisting of corticosteroids (inhaled, systemic and/or oral), β₂-adrenergic agonists (short acting or long acting), muscarinic receptor antagonists (e.g. long acting muscarinic receptor antagonists, LAMA), anticholinergics, mast cell stabilizers, leukotriene antagonists or leukotriene modifiers, cromolyn sodium and methylxanthines.
Embodiment 118. The method according to Embodiments 102 to 117, wherein said method further comprises administration of inhaled corticosteroid (e.g. budesonide, fluticasone propionate, fluticasone furoate or beclomethasone) and optionally a short acting β₂-adrenergic agonist (e.g. salbutamol or terbutaline).
Embodiment 119. The method according to Embodiments 102 to 118, wherein said method further comprises administration of inhaled corticosteroid (e.g. budesonide) and optionally a short acting β₂-adrenergic agonist (e.g. salbutamol).
Embodiment 120. The method according to Embodiments 102 to 119, wherein said method further comprises administration of inhaled corticosteroid (e.g. budesonide, fluticasone propionate, fluticasone furoate or beclomethasone) and a long acting β₂-adrenergic agonist (e.g. salmeterol, formeterol, or vilanterol) and optionally a short acting β₂-adrenergic agonist (e.g. salbutamol or terbutaline).
Embodiment 121. The method according to Embodiments 102 to 120, wherein said method further comprises administration of inhaled corticosteroid (e.g. budesonide) and a long acting β₂-adrenergic agonist (e.g. salmeterol, formeterol, or vilanterol) and optionally a short acting β₂-adrenergic agonist (e.g. salbutamol).
Embodiment 122. An immunotherapy product for use in a method of treating, preventing or reducing risk of one or more atopic diseases, such as asthma, in a selected subject, said method comprising:
   a) Detecting whether a biological sample obtained from said subject from said subject has one or more of:
      i. A single nucleotide polymorphisms (SNPs) selected from the group consisting of the single nucleic acid polymorphisms located in the chromosome 17q12-21 region and/or;
      ii. A SNP in linkage disequilibrium with one or more SNPs as defined in i) above;
   b) Selecting and treating said subject with said immunotherapy product if one or more SNPs of step a) are identified in the biological sample from said subject.
Embodiment 123. The immunotherapy product according to Embodiment 122, wherein said SNP is further defined by any one of the preceding Embodiments 3 to 21.
Embodiment 124. The immunotherapy product according to any one of Embodiments 122 and 123, wherein said immunotherapy product is further defined by any one of the preceding Embodiments 22 to 56.
Embodiment 125. The immunotherapy product according to any one of the preceding Embodiments 122 to 124, wherein said immunotherapy product is for allergen-specific immunotherapy.
Embodiment 126. The immunotherapy product according to any one of Embodiments 122 to 125, wherein said subject is further defined by any one of Embodiments 57 to 89.
Embodiment 127. The immunotherapy product according to any one of Embodiments 122 to 126, wherein said immunotherapy product is for allergen-specific immunotherapy, and comprises a house dust mite allergen extract for sublingual administration.
Embodiment 128. The immunotherapy product according to any one of Embodiments 122 to 127, wherein said immunotherapy product is for allergen-specific immunotherapy, and comprises a house dust mite allergen extract formulated as a fast dispersing tablet for sublingual administration.
Embodiment 129. The immunotherapy product according to any one of Embodiments 122 to 128, wherein said immunotherapy product is for allergen-specific immunotherapy, and comprises a grass pollen allergen extract for sublingual administration.
Embodiment 130. The immunotherapy product according to any one of Embodiments 122 to 129, wherein said immunotherapy product is for allergen-specific immunotherapy, and comprises a grass pollen allergen extract formulated as a fast dispersing tablet for sublingual administration.
Embodiment 131. The immunotherapy product according to any one of Embodiments 122 to 130, wherein said method is to obtain a response as defined according to any one of Embodiments 90 to 101.
Embodiment 132. The immunotherapy product according to any one of Embodiments 122 to 131, wherein said method further comprises simultaneous, subsequent or singular administration of at least one second active ingredient useful for treatment of asthma.
Embodiment 133. The immunotherapy product according to any one of the preceding Embodiments 122 to 132, wherein said method further comprises administration of one or more secondary agents for asthma treatment selected from the group consisting of corticosteroids (inhaled, systemic and/or oral) e.g. fluticasone propionate, fluticasone furoate, budesonide, beclomethasone, β₂-adrenergic agonists (short acting or long acting), muscarinic receptor antagonists (e.g. long acting muscarinic receptor antagonists), anticholinergics, mast cell stabilizers, leukotriene antagonists or leukotriene modifiers, cromolyn sodium and methylxanthines.
Embodiment 134. The immunotherapy product according to any one of Embodiments 122 to 133, wherein said method further comprises administration of inhaled corticosteroid (e.g. budesonide, fluticasone propionate, fluticasone furoate or beclomethasone) and optionally a short acting β₂-adrenergic agonist (e.g. salbutamol or terbutaline).
Embodiment 135. The immunotherapy product according to any one of Embodiments 122 to 134, wherein said method further comprises administration of inhaled corticosteroid (e.g. budesonide) and optionally a short acting β₂-adrenergic agonist (e.g. salbutamol).
Embodiment 136. The immunotherapy product according to any one of Embodiments 122 to 135, wherein said method further comprises administration of inhaled corticosteroid (e.g. budesonide, fluticasone propionate, fluticasone furoate or beclomethasone) and a long acting β₂-adrenergic agonist (e.g. salmeterol, formeterol, or vilanterol) and optionally a short acting β₂-adrenergic agonist (e.g. salbutamol or terbutaline).
Embodiment 137. The immunotherapy product according to any one of Embodiments 122 to 136, wherein said method further comprises administration of inhaled corticosteroid (e.g. budesonide) and a long acting β₂-adrenergic agonist (e.g. salmeterol, formeterol, or vilanterol) and optionally a short acting β₂-adrenergic agonist (e.g. salbutamol).
Embodiment 138. The immunotherapy product according to any one of Embodiments 122 to 137, for reducing risk of asthma exacerbations and/or reduce risk of deterioration of one or more symptoms and signs of asthma.
Embodiment 139. The immunotherapy product according to any one of the Embodiments 122 to 138, for reducing risk of moderate to severe asthma exacerbations.
Embodiment 140. Use of an allergen and/or antibody in the manufacture of a medicament for the treatment of atopic disease such as asthma, wherein said treatment comprises:
   a) Detecting whether a biological sample obtained from said subject has one or more of:
      i. A single nucleotide polymorphisms (SNPs) selected from the group consisting of the single nucleic acid polymorphisms located in the chromosome 17q12-21 region and/or;
      ii. A SNP in linkage disequilibrium with one or more SNPs as defined in i) above;
   b) Selecting said subject for treatment with said medicament and/or predicting response to treatment with said medicament if one or more SNPs of section b) are identified in biological sample from said subject.
Embodiment 141. The use according to Embodiment 140, wherein said medicament is a immunotherapy product.
Embodiment 142. The use according to any one of Embodiments 140 and 141, wherein said medicament is a immunotherapy product defined according to any one of the Embodiments 22 to 56 and 122 to 139.
Embodiment 143. A kit of parts comprising:
   c) Means for detection of one or more SNPs selected from the groups i) and/or ii):
      i. A single nucleotide polymorphism (SNP) selected from the group consisting of the single nucleic acid polymorphisms located in the chromosome 17q12-21 region and/or;
      ii. A SNP in linkage disequilibrium with one or more SNPs as defined in i) above;
   d) Instructions for use of said kit in combination with an immunotherapy product.
Embodiment 144. The kit of parts according to Embodiment 143, said method detecting a SNP as defined according to any one of the Embodiments 3 to 21.
Embodiment 145. The kit of parts according to any one of Embodiments 143 to 144, wherein the immunotherapy product is defined according to any one of Embodiments 22 to 56 and 122 to 139.
Embodiment 146. The kit of parts according to any one of Embodiments 143 to 145, further comprising means for obtaining one or more biological samples.
Embodiment 147. The kit of parts according to any one of Embodiments 143 to 146, further comprising means for diagnosis of allergy or sensitization to an allergen.
Embodiment 148. The kit of parts according to any one of Embodiments 143 to 147, further comprising means for a skin prick test or for measurement of IgE in a biological sample.
Embodiment 149. A kit of parts comprising:
   a) An immunotherapy product, and
   b) Instructions for use of said immunotherapy product in combination with detection of:
      i. A single nucleotide polymorphism (SNP) selected from the group consisting of the single nucleic acid polymorphisms located in the chromosome 17q12-21 region and/or;
      ii. A SNP in linkage disequilibrium with one or more SNPs as defined in i) above;
Embodiment 150. The kit of parts according to Embodiment 149, said method detecting a SNP as defined according to any one of Embodiments 3 to 21.
Embodiment 151. The kit of parts according to any one of Embodiments 149 to 150, wherein the immunotherapy product is defined according to any one of Embodiments 22 to 56 and 122 to 139.
Embodiment 152. The kit of parts according to any one of Embodiments 149 to 151, further comprising means for detection of one or more SNPs as defined in Embodiments 3 to 21.
Embodiment 153. The kit of parts according to any one of Embodiments 149 to 152, further comprising means for obtaining one or more biological samples.
Embodiment 154. Use of one or more of a single nucleotide polymorphisms (SNPs) selected from the group consisting of:
   a) A single nucleotide polymorphisms (SNPs) selected from the group consisting of the single nucleic acid polymorphisms located in the chromosome 17q12-21 region and/or;
   b) A SNP in linkage disequilibrium with one or more SNPs as defined in i) above;
2. for predicting responsiveness of a subject to treatment with an immunotherapy product.
Embodiment 155. The use according to Embodiment 154, wherein the immunotherapy product is defined by any one of the Embodiments 22 to 56 and 122 to 139.
Embodiment 156. Use of one or more of a single nucleotide polymorphisms (SNPs) selected from the group consisting of:
   a) A single nucleotide polymorphisms (SNPs) selected from the group consisting of the single nucleic acid polymorphisms located in the chromosome 17q12-21 region and/or;
   b) A SNP in linkage disequilibrium with one or more SNPs as defined in i) above;
      for selecting a subject for treatment with an immunotherapy product.
Embodiment 157. The use according to Embodiment 156, wherein the immunotherapy product is defined by any one of Embodiments 22 to 56 and 122 to 139.
Embodiment 158. A method for selecting a subject for treatment with an immunotherapy product for treatment of an atopic disease, e.g. asthma and/or predicting response to treatment with an immunotherapy product for treatment of an atopic disease, e.g. asthma comprising:
   a) Quantifying the level of one or more Th2 markers selected from the group consisting of eosinophils, eosinophil cationic protein (ECP), tryptase, periostin, and total IgE;
   b) Selecting said subject for treatment with an immunotherapy product and/or predicting a beneficial response to treatment with an immunotherapy product in said subject, if the level of one or more markers of step a) is increased or above a reference level.
Embodiment 159. The method according to Embodiment 158, further comprising a treatment step c) wherein an immunotherapy product is administered to a subject that is either selected for treatment according to step b) and/or predicted to have a beneficial response to treatment according to step b).
Embodiment 160. The method according to any one of Embodiments 158 to 159, wherein the reference level is predetermined in a group of subjects diagnosed with the same atopic disease as said subject.
Embodiment 161. The method according to any one of Embodiments 158 to 160, wherein the reference level is predetermined in a group of subjects diagnosed with one or more atopic diseases.
Embodiment 162. The method according to any one of Embodiments 158 to 161, wherein the reference level is predetermined in a group of subjects diagnosed with one or more diseases selected from the group consisting of allergic rhinitis, allergic conjunctivitis and allergic asthma.
Embodiment 163. The method according to any one of Embodiments 158 to 162, wherein the reference level is predetermined in a group of subjects diagnosed with allergic asthma.
Embodiment 164. The method according to any one of Embodiments 158 to 163, wherein the reference level is predetermined in a group of subjects diagnosed with moderate or severe allergic asthma.
Embodiment 165. The method according to any one of Embodiments 158 to 164, wherein the reference level is predetermined in a group of healthy subjects.
Embodiment 166. The method according to any one of Embodiments 158 to 165, wherein the reference level is predetermined in a group of randomly selected subjects.
Embodiment 167. The method according to any one of Embodiments 158 to 166, wherein the reference level is the mean or median value in a group of subjects.
Embodiment 168. The method according to any one of Embodiments 158 to 167, wherein the level of one or more Th2 markers selected from the group consisting of eosinophil count, eosinophil cationic protein (ECP), tryptase, and periostin is quantified;
Embodiment 169. The method according to any one of Embodiments 158 to 168, wherein at least the level of eosinophils is quantified.
Embodiment 170. The method according to any one of Embodiments 158 to 169, wherein at least the level of blood eosinophil count is quantified.
Embodiment 171. The method according to any one of Embodiments 158 to 170, wherein at least the level of eosinophil cationic protein (ECP) is quantified.
Embodiment 172. The method according to any one of Embodiments 158 to 171, wherein at least the level of serum eosinophil cationic protein (ECP) is quantified.
Embodiment 173. The method according to any one of Embodiments 158 to 172, wherein at least the level of tryptase is quantified.
Embodiment 174. The method according to any one of Embodiments 158 to 173, wherein at least the level of serum tryptase is quantified.
Embodiment 175. The method according to any one of Embodiments 158 to 174, wherein at least the level of periostin is quantified.
Embodiment 176. The method according to any one of Embodiments 158 to 175, wherein at least the level of serum periostin is quantified.
Embodiment 177. The method according to any one of Embodiments 158 to 176, wherein at least the level of total IgE is quantified.
Embodiment 178. The method according to any one of Embodiments 158 to 177, wherein at least the level of total IgE is quantified.
Embodiment 179. The method according to any one of Embodiments 158 to 178, wherein the levels of eosinophil count and eosinophil cationic protein (ECP) is quantified.
Embodiment 180. The method according to any one of Embodiments 158 to 179, wherein the levels of eosinophil count and tryptase is quantified.
Embodiment 181. The method according to any one of Embodiments 158 to 180, wherein the levels of eosinophil count and periostin is quantified.
Embodiment 182. The method according to any one of Embodiments 158 to 181, wherein the levels of eosinophil cationic protein (ECP) and tryptase is quantified.
Embodiment 183. The method according to any one of Embodiments 158 to 182, wherein the levels of eosinophil cationic protein (ECP) and periostin is quantified.
Embodiment 184. The method according to any one of Embodiments 158 to 183, wherein the levels of tryptase and periostin is quantified.
Embodiment 185. The method according to any one of Embodiments 158 to 184, wherein the levels of eosinophil count, eosinophil cationic protein (ECP) and tryptase is quantified.
Embodiment 186. The method according to any one of Embodiments 158 to 185, wherein the levels of eosinophil count, eosinophil cationic protein (ECP) and periostin is quantified.
Embodiment 187. The method according to any one of Embodiments 158 to 186, wherein the levels of eosinophil count, tryptase, and periostin is quantified.
Embodiment 188. The method according to any one of Embodiments 158 to 187, wherein the levels of eosinophil cationic protein (ECP), tryptase and periostin is quantified.
Embodiment 189. The method according to any one of Embodiments 158 to 188, wherein the level of eosinophil count is quantified in blood, e.g. whole blood optionally treated with anti-coagulant.
Embodiment 190. The method according to any one of Embodiments 158 to 189, wherein the level of eosinophil cationic protein (ECP), tryptase, periostin and total IgE is quantified in a blood serum.
Embodiment 191. The method according to any one of Embodiments 158 to 190, wherein the levels of one or more of the biomarkers selected from the group consisting of blood eosinophil count, serum eosinophil cationic protein (ECP), serum tryptase and serum periostin is quantified.
Embodiment 192. The method according to any one of Embodiments 158 to 191, wherein the reference level for blood eosinophil count is 150 cells/microliter or higher, such as above 200 cells/microliter, such as from 200 cells/microliter to 500 cells/microliter, such as 225 cells/microliter, such as 250 cells/microliter, such as 275 cells/microliter, or such as from 200 to 300 cells/microliter, such as 225 cells/microliter, such as 250 cells/microliter, or such as 275 cells/microliter, or such as from 300 to 400 cells/microliter, such as 325 cells/microliter, such as 350 cells/microliter, or such as 375 cells/microliter, such as from 400 to 500 cells/microliter, such as 425 cells/microliter, such as 450 cells/microliter, or such as 475 cells/microliter, or such as 600 to 800 cells/microliter or more.
Embodiment 193. The method according to any one of Embodiments 158 to 192, wherein the reference level for blood eosinophil count is about 200 cells/microliter.
Embodiment 194. The method according to any one of Embodiments 158 to 193, wherein the reference level for blood eosinophil count is about 400 cells/microliter.
Embodiment 195. The method according to any one of Embodiments 158 to 194, wherein the reference level for serum ECP is 3 micrograms/L or higher, such as from 3 to 8 micrograms/L, such as 4 micrograms/L, 5 micrograms/L, 6 micrograms/L or 7 micrograms/L, or such as from 8 to 14 micrograms/L, such as 8 micrograms/L, 9 micrograms/L, 10 micrograms/L, 11 micrograms/L, 12 micrograms/L, 13 micrograms/L, or from 14 micrograms/L or higher, such as from 14 to 20 micrograms/L, such as 14 to 16 micrograms/L, such as 15 micrograms/L, or such as from 16 to 18 micrograms/L, such as 17 micrograms/L, or such as from 18 to 20 micrograms/L, such as 19 micrograms/L, or such as from 20 to 40 micrograms/L, such as from 20 to 30 micrograms/L, such as 20 to 22 micrograms/L, such as 21 micrograms/L, or such as from 22 to 24 micrograms/L, such as 23 micrograms/L, or such as from 24 to 26 micrograms/L, such as 25 micrograms/L, or such as from 26 to 28 micrograms/L, such as 27 micrograms/L, or such as from 28 to 30 micrograms/L, e.g. 29 micrograms/L, or such as from 30 to 40 micrograms/L, such as 30 to 32 micrograms/L, such as 33 micrograms/L, or such as from 32 to 34 micrograms/L, such as 33 micrograms/L, or such as from 34 to 36 micrograms/L, such as 35 micrograms/L, or such as from 36 to 38 micrograms/L, such as 37 micrograms/L, or such as from 38 to 40 micrograms/L, such as 39 micrograms/L, or such as above 40 micrograms/L, such as above 50 micrograms/L.
Embodiment 196. The method according to any one of Embodiments 158 to 195, wherein the reference level for serum ECP is about 14 micrograms/L, such as 14. 4 micrograms/L.
Embodiment 197. The method according to any one of Embodiments 158 to 196, wherein the reference level for serum ECP is about 29 micrograms/L such as 29. 4 micrograms/L.
Embodiment 198. The method according to any one of Embodiments 158 to 197, wherein the reference level for serum periostin is above 15 nanograms/mL, such as from 15 to 40 nanograms/mL, such as from 15 to 17 nanograms/mL, or such as from 17 to 20 nanograms/mL, or such as from 20 to 35 nanograms/mL, such as 20 nanograms/mL, or such as 21 nanograms/mL, such as 22 nanograms/mL, such as 23 nanograms/mL, such as 24 nanograms/mL, such as 25 nanograms/mL, such as 26 nanograms/mL, such as 27 nanograms/mL, such as 28 nanograms/mL, such as 29 nanograms/mL, such as 30 nanograms/mL, such as 31 nanograms/mL, such as 32 nanograms/mL, such as 33 nanograms/mL, such as 34 nanograms/mL, or such as from 35 to 40 nanograms/mL, or such as above 40 nanograms/mL.
Embodiment 199. The method according to any one of Embodiments 158 to 198, wherein the reference level for serum periostin is about 21 nanograms/mL.
Embodiment 200. The method according to any one of Embodiments 158 to 199, wherein the reference level for serum periostin is about 27 nanograms/mL.
Embodiment 201. The method according to any one of Embodiments 158 to 200, wherein the reference level for serum tryptase is above 1 micrograms/L, such as 1 micrograms/L to 4 micrograms/L, such as 1 micrograms/L to 2 micrograms/L, such as 1 micrograms/L to 1.5 micrograms/L, such as 1.1 micrograms/L, 1.2 micrograms/L, 1.3 micrograms/L, or 1.4 micrograms/L, or such as 1.5 micrograms/L to 2 micrograms/L, such as 1.5 micrograms/L, 1.6 micrograms/L, 1.7 micrograms/L, 1.8 micrograms/L, or 1.9 micrograms/L, or such as 2 micrograms/L to 4 micrograms/L, such as 2 micrograms/L to 3.5 micrograms/L, such as 2.1 micrograms/L to 2.5 micrograms/L, such as 2.2 micrograms/L, such as 2.3 micrograms/L, or such as 2.4 micrograms/L, or such as 2.5 micrograms/L to 3.5 micrograms/L, such as 2.6 micrograms/L, 2.47 micrograms/L, 2.48 micrograms/L, 2.49 micrograms/L, or such as 3 micrograms/L to 3.5 micrograms/L, such as 3.1 micrograms/L, 3.2 micrograms/L, 3.3 micrograms/L, 3.4 micrograms/L, or such as from 3.5 micrograms/L to 4 micrograms/L, such as such as 3.6 micrograms/L, 3.7 micrograms/L, 3.8 micrograms/L, 3.9 micrograms/L, or such as above 4 micrograms/L, such as from 4 micrograms/L to 8 micrograms/L, such as 5 micrograms/L, 6 micrograms/L, 7 micrograms/L, or above 8 micrograms/L such as 9 micrograms/L, or above.
Embodiment 202. The method according to any one of Embodiments 158 to 201, wherein the reference level for serum tryptase is about 2 micrograms/L.
Embodiment 203. The method according to any one of Embodiments 158 to 202, wherein the reference level for serum tryptase is about 3 micrograms/L.
Embodiment 204. The method according to any one of Embodiments 158 to 203, wherein the reference level for serum tryptase is about 3.5 micrograms/L.
Embodiment 205. The method according to any one of Embodiments 158 to 204, wherein the reference level for serum total IgE is 0.35 kU/L or higher, such as from 0.35 kU/L to 100 kU/L, such as 25 kU/L, such as 50 kU/L, or such as 75 kU/L, or such as from 100 kU/L to 150 kU/L, such as 125 kU/L, or such as from 150 kU/L to 600 kU/L, such as from 150 kU/L to 250 kU/L, such as 175 kU/L, or such as 200 kU/L, or such as 225 kU/L, or such as from 250 kU/L to 600 kU/L, such as 275 kU/L, or such as 300 kU/L, or such as 325 kU/L, or such as from 350 kU/L to 600 kU/L, such as 375 kU/L, or such as 400 kU/L, or such as from 400 kU/L to 600 kU/L, such as 425 kU/L, such as 450 kU/L, such as 450 kU/L, such as 460 kU/L, such as 461 kU/L, such as 462 kU/L, such as 463 kU/L, such as 464 kU/L, such as 465 kU/L, such as 466 kU/L, such as 467 kU/L, such as 468 kU/L, such as 469 kU/L, such as 470 kU/L, such as 475 kU/L, such as 480 kU/L, such as 490 kU/L, or such as from 500 kU/L to 600 kU/L, such as 525 kU/L, such as 550 kU/L, or such as 575 kU/L, or above 600 kU/L.
Embodiment 206. The method according to any one of Embodiments 158 to 205, wherein the reference level for serum total IgE is about 0.35 kU/L.
Embodiment 207. The method according to any one of Embodiments 158 to 206, wherein the reference level for serum total IgE is about 186 kU/L.
Embodiment 208. The method according to any one of Embodiments 158 to 207, wherein the reference level for serum total IgE is about 464 kU/L.
Embodiment 209. The method according to any one of Embodiments 158 to 208, wherein step a) further includes a step detecting whether a biological sample obtained from said subject has one or more of:
   i. A single nucleotide polymorphism (SNP) selected from the group consisting of the single nucleic acid polymorphisms located in the chromosome 17q12-21 region and/or;
   ii. A SNP in linkage disequilibrium with one or more SNPs as defined in i. above;
And further, wherein the step b) is selecting said subject for treatment with an immunotherapy product and/or predicting a beneficial response to treatment with an immunotherapy product in said subject, if either the level of one or more markers of step a) is increased or above a reference level, or one or more SNPs of step d) are detected, or both.
Embodiment 210. The method according to any one of Embodiments 158 to 209, wherein step a) further includes a step detecting whether a biological sample obtained from said subject has
   i. one or more of a SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10), or a complementary SNP thereof, or
   ii. A SNP in linkage disequilibrium with one or more SNPs as defined in i. above.
Embodiment 211. The method according to any one of Embodiments 158 to 210, wherein step a) further includes a step detecting whether a biological sample obtained from said subject has one or more of a SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10), or a complementary SNP thereof.
Embodiment 212. The method according to any one of Embodiments 158 to 211, wherein the immunotherapy product is defined as in any one of the previous Embodiments 22 to 49.
Embodiment 213. The method according to any one of the preceding Embodiments 158 to 212, wherein the immunotherapy product is an allergen-specific immunotherapy product.
Embodiment 214. The method according to any one of the preceding Embodiments 158 to 213, wherein said subject is defined by any one of Embodiments 57 to 89.
Embodiment 215. The method according to any one of the preceding Embodiments 158 to 214, wherein said atopic disease is defined by any one of the preceding Embodiments 67 to 89.
Embodiment 216. The method according to any one of the preceding Embodiments 158 to 215, wherein the method is performed to obtain a response to treatment as defined by any one of Embodiments 90 to 101.
Embodiment 217. A method of treating, preventing, delaying onset, or reducing risk of an atopic disease, such as asthma, in a subject in need thereof, said method comprising:
   a) Obtaining and/or providing a biological sample from said subject; and
   b) Quantifying the level of one or more Th2 markers selected from the group consisting of eosinophils, eosinophil cationic protein (ECP), tryptase, periostin, and total IgE;
   c) Identifying or selecting said subject for treatment with an immunotherapy product and/or predicting a beneficial response to treatment with an immunotherapy product in said subject, if the level of one or more markers of step a) is increased or above a reference level; and
   d) Administering an immunotherapy product to said subject identified or selected according to step c).
Embodiment 218. The method according to Embodiment 217, wherein the immunotherapy product is defined as in any one of Embodiments 22 to 56.
Embodiment 219. The method according to any one of the Embodiments 217 to 218, wherein the immunotherapy product is an allergen-specific immunotherapy product.
Embodiment 220. The method according to any one of the Embodiments 217 to 219, wherein the Th2 markers are defined by any one of Embodiments 161 to 190.
Embodiment 221. The method according to any one of Embodiments 217 to 220, wherein the reference level is defined by any one of Embodiments 160 to 167 and 192 to 208.
Embodiment 222. The method according to any one of Embodiments 217 to 221, wherein step a) further includes a step detecting whether a biological sample obtained from said subject has one or more of:
   i. A single nucleotide polymorphism (SNP) selected from the group consisting of the single nucleic acid polymorphisms located in the chromosome 17q12-21 region and/or;
   ii. A SNP in linkage disequilibrium with one or more SNPs as defined in i. above;
And further, wherein the step b) is selecting said subject for treatment with an immunotherapy product and/or predicting a beneficial response to treatment with an immunotherapy product in said subject, if either the level of one or more markers of step a) is increased or above a reference level, or one or more SNPs of step d) are detected, or both.
Embodiment 223. The method according to any one of Embodiments 217 to 222, wherein step a) further includes a step detecting whether a biological sample obtained from said subject has
   i. one or more of a SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10), or a complementary SNP thereof, or
   ii. A SNP in linkage disequilibrium with one or more SNPs as defined in i. above.
Embodiment 224. The method according to any one of Embodiments 217 to 223, wherein step a) further includes a step detecting whether a biological sample obtained from said subject has one or more of a SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10), or a complementary SNP thereof.
Embodiment 225. The method according to any one of Embodiments 217 to 224, wherein said subject is defined by any one of Embodiments 57 to 89.
Embodiment 226. The method according to any one of Embodiments 217 to 225, wherein said atopic disease is defined by any one of Embodiments 67 to 89.
Embodiment 227. The method according to any one of Embodiments 217 to 226 wherein the method is performed to obtain a response to treatment as defined by any one of Embodiments 90 to 101.
Embodiment 228. The method according to any one of Embodiments 217 to 227, wherein said method reduces the risk of asthma exacerbations and/or deterioration in symptoms and signs of asthma.
Embodiment 229. The method according to any one of Embodiments 217 to 228, wherein said method reduces the risk of moderate to severe asthma exacerbations.
Embodiment 230. The method according to any one of Embodiments 217 to 229, wherein said method reduces risk of experiencing one or more signs and symptoms of asthma and/or use of asthma medication.
Embodiment 231. The method according to any one of Embodiments 217 to 230, wherein said method reduces risk of experiencing and/or ameliorates one or more signs and symptoms of asthma.
Embodiment 232. The method according to any one of Embodiments 217 to 231, wherein said method reduces use of asthma medication (e.g. inhaled, systemic or oral corticosteroid) and/or β₂-adrenergic agonists.
Embodiment 233. The method according to Embodiments 217 to 232, wherein said method further comprises administration of at least one second active ingredient useful for treatment of asthma.
Embodiment 234. The method according to Embodiments 217 to 233, wherein said method further comprises administration of an antibody useful for treatment of asthma.
Embodiment 235. The method according to any one of Embodiments 217 to 234, wherein said method further comprises administration of one or more second agents for asthma treatment selected from the group consisting of corticosteroids (inhaled, systemic and/or oral), β₂-adrenergic agonists (short acting or long acting), muscarinic receptor antagonists (e.g. long acting muscarinic receptor antagonists, LAMA), anticholinergics, mast cell stabilizers, leukotriene antagonists or leukotriene modifiers, cromolyn sodium and methylxanthines.
Embodiment 236. The method according to any one of Embodiments 217 to 235, wherein said method further comprises administration of inhaled corticosteroid (e.g. budesonide, fluticasone propionate, fluticasone furoate or beclomethasone) and optionally a short acting β₂-adrenergic agonist (e.g. salbutamol or terbutaline).
Embodiment 237. The method according to any one of Embodiments 217 to 236, wherein said method further comprises administration of inhaled corticosteroid (e.g. budesonide) and optionally a short acting β₂-adrenergic agonist (e.g. salbutamol).
Embodiment 238. The method according to any one of Embodiments 217 to 237, wherein said method further comprises administration of inhaled corticosteroid (e.g. budesonide, fluticasone propionate, fluticasone furoate or beclomethasone) and a long acting β₂-adrenergic agonist (e.g. salmeterol, formeterol, or vilanterol) and optionally a short acting β₂-adrenergic agonist (e.g. salbutamol or terbutaline).
Embodiment 239. The method according to any one of Embodiments 217 to 238, wherein said method further comprises administration of inhaled corticosteroid (e.g. budesonide) and a long acting β₂-adrenergic agonist (e.g. salmeterol, formeterol, or vilanterol) and optionally a short acting β₂-adrenergic agonist (e.g. salbutamol).
Embodiment 240. An immunotherapy product for use in a method of treating, preventing or reducing risk of one or more atopic diseases, such as asthma, in a subject, said method comprising:
   a) Obtaining or providing a biological sample from a subject;
   b) Quantifying the level of one or more Th2 markers selected from the group consisting of eosinophils, eosinophil cationic protein (ECP), tryptase, periostin, and total IgE;
   c) Selecting and treating said subject with an immunotherapy product and/or predicting a beneficial response to treatment with an immunotherapy product in said subject, if the level of one or more markers of step b) is increased or above a reference level.
Embodiment 241. The immunotherapy product according to Embodiment 240, wherein the Th2 markers are defined by any one of Embodiments 168 to 191.
Embodiment 242. The immunotherapy product according to any one of Embodiments 240 to 241, wherein the reference level is defined by any one of Embodiments 160 to 167 and 192 to 208.
Embodiment 243. The immunotherapy product according to any one of Embodiments 240 to 242, wherein step b) further includes a step detecting whether a biological sample obtained from said subject has one or more of:
   i. A single nucleotide polymorphism (SNP) selected from the group consisting of the single nucleic acid polymorphisms located in the chromosome 17q12-21 region and/or;
   ii. A SNP in linkage disequilibrium with one or more SNPs as defined in i. above;
And further, wherein the step c) is selecting said subject for treatment with an immunotherapy product and/or predicting a beneficial response to treatment with an immunotherapy product in said subject, if either the level of one or more markers of step b) is increased or above a reference level, or one or more SNPs of step d) are detected, or both.
Embodiment 244. The immunotherapy product to any one of Embodiments 240 to 243, wherein step b) further includes a step detecting whether a biological sample obtained from said subject has
   i. one or more of a SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10), or a complementary SNP thereof, or a complementary SNP thereof, or
   ii. A SNP in linkage disequilibrium with one or more SNPs as defined in i. above.
Embodiment 245. The immunotherapy product according to any one of Embodiments 240 to 244, wherein step b) further includes a step detecting whether a biological sample obtained from said subject has one or more of a SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10) or a complementary SNP thereof.
Embodiment 246. The immunotherapy product according to any one of Embodiments 240 to 245, wherein said immunotherapy product is further defined by any one of the preceding Embodiments 22 to 49.
Embodiment 247. The immunotherapy product according to any one of the Embodiments 240 to 246, wherein said immunotherapy product is for allergen-specific immunotherapy.
Embodiment 248. The immunotherapy product according to any one of Embodiments 240 to 247, wherein said subject is further defined by any one of Embodiments 57 to 89.
Embodiment 249. The immunotherapy product according to any one of Embodiments 240 to 248, wherein said immunotherapy product is for allergen-specific immunotherapy, and comprises a house dust mite allergen extract for sublingual administration.
Embodiment 250. The immunotherapy product according to any one of Embodiments 240 to 249, wherein said immunotherapy product is for allergen-specific immunotherapy, and comprises a house dust mite allergen extract formulated as a fast dispersing tablet for sublingual administration.
Embodiment 251. The immunotherapy product according to any one of the Embodiments 240 to 250, wherein said immunotherapy product is for allergen-specific immunotherapy, and comprises a grass pollen allergen extract for sublingual administration.
Embodiment 252. The immunotherapy product according to any one of Embodiments 240 to 251, wherein said immunotherapy product is for allergen-specific immunotherapy, and comprises a grass pollen allergen extract formulated as a fast dispersing tablet for sublingual administration.
Embodiment 253. The immunotherapy product according to any one of the Embodiments 240 to 252, wherein said method is to obtain a response as defined according to any one of Embodiments 90 to 101.
Embodiment 254. The immunotherapy product according to any one of Embodiments 240 to 253, wherein said method further comprises simultaneous, subsequent or singular administration of at least one second active ingredient useful for treatment of asthma.
Embodiment 255. The immunotherapy product according to any one of Embodiments 240 to 253, wherein said method further comprises administration of one or more secondary agents for asthma treatment selected from the group consisting of corticosteroids (inhaled, systemic and/or oral) e.g. fluticasone propionate, fluticasone furoate, budesonide, beclomethasone, β₂-adrenergic agonists (short acting or long acting), muscarinic receptor antagonists (e.g. long acting muscarinic receptor antagonists), anticholinergics, mast cell stabilizers, leukotriene antagonists or leukotriene modifiers, cromolyn sodium and methylxanthines.
Embodiment 256. The immunotherapy product according to any one of Embodiments 240 to 255, wherein said method further comprises administration of inhaled corticosteroid (e.g. budesonide, fluticasone propionate, fluticasone furoate or beclomethasone) and optionally a short acting β₂-adrenergic agonist (e.g. salbutamol or terbutaline).
Embodiment 257. The immunotherapy product according to any one of Embodiments 240 to 256, wherein said method further comprises administration of inhaled corticosteroid (e.g. budesonide) and optionally a short acting β₂-adrenergic agonist (e.g. salbutamol).
Embodiment 258. The immunotherapy product according to any one of Embodiments 240 to 257, wherein said method further comprises administration of inhaled corticosteroid (e.g. budesonide, fluticasone propionate, fluticasone furoate or beclomethasone) and a long acting β₂-adrenergic agonist (e.g. salmeterol, formeterol, or vilanterol) and optionally a short acting β₂-adrenergic agonist (e.g. salbutamol or terbutaline).
Embodiment 259. The immunotherapy product according to any one of Embodiments 240 to 258, wherein said method further comprises administration of inhaled corticosteroid (e.g. budesonide) and a long acting β₂-adrenergic agonist (e.g. salmeterol, formeterol, or vilanterol) and optionally a short acting β₂-adrenergic agonist (e.g. salbutamol).
Embodiment 260. The immunotherapy product according to any one of Embodiments 240 to 259, for reducing risk of asthma exacerbations and/or reduce risk of deterioration of one or more symptoms and signs of asthma.
Embodiment 261. The immunotherapy product according to any one of Embodiments 240 to 260, for reducing risk of moderate to severe asthma exacerbations.
Embodiment 262. Use of an allergen and/or antibody in the manufacture of a medicament for the treatment of atopic disease such as asthma, wherein said treatment comprises:
   a) Quantifying the level of one or more Th2 markers selected from the group consisting of eosinophils, eosinophil cationic protein (ECP), tryptase, periostin, and total IgE;
   b) Selecting said subject for treatment with an immunotherapy product and/or predicting a beneficial response to treatment with an immunotherapy product in said subject, if the level of one or more markers of step a) is increased or above a reference level.
Embodiment 263. The use according to Embodiment 262, wherein said medicament is a immunotherapy product.
Embodiment 264. The use according to any one of Embodiments 262 and 263, wherein said medicament is an immunotherapy product defined according to any one of the Embodiments 22 to 49, 122 to 139 and 240 to 261.
Embodiment 265. The use according to any one of Embodiments 262 and 264, wherein the Th2 markers are defined by any one of Embodiments 168 to 191.
Embodiment 266. The use according to any one of Embodiments 262 and 265, wherein the reference level is defined by any one of Embodiments 160 to 167 and 192 to 208.
Embodiment 267. The use according to any one of Embodiments 262 and 266, wherein step a) further includes a step detecting whether a biological sample obtained from said subject has one or more of:
   i. A single nucleotide polymorphism (SNP) selected from the group consisting of the single nucleic acid polymorphisms located in the chromosome 17q12-21 region and/or;
   ii. A SNP in linkage disequilibrium with one or more SNPs as defined in i. above;
And further, wherein the step b) is selecting said subject for treatment with an immunotherapy product and/or predicting a beneficial response to treatment with an immunotherapy product in said subject, if either the level of one or more markers of step a) is increased or above a reference level, or one or more SNPs of step d) are detected, or both.
Embodiment 268. The method according to any one of Embodiments 262 and 267, wherein step a) further includes a step detecting whether a biological sample obtained from said subject has
   i. one or more of a SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10) or a complementary SNP thereof, or
   ii. A SNP in linkage disequilibrium with one or more SNPs as defined in i. above.
Embodiment 269. The method according to any one of Embodiments 262 and 268, wherein step a) further includes a step detecting whether a biological sample obtained from said subject has one or more of a SNPs selected from the group consisting of rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2) and rs7216389 (SEQ ID NO: 10) or a complementary SNP thereof.
Embodiment 270. A kit of parts comprising:
   a) Means for quantifying the level of one or more Th2 markers selected from the group consisting of eosinophils, eosinophil cationic protein (ECP), tryptase, periostin, and total IgE; and
   b) Instructions for use of said kit in combination with an immunotherapy product.
Embodiment 271. The method according to Embodiment 270, wherein the Th2 markers are defined by any one of Embodiments 168 to 191.
Embodiment 272. The method according to any one of Embodiments 270 to 271, wherein the reference level is defined by any one of Embodiments 160 to 167 and 192 to 208.
Embodiment 273. The kit of parts according to any one of Embodiments 270 to 272, further comprising means for detection of one or more SNPs as defined in Embodiments 3 to 21.
Embodiment 274. The kit of parts according to any one of Embodiments 270 to 273, wherein the immunotherapy product is defined according to any one of the Embodiments 22 to 49, 122 to 139 and 240 to 261.
Embodiment 275. The kit of parts according to any one of Embodiments 270 to 274, further comprising means for obtaining one or more biological samples.
Embodiment 276. The kit of parts according to any one of Embodiments 270 to 275, further comprising means for diagnosis of allergy or sensitization to an allergen.
Embodiment 277. The kit of parts according to any one of Embodiments 270 to 276, further comprising means for a skin prick test or for measurement of IgE in a biological sample.
Embodiment 278. A kit of parts comprising:
   a) An immunotherapy product, and
   b) Instructions for use of said immunotherapy product in combination with a method as defined any of Embodiments 1 to 121, and 158 to 239;
Embodiment 279. The kit of parts according to Embodiment 278, wherein the Th2 markers are defined by any one of Embodiments 168 to 191.
Embodiment 280. The method according to any one of the Embodiments 278 to 279, wherein the reference level is defined by any one of Embodiments 160 to 167 and 192 to 208.
Embodiment 281. The kit of parts according to any one of Embodiments 278 to 280, wherein the immunotherapy product is defined according to any one of Embodiments 22 to 49, 122 to 139 and 240 to 261.
Embodiment 282. The kit of parts according to any one of Embodiments 278 to 281, further comprising means for quantifying the level of one or more Th2 markers selected from the group consisting of eosinophils, eosinophil cationic protein (ECP), tryptase, periostin and total IgE.
Embodiment 283. The kit of parts according to any one of Embodiments 278 to 282, further comprising means for quantifying the level of one or more Th2 markers selected from the group consisting of eosinophils, eosinophil cationic protein (ECP), tryptase, and periostin.
Embodiment 284. The kit of parts according to any one of Embodiments 278 to 283, further comprising means or instructions for use of said immunotherapy product in combination with a method comprising a step of detection of one or more SNPs as defined in Embodiments 3 to 21.
Embodiment 285. The kit of parts according to any one of Embodiments 278 to 284, further comprising means for detection of one or more SNPs as defined in Embodiments 3 to 21.
Embodiment 286. The kit of parts according to any one of Embodiments 278 to 285, further comprising means for obtaining one or more biological samples.
Embodiment 287. Use of one or more Th2 markers selected from the group consisting of eosinophils, eosinophil cationic protein (ECP), tryptase, periostin, and total IgE for predicting responsiveness of a subject to treatment with an immunotherapy product.
Embodiment 288. The use according to Embodiment 287, wherein the Th2 markers are defined by any one of Embodiments 168 to 191.
Embodiment 289. The use according to any one of Embodiments 287 to 288, further comprising the use of a reference level as defined by any one of Embodiments 160 to 167 and 192 to 208.
Embodiment 290. The use according to any one of Embodiments 287 to 289, wherein the immunotherapy product is defined by any one of Embodiments 22 to 49, 122 to 139 and 240 to 261.
Embodiment 291. The use according to any one of Embodiments 287 to 290, wherein the immunotherapy product is an allergen-specific immunotherapy product.
Embodiment 292. Use of one or more Th2 markers selected from the group consisting of eosinophils, eosinophil cationic protein (ECP), tryptase, periostin, and total IgE;
   3. for selecting a subject for treatment with an immunotherapy product.
Embodiment 293. The use according to Embodiment 292, wherein the Th2 markers are defined by any one of Embodiments 168 to 191.
Embodiment 294. The use according to any one of the Embodiments 292 to 293, further comprising the use of a reference level as defined by any one of Embodiments 160 to 167 and 192 to 208.
Embodiment 295. The use according to any one of the Embodiments 292 to 294, wherein the immunotherapy product is defined by any one of Embodiments 22 to 49, 122 to 139 and 240 to 261.
Embodiment 296. The use according to any one of Embodiments 292 to 295, wherein the immunotherapy product is an allergen-specific immunotherapy product.

### EXAMPLES

### Example 1

The present example discloses a clinical trial wherein house dust mite allergen extract was tested for treatment of asthmatic subjects.

The study was a double-blind, randomized, placebo-controlled trial that included 834 adults (mean age, 33 years [range, 17-83]; women, 48%, Caucasian descent, 99%) with house dust mite (HDM) allergy related asthma and rhinitis (more details described in Virchow et al. 2016). Adults included in the study were tested positive for HDM-specific serum IgE and HDM sensitivity in a skin prick test. They had a clinical history of allergic asthma and allergic rhinitis of more than 1 year with HDM being considered the major trigger. The asthma symptoms of the trial participants were not well controlled by inhaled corticosteroids (ICS) at inclusion.

The trial participants were randomized to receive treatment with HDM allergen extract in a dose of 6 SQ-HDM (n=275), or 12 SQ-HDM (n=282), or no HDM allergen extract (i.e. placebo) (n=277) administered sublingually as a fast dispersing tablet. During the first treatment period with placebo or HDM allergen extract, patients were additionally treated with ICS provided as budesonide powder for inhalation in strengths of 100 to 200 microgram per dose for maintenance treatment of asthma, and short acting β₂-agonist (SABA) administered as salbutamol for inhalation in strength 200 microgram per dose as needed for the control of asthma symptoms throughout the trial. After 7 to 12 months of treatment, a reduction and withdrawal period started, including 3 months of daily ICS use reduced to 50% followed by 3 months of complete ICS withdrawal.

The primary endpoint of the clinical trial was the time to the first moderate or severe asthma exacerbation during the ICS reduction and withdrawal period. Baseline values of the symptom scores, medication use and lung function score were obtained by observing and recording these scores for each patient in the last 2 weeks prior to the ICS reduction and withdrawal period.

Criteria a) to d) defined a moderate exacerbation:
a) Nocturnal awakening(s) due to asthma requiring SABA use for at least 2 consecutive nights or an increase of minimum 0.75 in daily symptom score from baseline value on at least 2 consecutive days.
b) An increase from the baseline value in occasions of SABA use on at least 2 consecutive days (a minimum increase of 4 puffs per day).
c) ≥20% decrease in peak expiratory flow (PEF) from baseline value on at least 2 consecutive mornings or evenings or a 20% decrease in FEV₁ from baseline value.
d) Visit to the emergency room or unscheduled visit to the trial centre for asthma treatment not requiring systemic corticosteroids.

Criteria e) to f) defined a severe exacerbation:
e) Need of systemic corticosteroids for the treatment of asthma symptoms for at least 3 days
f) Emergency room visit because of asthma, requiring systemic corticosteroids or hospitalization for more than 12 hours because of asthma.

The treatment with house dust mite allergen extract (6 SQ-HDM and 12 SQ-HDM tablets) significantly reduced the risk of experiencing a moderate or severe asthma exacerbation in the 6-month efficacy assessment period compared to placebo (hazard ratio (HR)=0.72 [0.52-0.99], p=0.045, for 6 SQ-HDM; HR=0.69 [0.5-0.96], p=0.03, for 12 SQ-HDM).

The secondary endpoint was the time to first asthma exacerbation with deterioration in asthma symptoms (daytime symptoms or nocturnal awakenings requiring SABA administered as salbutamol for inhalation in strength 200 microgram per dose) to control asthma symptoms. Compared with placebo, there was a reduced risk of an exacerbation with deterioration in asthma symptoms, HR=0.72 [0.49-1.02] for the 6SQ-HDM group, P=0.11, and HR=0.64 [0.42-0.96] for the 12SQ-HDMgroup, P=0.03.

The 6 SQ-HDM and 12 SQ-HDM tablets significantly reduced the risk of experiencing a moderate or severe asthma exacerbation in the 6-month efficacy assessment period compared to placebo. Further, the active treatment with 6 SQ-HDM and 12SQ-HDM tablets significantly decreased the risk of having exacerbation with deterioration in asthma symptoms compared to placebo.

For further analysis, a subgroup of 664 trial participants was defined which had completed the full study without any major protocol deviations that might influence the primary endpoint of the trial (a so-called “per protocol” group of participants).

### Genotyping Study of Participants

A genotyping study was performed on a subset of the per protocol participants, for which a) DNA samples were available, and b) informed consent for genetic studies had been given (n=526). SNP typing was done with the Infinium Global Screening Assay (GSA) (Illumina, Inc.). A list of 21 SNPs previously associated with asthma and/or allergy and that were present on the GSA chip was compiled, see Table 2 below (Moffatt et al. 2007, Moffatt et al. 2010, Bønnelykke et al. 2014, Ferreira et al. 2014).

TABLE 2
A compiled list of 21 SNP alleles associated with risk of asthma

| SEQ | | | | | Nearest | | |
|---|---|---|---|---|---|---|---|
| ID | | | Position | Associated | ENCODE | Risk | |
| NO | SNP ID | Chromosome | (GRCh37) | trait | gene | Allele | Literature |
| 18 | rs10197862 | 2 | 102966549 | Asthma and | IL1RL1 | A | Ferreira |
| | | | | rhinitis | | | 2014 |
| 19 | rs3771166 | 2 | 102986222 | Asthma | IL18R1 | G | Moffatt |
| | | | | | | | 2010 |
| 20 | rs4833095 | 4 | 38799710 | Asthma and | TLR1 | T | Ferreira |
| | | | | rhinitis | | | 2014 |
| 21 | rs1837253 | 5 | 110401872 | Asthma and | TSLP | C | Ferreira |
| | | | | rhinitis | | | 2014 |
| 22 | rs1438673 | 5 | 110467499 | Asthma and | WDR36 | C | Ferreira |
| | | | | rhinitis | | | 2014 |
| 23 | rs2073643 | 5 | 131723288 | Asthma | SLC22A5 | T | Moffatt |
| | | | | | | | 2010 |
| 24 | rs1295686 | 5 | 131995843 | Asthma | IL13 | T | Moffatt |
| | | | | | | | 2010 |
| 25 | rs9273349 | 6 | 32625869 | Asthma | HLA-DQ | C | Moffatt |
| | | | | | | | 2010 |
| 26 | rs6967330 | 7 | 105658451 | Childhood | CDHR3 | A | Bønnelykke |
| | | | | asthma | | | 2014 |
| 27 | rs7009110 | 8 | 81291879 | Asthma and | ZBTB10 | T | Ferreira |
| | | | | rhinitis | | | 2014 |
| 28 | rs72699186 | 9 | 6175855 | Asthma and | IL33 | T | Ferreira |
| | | | | rhinitis | | | 2014 |
| 29 | rs1342326 | 9 | 6190076 | Asthma | IL33 | C | Moffatt |
| | | | | | | | 2010 |
| 30 | rs928413 | 9 | 6213387 | Childhood | IL33 | G | Bønnelykke |
| | | | | asthma | | | 2014 |
| 31 | rs11071559 | 15 | 61069988 | Asthma | RORA | C | Moffatt |
| | | | | | | | 2010 |
| 32 | rs17294280 | 15 | 67468285 | Asthma and | SMAD3 | G | Ferreira |
| | | | | rhinitis | | | 2014 |
| 33 | rs62026376 | 16 | 11228712 | Asthma and | CLEC16A | C | Ferreira |
| | | | | rhinitis | | | 2014 |
| 3 | rs2305480 | 17 | 38062196 | Asthma | GSDMB | G | Moffatt |
| | | | | | | | 2007; |
| | | | | | | | Moffatt |
| | | | | | | | 2010; |
| | | | | | | | Bønnelykke |
| | | | | | | | 2014 |
| 10 | rs7216389 | 17 | 38069949 | Asthma | ORMDL3 | T | Moffatt |
| | | | | | | | 2007 |
| 8 | rs3894194 | 17 | 38121993 | Asthma | GSDMA | A | Moffatt |
| | | | | | | | 2010 |
| 34 | rs7212938 | 17 | 38122680 | Asthma and | GSDMA | G | Ferreira |
| | | | | rhinitis | | | 2014 |
| 35 | rs2284033 | 22 | 37534034 | Asthma | IL2RB | G | Moffatt |
| | | | | | | | 2010 |

The genotypes of these SNPs were extracted from the GSA data using the software Plink v1.9 (Purcell et al. 2007). Each SNP was then assessed for its effect on the risk of experiencing a moderate or severe asthma exacerbation in either of the three treatment groups (Placebo, 6 SQ-HDM and 12 SQ-HDM).

The effect of each SNP on the risk to experience an asthma exacerbation was assessed with a Cox proportional hazard (CoxPH) regression analysis, taking into account the days to first asthma exacerbation and including an interaction between treatment group and SNP genotype. From this model, we then derived the hazard ratio of experiencing an asthma exacerbation when having a certain genotype of a SNP compared to another genotype within each treatment group. In addition, we derived the hazard ratio between the treatment groups within each SNP genotype. The hazard ratio between two subpopulations indicates the risk of experiencing an exacerbation at any given day during the ICS withdrawal/reduction phase of one subpopulation relative to the other subpopulation. If the hazard ratio is larger than 1, the risk is increased, whereas if the hazard ratio is smaller than 1, the risk is decreased relative to the other subpopulation. The 95% confidence interval indicates the statistical uncertainty of the model. If the confidence interval does not overlap with 1 (the line representing equal risk between the groups), the difference in risk between the two compared subpopulations is statistically significant (P<0.05).

In addition, logistic regression was employed to test the effect of a SNP on the odds to experience at least one moderate to severe asthma exacerbation within the 6 months of ICS reduction/withdrawal. In the logistic regression model, the response variable was the binary response for each subject of whether asthma exacerbation was experienced in the whole 6 month period or not. The SNP genotype was included in an interaction term with the treatment group as a fixed effect.

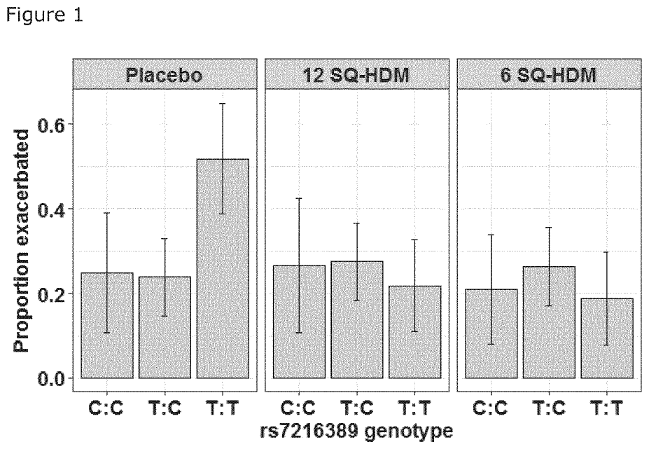

Standing out as one SNP with markedly increased risk of experiencing an asthma exacerbation in the Placebo group was the SNP rs7216389 of the 17q12-21 region (see FIG. 1 and Table 3). The homozygote T:T genotype of rs7216389 is known to be associated with the risk to develop asthma, especially in childhood. In our clinical trial population, the subpopulation carrying the T:T genotype showed a large treatment effect at both the 12 SQ-HDM and the 6 SQ-HDM dose (comparisons T:T.12SQ-T:T.Placebo and T:T.6SQ-T:T.Placebo in Table 3). The asthma exacerbation risk and the treatment effect associated with the T:T genotype are also apparent from the barplot shown in FIG. 1. The bars show the proportion of each subpopulation (defined by SNP genotype and treatment group) who experienced an asthma exacerbation. Error bars indicate the 95% confidence interval calculated as prop±1.96*\sqrt{prop*(1−prop)/N}, where prop=N_{exacerbated}/N. Among the 21 SNPs tested and listed in Table 2, only rs7216389 and rs2305480 (both located on chromosome 17) showed a consistent significant association with treatment effect in the trial cohort.

Based on the data of the present study, the homozygote T:T genotype of rs7216389 (SEQ ID NO: 10) was associated with a more than 2-fold higher risk of experiencing a moderate or severe asthma exacerbation in the Placebo group. See Table 3: The hazard ratio of experiencing an asthma exacerbation in the Placebo group was 2.29 (p=0.03) for carriers of the T:T genotype compared to carriers of the C:C genotype. Similarly, the hazard ratio was 2.5 (p=0.002) for carriers of the T:T genotype compared to carriers of the T:C genotype. This means that at any given day during the 6 month ICS reduction/withdrawal period, Placebo subjects carrying the T:T genotype had a more than 2-fold risk of experiencing a moderate to severe asthma exacerbation compared to Placebo subjects carrying other rs7216389 (SEQ ID NO: 10) genotypes.

Most notably, within the T:T genotype, a large treatment effect was observed with a 66% reduction in the risk of experiencing a moderate to severe exacerbation for 12 SQ-HDM treatment (hazard ratio: 0.34, p=0.002) and a risk reduction of 71% for 6 SQ-HDM treatment (hazard ratio: 0.29, p=0.001). The results of the logistic regression confirmed the findings, see Table 4.

These results show that treatment with allergen specific immunotherapy can reduce the effects of a genetic predisposition to an increased asthma exacerbation risk.

The subgroup of subjects used for the genotyping above, were further genotyped with respect to 8 other alleles of SNPs, which were all located in the chromosomal region 17q12-21 and were associated with risk of developing asthma (see Stein et al. 2018). These were further tested for their association with treatment effect and risk of experiencing a moderate or severe asthma exacerbation in the same trial cohort as described above. The majority of the tested SNPs showed a significant association with effect of treatment and a trend towards increased risk to exacerbate, see the tables below demonstrating results of CoxPH regression analysis and logistic regression analysis for SNPs rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2).

TABLE 5
Hazard ratios and odds ratios for rs907092

| rs907092 | Hazard Ratio | lwr | upr | p-value |
|---|---|---|---|---|
| G:G.Placebo - A:G.Placebo | 2.99 | 1.65 | 5.42 | 0.0003 |
| G:G.Placebo - A:A.Placebo | 2.07 | 0.96 | 4.48 | 0.06 |
| G:G.12SQ - G:G.Placebo | 0.38 | 0.21 | 0.70 | 0.002 |
| G:G.6SQ - G:G.Placebo | 0.26 | 0.12 | 0.53 | 0.0003 |
| rs907092 | Odds ratio | lwr | upr | p-value |
| G:G.Placebo - A:G.Placebo | 4.32 | 2.08 | 8.95 | 0.0001 |
| G:G.Placebo - A:A.Placebo | 2.92 | 1.14 | 7.50 | 0.03 |
| G:G.12SQ - G:G.Placebo | 0.28 | 0.13 | 0.60 | 0.001 |
| G:G.6SQ - G:G.Placebo | 0.18 | 0.08 | 0.43 | 0.0001 |

TABLE 6
Hazard ratios and odds ratios for rs9303277

| rs9303277 | Hazard Ratio | lwr | upr | p-value |
|---|---|---|---|---|
| C:C.Placebo - C:T.Placebo | 2.51 | 1.42 | 4.42 | 0.001 |
| C:C.Placebo - T:T.Placebo | 2.22 | 1.05 | 4.70 | 0.04 |
| C:C.12SQ - C:C.Placebo | 0.38 | 0.19 | 0.73 | 0.004 |
| C:C.6SQ - C:C.Placebo | 0.26 | 0.21 | 0.57 | 0.001 |
| rs9303277 | Odds ratio | lwr | upr | p-value |
| C:C.Placebo - C:T.Placebo | 3.44 | 1.66 | 7.09 | 0.001 |
| C:C.Placebo - T:T.Placebo | 3.11 | 1.23 | 7.86 | 0.02 |
| C:C.12SQ - C:C.Placebo | 0.29 | 0.13 | 0.65 | 0.003 |
| C:C.6SQ - C:C.Placebo | 0.19 | 0.07 | 0.47 | 0.0004 |

TABLE 7
Hazard ratios and odds ratios for rs8069176

| rs8069176 | Hazard Ratio | lwr | upr | p-value |
|---|---|---|---|---|
| G:G.Placebo - G:A.Placebo | 1.93 | 1.09 | 3.40 | 0.02 |
| G:G.Placebo - A:A.Placebo | 2.07 | 0.92 | 4.70 | 0.08 |
| G:G.12SQ - G:G.Placebo | 0.47 | 0.26 | 0.86 | 0.01 |
| G:G.6SQ - G:G.Placebo | 0.39 | 0.20 | 0.76 | 0.006 |
| rs8069176 | Odds ratio | lwr | upr | p-value |
| G:G.Placebo - G:A.Placebo | 2.57 | 1.28 | 5.17 | 0.01 |
| G:G.Placebo - A:A.Placebo | 2.77 | 1.05 | 7.28 | 0.04 |
| G:G.12SQ - G:G.Placebo | 0.38 | 0.18 | 0.79 | 0.01 |
| G:G.6SQ - G:G.Placebo | 0.30 | 0.14 | 0.67 | 0.003 |

TABLE 8
Hazard ratios and odds ratios for rs2305480

| rs2305480 | Hazard Ratio | lwr | upr | p-value |
|---|---|---|---|---|
| G:G.Placebo - G:A.Placebo | 2.06 | 1.16 | 3.66 | 0.01 |
| G:G.Placebo - A:A.Placebo | 2.13 | 0.94 | 4.83 | 0.07 |
| G:G.12SQ - G:G.Placebo | 0.46 | 0.25 | 0.83 | 0.01 |
| G:G.6SQ - G:G.Placebo | 0.38 | 0.20 | 0.74 | 0.004 |
| rs2305480 | Odds ratio | lwr | upr | p-value |
| G:G.Placebo - G:A.Placebo | 2.75 | 1.36 | 5.57 | 0.005 |
| G:G.Placebo - A:A.Placebo | 2.85 | 1.09 | 7.50 | 0.03 |
| G:G.12SQ - G:G.Placebo | 0.37 | 0.18 | 0.77 | 0.01 |
| G:G.6SQ - G:G.Placebo | 0.29 | 0.13 | 0.65 | 0.002 |

TABLE 9
Hazard ratios and odds ratios for rs11078927

| rs11078927 | Hazard Ratio | lwr | upr | p-value |
|---|---|---|---|---|
| C:C.Placebo - C:T.Placebo | 2.09 | 1.18 | 3.72 | 0.01 |
| C:C.Placebo - T:T.Placebo | 2.14 | 0.94 | 4.83 | 0.07 |
| C:C.12SQ - C:C.Placebo | 0.46 | 0.25 | 0.83 | 0.01 |
| C:C.6SQ - C:C.Placebo | 0.38 | 0.20 | 0.74 | 0.004 |
| rs11078927 | Odds ratio | lwr | upr | p-value |
| C:C.Placebo - C:T.Placebo | 2.80 | 1.38 | 5.66 | 0.004 |
| C:C.Placebo - T:T.Placebo | 2.85 | 1.09 | 7.50 | 0.03 |
| C:C.12SQ - C:C.Placebo | 0.37 | 0.18 | 0.77 | 0.01 |
| C:C.6SQ - C:C.Placebo | 0.29 | 0.13 | 0.65 | 0.002 |

TABLE 10
Hazard ratios and odds ratios for rs2290400

| rs2290400 | Hazard Ratio | lwr | upr | p-value |
|---|---|---|---|---|
| T:T.Placebo - T:C.Placebo | 2.20 | 1.25 | 3.87 | 0.01 |
| T:T.Placebo - C:C.Placebo | 2.19 | 1.03 | 4.63 | 0.04 |
| T:T.12SQ - T:T.Placebo | 0.38 | 0.20 | 0.73 | 0.004 |
| T:T.6SQ - T:T.Placebo | 0.31 | 0.15 | 0.65 | 0.002 |
| rs2290400 | Odds ratio | lwr | upr | p-value |
| T:T.Placebo - T:C.Placebo | 2.90 | 1.41 | 5.98 | 0.004 |
| T:T.Placebo - C:C.Placebo | 3.00 | 1.20 | 7.52 | 0.02 |
| T:T.12SQ - T:T.Placebo | 0.30 | 0.13 | 0.66 | 0.003 |
| T:T.6SQ - T:T.Placebo | 0.23 | 0.09 | 0.56 | 0.001 |

In agreement with the results shown above for rs7216389 (SEQ ID NO: 10), a large treatment effect was observed in trial subpopulations carrying the risk genotype of any of these SNPs (rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), rs2290400 (SEQ ID NO: 2)), resulting in a reduced risk to exacerbate of at least 50% (indicated by hazard ratios <0.5 in tables above) when comparing treatment with 12 SQ-HDM or 6 SQ-HDM, respectively, with Placebo within the subpopulation carrying the risk genotype.

Table 11 below shows the proportion of trial participants having a genoptype associated with effect of treatment in the population of subjects included in the genotyping study.

TABLE 11
The proportion of subjects of effect genotype

| | | Proportion of trial participants |
|---|---|---|
| SNP ID | Effect genotype | used for genotyping study |
| rs907092 | G:G | 36% |
| rs9303277 | C:C | 30% |
| rs8069176 | G:G | 37% |
| rs2305480 | G:G | 38% |
| rs11078927 | C:C | 37% |
| rs2290400 | T:T | 31% |
| rs7216389 | T:T | 30% |

In the data obtained from the trial cohort, there was no significant association found between the genotypes and effect of treatment for the two SNPs rs3894194 (SEQ ID NO: 4) and rs3859192 (SEQ ID NO: 7), and also no increased risk of experiencing a moderate to severe asthma exacerbation.

Nucleotides that are in close proximity on a chromosome are genetically linked, because they are not inherited independently. Genetic linkage can be estimated by calculating linkage disequilibrium (LD) scores in terms of r² between pairs of SNPs. LD analysis shows that some SNPs in the 17q12-21 region are highly linked in certain populations (Stein et al. 2018). The analysis by Stein et al. also suggests a sudden break of linkage upstream of rs3894194 (SEQ ID NO: 8) in some populations. We performed an LD analysis on our trial population by calculating pairwise r² values between the 9 SNPs located in the 17q12-21 region. The r² between two SNPs A and B is defined as r²(pₐ,p_{b},p_{ab})=(p_{ab}−pₐp_{b})²/(pₐ(1−p_{b}(1−p_{b})), where pₐ is the frequency of allele a of SNP A, p_{b} is the frequency of allele b of SNP B, and p_{ab} is the frequency of haplotypes (i.e. co-occurrence on one chromosome) with allele a at SNP A and b at SNP B (Hill and Robertson 1968). r² can assume values between 0 and 1, where values close to 1 for two loci (or SNPs) mean that the two loci in question are in strong genetic linkage and that alleles are inherited together. The results of our LD analysis shown in Table 12 demonstrate that for all the SNPs upstream and including rs7216389 (SEQ ID NO: 10), all pairwise r² values are >0.75, indicating genetic linkage (or linkage disequilibrium), whereas the two SNPs located downstream of rs7216389 (SEQ ID NO: 10), which are rs3894194 (SEQ ID NO: 8) and rs3859192 (SEQ ID NO: 7), show r² values <0.5 to all other 17q12-21 SNPs included in the analysis, suggesting weaker genetic linkage (i.e. no linkage disequilibrium in the trial population).

### Th2 Marker Study of Participants

A number of candidate markers were selected for assessment of association with treatment effect in the “per protocol” group of participants of the trial population described above. Among the markers were the number (or counted number) of blood eosinophils, eosinophil cationic protein (ECP), tryptase, periostin, and total IgE. Eosinophil counts were measured in patient blood as part of a standard complete blood count in the context of trial safety assessments. ECP, tryptase and total IgE were measured in patient serum by ImmunoCAP (Thermo Scientific). The lower limit of detection was 0.5 μg/L for ECP, 1 μg/L for tryptase and 1 kU/L for total IgE. Serum periostin levels were measured by ELISA (Human Periostin/OSF-2 DuoSet ELISA, R&D Systems, DY3548B). All measurements were performed on samples taken in the screening phase of the trial, i.e. before randomization of the trial participants into treatment groups.

In order to define a Th2-high subpopulation among the trial participants, we divided Th2 marker values into “high” or “normal”, where “high” was defined as a value which fell among the top 20% of the measured values for this marker for all trial samples, and “normal” were the values which fell among the remaining 80% of the measured values for this marker for all trial samples. See Table 13 for values that defined the Th2 high threshold values for each marker.

TABLE 13
Th2 marker values in a trial population of allergic asthmatics

| | | |
|---|---|---|
| | Upper 20% | Geometric mean in |
| Th2 marker | threshold | trial population |

| | | | | |
|---|---|---|---|---|
| Serum ECP | 29.4 | μg/L | 14.4 | μg/L |
| Blood Eosinophil | 400 | cells/μL | 200 | cells/μL |
| count | | | | |
| Serum Periostin | 27.8 | ng/mL | 21.0 | ng/mL |
| Serum Tryptase | 3.5 | μg/L | 2.2 | μg/L |
| Total IgE | 464 | kU/L | 186 | kU/L |

We assessed the effect of each Th2 marker on the risk to experience an asthma exacerbation in each of the three treatment groups. The statistical analysis was performed on the per protocol analysis set (defined above). Similar to the analysis described above in the genotyping study, the effect of each Th2 marker on the risk to experience an asthma exacerbation was assessed with a CoxPH regression analysis, taking into account the days to first asthma exacerbation and including an interaction between treatment group and Th2 marker level (“high” or “normal”). In addition, we used logistic regression to test the effect of a Th2 marker on the odds to experience at least one moderate to severe asthma exacerbation within the 6 months of ICS reduction/withdrawal. In the logistic regression model, the response variable was the binary response for each subject of whether asthma exacerbation was experienced in the whole 6 month period or not. The Th2 marker level was included in an interaction term with the treatment group.

Of the five markers tested, only total IgE did not result in a significant interaction with the treatment group. For Placebo-treated trial participants, CoxPH regression analysis resulted in hazard ratios of 2.09 for ECP, 1.74 for eosinophils, 2.13 for periostin and 1.88 for tryptase when comparing the group of Th2 marker “high” subjects with “normal” subjects (see Table 14, Table 15, Table 16, Table 17 and Table 18). This means that the risk of experiencing an asthma exacerbation at any given day during the ICS reduction/withdrawal period was about twice as high for Placebo-treated subjects with a high Th2 level than for those with a normal Th2 level, for any of the significant markers. Similar results were obtained by logistic regression analysis with odds ratios larger than 2 when comparing the Th2 high subset of trial participants with the Th2 normal subset, for any of the four significant markers. This means that the odds of experiencing at least one asthma exacerbation in the complete ICS reduction/withdrawal period were more than twice as high for Th2 high subjects than for Th2 normal subjects.

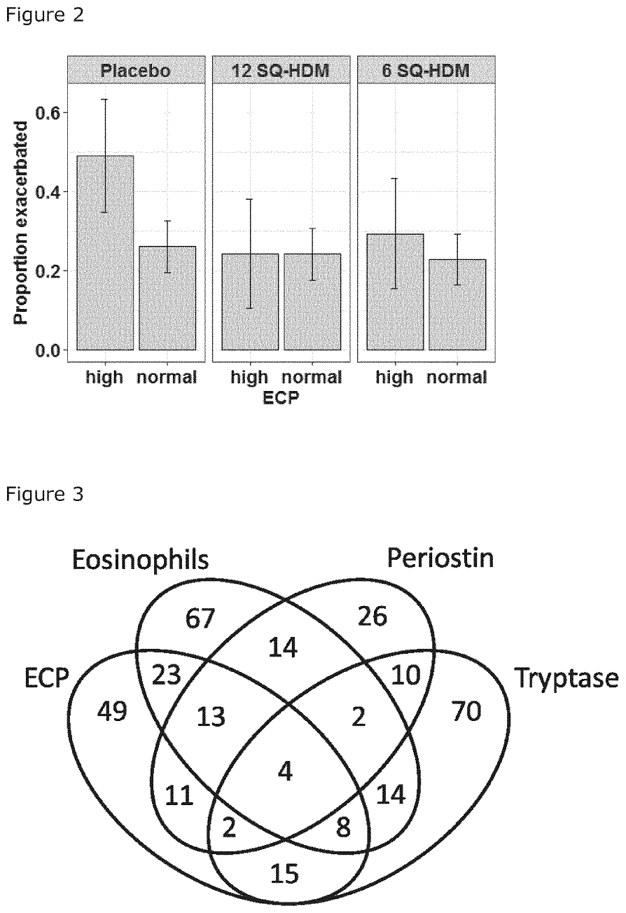

FIG. 2 shows an example of the proportion of patients experiencing an exacerbation in different treatment groups further divided into Th2 high and Th2 normal depending on the measured concentration of ECP. It can be seen that the proportion of Placebo treated subjects in the Th2 high group experiencing an exacerbation is about 50%, while in the Th2 high groups treated with either 12 SQ-HDM or 6 SQ-HDM, the proportion is reduced to a level between 20% to 30%.

TABLE 14
Hazard and Odds ratios for ECP

| ECP | Hazard Ratio | lwr | upr | p-value |
|---|---|---|---|---|
| high.Placebo - normal.Placebo | 2.09 | 1.26 | 3.47 | 0.004 |
| high.12SQ - high.Placebo | 0.43 | 0.20 | 0.93 | 0.032 |
| high.6SQ - high.Placebo | 0.52 | 0.26 | 1.05 | 0.069 |
| ECP | Odds Ratio | lwr | upr | p-value |
| high.Placebo - normal.Placebo | 2.72 | 1.40 | 5.31 | 0.003 |
| high.12SQ - high.Placebo | 0.34 | 0.13 | 0.86 | 0.023 |
| high.6SQ - high.Placebo | 0.43 | 0.18 | 1.04 | 0.062 |

TABLE 15
Hazard and Odds ratios for eosinophil count

| Eosinophil count | Hazard Ratio | lwr | upr | p-value |
|---|---|---|---|---|
| high.Placebo - normal.Placebo | 1.74 | 1.06 | 2.84 | 0.028 |
| high.12SQ - high.Placebo | 0.60 | 0.29 | 1.22 | 0.156 |
| high.6SQ - high.Placebo | 0.52 | 0.26 | 1.02 | 0.058 |
| Eosinophil count | Odds Ratio | lwr | upr | p-value |
| high.Placebo - normal.Placebo | 1.99 | 1.06 | 3.73 | 0.032 |
| high.12SQ - high.Placebo | 0.54 | 0.23 | 1.31 | 0.173 |
| high.6SQ - high.Placebo | 0.46 | 0.20 | 1.04 | 0.061 |

TABLE 16
Hazard and Odds ratios for periostin

| Periostin | Hazard Ratio | lwr | upr | p-value |
|---|---|---|---|---|
| high.Placebo - normal.Placebo | 2.13 | 1.22 | 3.72 | 0.008 |
| high.12SQ - high.Placebo | 0.36 | 0.15 | 0.85 | 0.021 |
| high.6SQ - high.Placebo | 0.30 | 0.12 | 0.76 | 0.011 |
| Periostin | Odds Ratio | lwr | upr | p-value |
| high.Placebo - normal.Placebo | 2.85 | 1.24 | 6.51 | 0.013 |
| high.12SQ - high.Placebo | 0.25 | 0.08 | 0.76 | 0.015 |
| high.6SQ - high.Placebo | 0.20 | 0.06 | 0.64 | 0.007 |

TABLE 17
Hazard and Odds ratios for tryptase

| Tryptase | Hazard Ratio | lwr | upr | p-value |
|---|---|---|---|---|
| high.Placebo - normal.Placebo | 1.88 | 1.13 | 3.11 | 0.015 |
| high.12SQ - high.Placebo | 0.46 | 0.21 | 0.99 | 0.048 |
| high.6SQ - high.Placebo | 0.53 | 0.26 | 1.09 | 0.084 |
| Tryptase | Odds Ratio | lwr | upr | p-value |
| high.Placebo - normal.Placebo | 2.36 | 1.23 | 4.54 | 0.010 |
| high.12SQ - high.Placebo | 0.39 | 0.15 | 1.00 | 0.050 |
| high.6SQ - high.Placebo | 0.46 | 0.19 | 1.13 | 0.089 |

TABLE 18
Hazard and Odds ratios for total IgE

| Total IgE | Hazard Ratio | lwr | upr | p-value |
|---|---|---|---|---|
| high.Placebo - normal.Placebo | 1.69 | 1.02 | 2.82 | 0.043 |
| high.12SQ - high.Placebo | 0.52 | 0.25 | 1.10 | 0.087 |
| high.6SQ - high.Placebo | 0.47 | 0.20 | 1.09 | 0.080 |
| Total IgE | Odds Ratio | lwr | upr | p-value |
| high.Placebo - normal.Placebo | 1.79 | 0.94 | 3.40 | 0.076 |
| high.12SQ - high.Placebo | 0.47 | 0.19 | 1.15 | 0.098 |
| high.6SQ - high.Placebo | 0.44 | 0.16 | 1.21 | 0.112 |

The levels of the Th2 markers ECP, blood eosinophils, periostin and tryptase were found only to be weakly correlated with each other, which means that they are not redundant in their effect on asthma exacerbation risk. FIG. 3 shows a Venn diagram with the numbers of subjects in each combination of Th2 high groups depending on the levels of one or more of the biomarkers quantified as mentioned above. Therefore, we combined these four markers in all possible combinations of 2, 3 or 4 markers into a Th2 meta score, resulting in 11 combinations (e.g. Eosinophils & ECP, ECP & Tryptase, ECP & Tryptase & Periostin, etc.). For each of the possible combinations of 2, 3 or 4 markers, we defined a trial participant as having a “Th2 high” status if one or more of the combined markers were high. See Table 19 demonstrating the percentage of subjects in each “Th2 high” group of combined Th2 markers compared to the total trial population. It can be seen from the numbers, that when a combination of two markers are used, the percentage of Th2 high patients in the total trial population is between 27% to 36% of the total trial population. When a combination of three markers are used, the percentage of Th2 high patients is between 39% to 45% of the total trial population, and when the combination of four markers is used, the percentage of Th2 high patients compared to the total trial population is 49%.

TABLE 19
Percentage of subjects in Th2 high subpopulations
compared to the total trial population

| | % of trial |
|---|---|
| Th2 high subpopulation | population |
| Eosinophils & ECP | 33% |
| Eosinophils & Tryptase | 36% |
| Eosinophils & Periostin | 29% |
| ECP & Tryptase | 33% |
| ECP & Periostin | 27% |
| Tryptase & Periostin | 28% |
| Eosinophils & ECP & Tryptase | 45% |
| Eosinophils & ECP & Periostin | 39% |
| Eosinophils & Tryptase & Periostin | 42% |
| ECP & Tryptase & Periostin | 39% |
| All four | 49% |

Each of these Th2 meta scores was then tested for its effect on exacerbation risk in the three treatment groups, and each of these Th2 meta scores resulted in a significant CoxPH model. These models resulted in large hazard ratios in the Placebo-treated group when comparing the risk to exacerbate for Th2 high individuals with Th2 normal individuals, where hazard ratios ranged from 1.97 (P value=0.005) when combining eosinophil count and ECP to 3.24 (P value<1e-05) when combining ECP, Tryptase and Periostin (see comparisons “high.Placebo-normal.Placebo” in Table 20, Table 21, Table 22, Table 23, Table 24, and Table 25).

In addition, we observe a large treatment effect in the Th2 high subpopulation for all of the combinations, with hazard ratios ranging between 0.37 (P value=0.0005 for ECP & Tryptase & Periostin) and 0.57 (P value=0.01 for Eosinophils & ECP & Tryptase & Periostin) when comparing the subpopulations treated with 12 SQ-HDM and 6 SQ-HDM, respectively, with the Placebo group (see comparisons “high.12SQ-high.Placebo” and “high.6SQ-high.Placebo” in Table 20, Table 21, Table 22, Table 23, Table 24, and Table 25). This means that treatment with either of the two doses 6 SQ-HDM or 12 SQ-HDM reduced the risk to exacerbate at any given day during the ICS reduction/withdrawal period by 43 to 63%, depending on the Th2 marker combination.

TABLE 20
Combinations with eosinophil count and one other Th2 marker

| Eosinophil count & ECP | Hazard Ratio | lwr | upr | p-value |
|---|---|---|---|---|
| high.Placebo - normal.Placebo | 1.97 | 1.23 | 3.15 | 0.005 |
| high.12SQ - high.Placebo | 0.54 | 0.30 | 0.96 | 0.037 |
| high.6SQ - high.Placebo | 0.56 | 0.32 | 0.97 | 0.038 |
| Eosinophils & ECP | Odds Ratio | lwr | upr | p-value |
| high.Placebo - normal.Placebo | 2.36 | 1.32 | 4.20 | 0.004 |
| high.12SQ - high.Placebo | 0.47 | 0.23 | 0.97 | 0.040 |
| high.6SQ - high.Placebo | 0.49 | 0.25 | 0.97 | 0.039 |
| Eosinophils & Tryptase | Hazard Ratio | lwr | upr | p-value |
| high.Placebo - normal.Placebo | 2.33 | 1.45 | 3.74 | 0.0005 |
| high.12SQ - high.Placebo | 0.53 | 0.30 | 0.92 | 0.024 |
| high.6SQ - high.Placebo | 0.55 | 0.33 | 0.92 | 0.023 |
| Eosinophils & Tryptase | Odds Ratio | lwr | upr | p-value |
| high.Placebo - normal.Placebo | 2.88 | 1.61 | 5.15 | 0.0003 |
| high.12SQ - high.Placebo | 0.48 | 0.24 | 0.94 | 0.032 |
| high.6SQ - high.Placebo | 0.49 | 0.26 | 0.91 | 0.025 |
| Eosinophils & Periostin | Hazard Ratio | lwr | upr | p-value |
| high.Placebo - normal.Placebo | 2.23 | 1.39 | 3.57 | 0.0008 |
| high.12SQ - high.Placebo | 0.56 | 0.31 | 1.02 | 0.056 |
| high.6SQ - high.Placebo | 0.46 | 0.26 | 0.83 | 0.010 |
| Eosinophils & Periostin | Odds Ratio | lwr | upr | p-value |
| high.Placebo - normal.Placebo | 2.75 | 1.52 | 5.00 | 0.0009 |
| high.12SQ - high.Placebo | 0.49 | 0.23 | 1.03 | 0.059 |
| high.6SQ - high.Placebo | 0.39 | 0.19 | 0.80 | 0.010 |

TABLE 21
Combinations of ECP with one other Th2 marker

| ECP & Tryptase | Hazard Ratio | lwr | upr | p-value |
|---|---|---|---|---|
| high.Placebo - normal.Placebo | 2.55 | 1.58 | 4.10 | 0.0001 |
| high.12SQ - high.Placebo | 0.39 | 0.21 | 0.72 | 0.002 |
| high.6SQ - high.Placebo | 0.54 | 0.32 | 0.92 | 0.024 |
| ECP & Tryptase | Odds Ratio | lwr | upr | p-value |
| high.Placebo - normal.Placebo | 3.34 | 1.86 | 5.99 | 0.00005 |
| high.12SQ - high.Placebo | 0.32 | 0.16 | 0.66 | 0.002 |
| high.6SQ - high.Placebo | 0.47 | 0.24 | 0.91 | 0.026 |
| ECP & Periostin | Hazard Ratio | lwr | upr | p-value |
| high.Placebo - normal.Placebo | 2.70 | 1.69 | 4.32 | 0.00003 |
| high.12SQ - high.Placebo | 0.40 | 0.22 | 0.76 | 0.005 |
| high.6SQ - high.Placebo | 0.46 | 0.25 | 0.83 | 0.011 |
| ECP & Periostin | Odds Ratio | lwr | upr | p-value |
| high.Placebo - normal.Placebo | 3.65 | 1.98 | 6.74 | 0.00003 |
| high.12SQ - high.Placebo | 0.31 | 0.14 | 0.68 | 0.003 |
| high.6SQ - high.Placebo | 0.37 | 0.17 | 0.78 | 0.009 |

TABLE 22
Combination of tryptase with periostin

| Tryptase & Periostin | Hazard Ratio | lwr | upr | p-value |
|---|---|---|---|---|
| high.Placebo - normal.Placebo | 2.43 | 1.52 | 3.88 | 0.0002 |
| high.12SQ - high.Placebo | 0.37 | 0.19 | 0.72 | 0.003 |
| high.6SQ - high.Placebo | 0.50 | 0.28 | 0.89 | 0.018 |
| Tryptase & Periostin | Odds Ratio | lwr | upr | p-value |
| high.Placebo - normal.Placebo | 3.16 | 1.74 | 5.72 | 0.0001 |
| high.12SQ - high.Placebo | 0.30 | 0.14 | 0.67 | 0.003 |
| high.6SQ - high.Placebo | 0.42 | 0.20 | 0.87 | 0.019 |

TABLE 23
Combinations of eosinophil count with two other markers

| Eosinophil count & ECP & | | | | |
|---|---|---|---|---|
| Tryptase | Hazard Ratio | lwr | upr | p-value |
| high.Placebo - normal.Placebo | 3.00 | 1.77 | 5.08 | 0.00004 |
| high.12SQ - high.Placebo | 0.47 | 0.28 | 0.78 | 0.004 |
| high.6SQ - high.Placebo | 0.56 | 0.35 | 0.89 | 0.014 |
| Eosinophil count & ECP & | | | | |
| Tryptase | Odds Ratio | lwr | upr | p-value |
| high.Placebo - normal.Placebo | 3.84 | 2.08 | 7.10 | 0.00002 |
| high.12SQ - high.Placebo | 0.41 | 0.22 | 0.76 | 0.005 |
| high.6SQ - high.Placebo | 0.50 | 0.28 | 0.88 | 0.016 |
| Eosinophil count & ECP & | | | | |
| Periostin | Hazard Ratio | lwr | upr | p-value |
| high.Placebo - normal.Placebo | 2.33 | 1.45 | 3.75 | 0.0005 |
| high.12SQ - high.Placebo | 0.53 | 0.32 | 0.90 | 0.019 |
| high.6SQ - high.Placebo | 0.53 | 0.32 | 0.88 | 0.014 |
| Eosinophil count & ECP & | | | | |
| Periostin | Odds Ratio | lwr | upr | p-value |
| high.Placebo - normal.Placebo | 2.88 | 1.61 | 5.15 | 0.0003 |
| high.12SQ - high.Placebo | 0.47 | 0.24 | 0.89 | 0.021 |
| high.6SQ - high.Placebo | 0.46 | 0.24 | 0.85 | 0.014 |
| Eosinophil count & Tryptase | | | | |
| & Periostin | Hazard Ratio | lwr | upr | p-value |
| high.Placebo - normal.Placebo | 2.55 | 1.56 | 4.15 | 0.0002 |
| high.12SQ - high.Placebo | 0.51 | 0.30 | 0.86 | 0.011 |
| high.6SQ - high.Placebo | 0.55 | 0.34 | 0.89 | 0.016 |
| Eosinophil count & Tryptase | | | | |
| & Periostin | Odds Ratio | lwr | upr | p-value |
| high.Placebo - normal.Placebo | 3.19 | 1.77 | 5.74 | 0.0001 |
| high.12SQ - high.Placebo | 0.45 | 0.24 | 0.85 | 0.014 |
| high.6SQ - high.Placebo | 0.49 | 0.27 | 0.88 | 0.018 |

TABLE 24
Combination of ECP with tryptase and periostin

| ECP & Tryptase & Periostin | Hazard Ratio | lwr | upr | p-value |
|---|---|---|---|---|
| high.Placebo - normal.Placebo | 3.24 | 1.95 | 5.36 | 0.000005 |
| high.12SQ - high.Placebo | 0.37 | 0.22 | 0.65 | 0.0005 |
| high.6SQ - high.Placebo | 0.54 | 0.33 | 0.88 | 0.013 |
| ECP & Tryptase & Periostin | Odds Ratio | lwr | upr | p-value |
| high.Placebo - normal.Placebo | 4.32 | 2.36 | 7.90 | 0.000002 |
| high.12SQ - high.Placebo | 0.30 | 0.16 | 0.59 | 0.0004 |
| high.6SQ - high.Placebo | 0.46 | 0.25 | 0.86 | 0.015 |

TABLE 25
Combination of four Th2 markers

| Eosinophils & ECP & Tryptase | | | | |
|---|---|---|---|---|
| & Periostin | Hazard Ratio | lwr | upr | p-value |
| high.Placebo - normal.Placebo | 3.21 | 1.86 | 5.55 | 0.00003 |
| high.12SQ - high.Placebo | 0.48 | 0.30 | 0.77 | 0.003 |
| high.6SQ - high.Placebo | 0.57 | 0.37 | 0.89 | 0.014 |
| Eosinophils & ECP & Tryptase | | | | |
| & Periostin | Odds Ratio | lwr | upr | p-value |
| high.Placebo - normal.Placebo | 4.13 | 2.20 | 7.75 | 0.00001 |
| high.12SQ - high.Placebo | 0.42 | 0.23 | 0.75 | 0.003 |
| high.6SQ - high.Placebo | 0.51 | 0.29 | 0.88 | 0.016 |

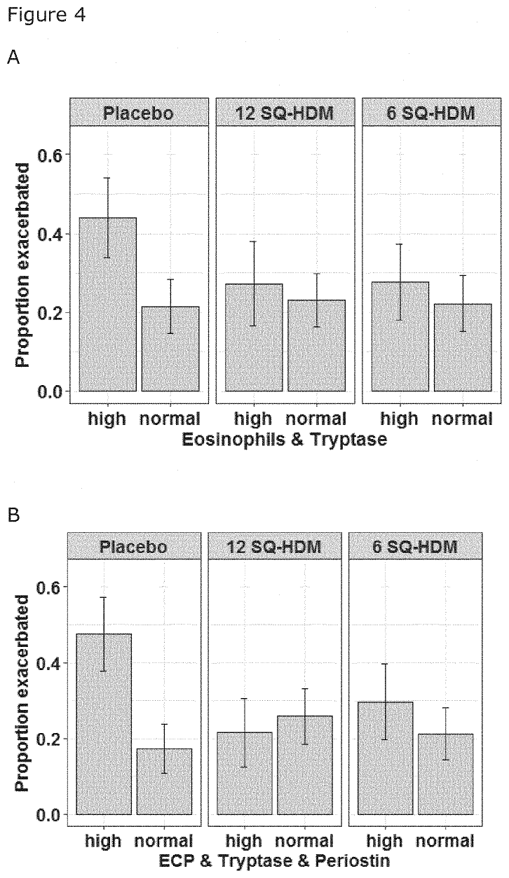

FIG. 4 shows an example of the proportion of patients experiencing an exacerbation in different treatment groups further divided into Th2 high and Th2 normal depending on the eosinophil count and the measured concentration of tryptase (FIG. 4A), or depending on the measured concentration of ECP, tryptase and periostin (FIG. 4B). It can be seen that the proportion of placebo treated subjects in the Th2 high group experiencing an exacerbation is about 45% to about 50%, while in the Th2 high groups treated with either 12 SQ-HDM or 6 SQ-HDM, the proportion is reduced to levels between 20-30%. Similar results demonstrating a marked reduction in proportion of subjects experiencing an exacerbation were seen for the other combinations of two or more of the four markers eosinophil count, ECP, tryptase and periostin.

Thus, the results demonstrate that the quantification of one or more Th2 markers in subjects suffering from allergic asthma can be used for selecting patients having a high probability of a beneficial treatment effect in that the risk of having an asthma exacerbation is reduced.

### Example 2

The present example discloses a clinical trial, wherein a fast dispersing tablet comprising grass pollen allergen extract for sublingual administration was tested for effects on prevention of asthma in children with a clinical history of grass pollen allergic rhinoconjunctivitis and no medical history of asthma.

The GRAZAX® Asthma Prevention (GAP) clinical trial was a randomized, double-blind, placebo-controlled trial, comprising 3 years of treatment and 2 years of follow-up and is described in detail in Valovirta et al 2017. A total of 812 children (5-12 years of age) with grass pollen allergy (positive skin prick test response to grass pollen allergen extract and specific IgE to Phleum pratense), and a clinically relevant history of grass pollen allergic rhinoconjunctivitis requiring allergy pharmacotherapy during two grass pollen seasons were included in the trial. Further inclusion criteria were no medical history of asthma and/or wheezing (including no signs of asthma within the last 2 years or since the fifth birthday).

The primary endpoint was time to onset of asthma measured in days from randomization. Asthma was defined as the fulfillment of 1 or more of the following 3 criteria, which were evaluated at each trial visit for each time period “since last visit”:

(1) At least 1 episode of wheeze, cough, shortness of breath or chest tightness, and a change in FEV1>12% after β₂-agonist administration;

(2) Wheezing with or without prolonged phase of forced exhalation observed at physical examination and an intake of asthma medication, which resulted in a clinically relevant effect;

(3) Wheezing with or without prolonged phase of forced exhalation observed at physical examination and a change in FEV1>12% after β₂-agonist administration.

According to the trial protocol, the onset of asthma was considered a binary event (asthma yes/no) that only could occur once per subject. Thus, the children were only classified as having asthma if the criteria were met at a single given visit, and clinical information from previous or subsequent visits was not taken into consideration when this classification was made. Children with frequent reporting of asthma symptoms but no observable reversible impairment of lung function at trial visits were not classified as having asthma. Asthma symptom and asthma medication status at end of trial (in the period from winter visit in year 5 to GPS visit in year 5) was a predefined secondary asthma endpoint. The proportion of children with asthma symptoms and/or asthma medication use during the course of the trial was further characterized post hoc and analyzed for the entire trial and the follow-up period. In addition, the number of children needed to treat (NNT) to prevent 1 additional child from having asthma symptoms and using asthma medication was analyzed post hoc for the 2-year follow-up period.

The primary efficacy analysis of time to onset of asthma as defined by the prespecified asthma diagnosis criteria showed no difference between SQ grass SLIT tablet and placebo.

However, of the 739 children not diagnosed with asthma according to the above protocol criteria, 147 children reported asthma symptoms or use of asthma medication during the 2-year follow-up period, and 66 of those children (41 children on placebo and 25 children on SQ grass SLIT tablet) reported both asthma symptoms and medication use during that period. The predefined efficacy analysis of asthma symptom and medication status at the end of the trial (assessed at GPS visit in year 5) showed that fewer children on SQ grass SLIT tablet treatment compared with placebo-treated children experienced asthma symptoms or used asthma medication; odds ratio (OR)=0.66; P<0.036, corresponding to a relative risk reduction of 29.4%.

The proportion of children experiencing asthma symptoms or using asthma medication was further characterized post hoc each year of the 5-year trial period. The OR for experiencing asthma symptoms or using asthma medication on SQ grass SLIT tablet versus placebo treatment was in favor of the SQ grass SLIT tablet each year, with statistical significance (P value<0.05) from year 2 onward the corresponding relative risk reductions ranged from 36.2% to 50.7%.

Also, grass allergic rhinoconjunctivitis symptoms were 22% to 30% reduced (P<0.005 for all 5 years). At the end of the trial, the use of allergic rhinoconjunctivitis pharmacotherapy was significantly less (27% relative difference to placebo, P<0.001).

To conclude, the treatment with allergen immunotherapy treatment using grass pollen allergen extract reduced the risk of experienced asthma symptoms and use of asthma medication remarkably compared to the placebo treatment, in children with no previous signs or medical history of asthma.

### Example 3

The present example relates to a genotyping study of subjects included in the clinical trial of Example 2. The genotyping and subsequent analysis described in Example 1 is performed on biological samples of participants in the clinical study described in Example 2.

### Example 4

The present example relates to a study of biomarkers related to Th2-mediated inflammation in subjects included in the clinical trial of Example 2. The quantification of Th2 biomarkers and subsequent analysis described in Example 1 is performed on biological samples of participants in the clinical study described in Example 2.

### Example 5

The present example describes an example of use of one particular SNP marker of the 17q12-21 region in relation to selection of a patient for treatment with allergen-specific immunotherapy. A skilled person will understand that other markers (i.e. SNP markers and/or Th2 markers, or a combination thereof) as disclosed herein could be used in the method below.

A patient suffering from asthma is tested positive for house dust mite-specific serum IgE, and genotyping of a biological sample comprising genomic DNA shows that the patient is a homozygote of the rs7216389 T effect allele. The patient is subsequently treated by daily sublingual administration of an allergen specific immunotherapy product comprising house dust mite allergen extract. During and after treatment the subject experiences a decreased risk (increased time between occurence) of moderate to severe asthma exacerbations, compared to before treatment with allergen-specific immunotherapy.

### Example 6

The present example describes an example of use of one particular SNP marker of the 17q12-21 region in relation to selection of a patient for treatment with allergen-specific immunotherapy. A skilled person will understand that other markers (i.e. SNP markers and/or Th2 markers, or a combination thereof) as disclosed herein could be used in the method below.

A patient who has previously been diagnosed with house dust mite allergy contacts a medical doctor due to experiencing asthma symptoms such as wheezing, shortness of breath, chest tightness, increased coughing, fatigue and nocturnal awakening. Genotyping of a biological sample comprising genomic DNA from the patient shows that the patient is a homozygote of the rs7216389 T effect allele. The patient is subsequently treated by daily sublingual administration an allergen specific immunotherapy product comprising house dust mite allergen extract. During and after treatment the subject experiences an improvement in asthma symptoms and decreased frequency of moderate to severe asthma exacerbations, compared to before treatment with allergen-specific immunotherapy.

### REFERENCES

Anonymous. Infinium Global Screening Array-24, document published on the the website:URL:https://www.illumina.com/content/dam/illumina-marketing/documents/products/datasheets/infinium-commercial-gsa-data-sheet-370-2016-016
Bel E H, Sousa A, Fleming L, Bush A, Chung K F, Versnel 3, Wagener A H, Wagers S S, Sterk P J, Compton C H; Unbiased Biomarkers for the Prediction of Respiratory Disease Outcome (U-BIOPRED) Consortium, Consensus Generation. Diagnosis and definition of severe refractory asthma: an international consensus statement from the Innovative Medicine Initiative (IMI). Thorax. 2011 October; 66(10):910-7. doi: 10.1136/thx.2010.153643. Epub 2010 Nov. 23.
Bisgaard H, Bønnelykke K, Sleiman P M, Brasholt M, Chawes B, Kreiner-Møller E, Stage M, Kim C, Tavendale R, Baty F, Pipper C B, Palmer C N, Hakonarsson H. Chromosome 17q21 gene variants are associated with asthma and exacerbations but not atopy in early childhood. Am J Respir Crit Care Med. 2009 Feb. 1; 179(3):179-85. doi: 10.1164/rccm.200809-14360C.
Blekic M, Kljaic Bukvic B, Aberle N, Marinho S, Hankinson J, Custovic A, Simpson A. 17q12-21 and asthma: interactions with early-life environmental exposures. Ann Allergy Asthma Immunol. 2013 May; 110(5):347-353.e2. doi: 10.1016/j.anai.2013.01.021.
Bønnelykke K, Sleiman P, Nielsen K, Kreiner-Møller E, Mercader J M, Belgrave D, den Dekker H T, Husby A, Sevelsted A, Faura-Tellez G, Mortensen L J, Paternoster L, Flaaten R, Mølgaard A, Smart D E, Thomsen P F, Rasmussen M A, Bonàs-Guarch S, Hoist C, Nohr E A, Yadav R, March M E, Blicher T, Lackie P M, Jaddoe V W, Simpson A, Holloway J W, Duijts L, Custovic A, Davies D E, Torrents D, Gupta R, Hollegaard M V, Hougaard D M, Hakonarson H, Bisgaard H. A genome-wide association study identifies CDHR3 as a susceptibility locus for early childhood asthma with severe exacerbations. Nat Genet. 2014 January; 46(1):51-5. doi: 10.1038/ng.2830. Epub 2013 Nov. 17.
Chiappori A, De Ferrari L, Folli C, Mauri P, Riccio A M, Canonica G W. Biomarkers and severe asthma: a critical appraisal. Clin Mol Allergy. 2015 Oct. 1; 13:20. doi: 10.1186/s12948-015-0027-7. eCollection 2015. Review.
Dahl R. Systemic side effects of inhaled corticosteroids in patients with asthma. Respir Med. 2006 August; 100(8):1307-17. Epub 2006 Jan. 18.
FDA (Food, Drug Administration) HHS. International Conference on harmonisation; guidance on E15 pharmacogenomics definitions and sample coding; availability. Notice. Fed Regist 2008; 73:19074-19076.
Ferreira M A, Matheson M C, Tang C S, Granell R, Ang W, Hui J, Kiefer A K, Duffy, D L, Baltic S, Danoy P, Bui M, Price L, Sly P D, Eriksson N, Madden P A, Abramson, M J, Holt P G, Heath A C, Hunter M, Musk B, Robertson C F, Le Souef P, Montgomery G W, Henderson A J, Tung J Y, Dharmage S C, Brown M A, James A, Thompson P J, Pennell C, Martin N G, Evans D M, Hinds D A, Hopper J L; Australian Asthma Genetics Consortium Collaborators. Genome-wide association analysis identifies 11 risk variants associated with the asthma with hay fever phenotype. J Allergy Clin Immunol. 2014 June; 133(6):1564-71. doi: 10.1016/j.jaci.2013.10.030. Epub 2013 Dec. 31.
Galanter J, Choudhry S, Eng C, Nazario S, Rodriguez-Santana J R, et al. (2008) ORMDL3 gene is associated with asthma in three ethnically diverse populations. Am J Respir Crit Care Med 177: 1194-1200.
Hill W G, Robertson A. Linkage disequilibrium in finite populations. Theoretical and Applied Genetics. 1968; 38:226-231. PMID: 24442307
Katial R K, Bensch G W, Busse W W, Chipps B E, Denson J L, Gerber A N, Jacobs J S, Kraft M, Martin R J, Nair P, Wechsler M E. Changing Paradigms in the Treatment of Severe Asthma: The Role of Biologic Therapies. J Allergy Clin Immunol Pract. 2017 March-April; 5(2S):S1-S14. doi: 10.1016/j.jaip.2016.11.029.
Loss G J, Depner M, Hose A J, Genuneit J, Karvonen A M, Hyvarinen A, et al. The early development of wheeze. Environmental determinants and genetic susceptibility at 17q21. Am Respir Crit Care Med 2016; 193:889-97.
Madore A M, Tremblay K, Hudson T J, Laprise C (2008) Replication of an association between 17q21 SNPs and asthma in a French-Canadian familial collection. Hum Genet 123: 93-95.
Moffatt M F, Kabesch M, Liang L, Dixon A L, Strachan D, Heath S, Depner M, von Berg A, Bufe A, Rietschel E, Heinzmann A, Simma B, Frischer T, Willis-Owen S A, Wong K C, Illig T, Vogelberg C, Weiland S K, von Mutius E, Abecasis G R, Farrall M, Gut I G, Lathrop G M, Cookson W O. Genetic variants regulating ORMDL3 expression contribute to the risk of childhood asthma. Nature. 2007 Jul. 26; 448(7152):470-3.
Moffatt M F, Gut I G, Demenais F, Strachan D P, Bouzigon E, Heath S, von Mutius E, Farrall M, Lathrop M, Cookson W O C M; GABRIEL Consortium. A large-scale, consortium-based genomewide association study of asthma. N Engl J Med. 2010 Sep. 23; 363(13):1211-1221. doi: 10.1056/NEJMoa0906312.
Ober C, Yao T C. The genetics of asthma and allergic disease: a 21st century perspective. Immunol Rev. 2011 July; 242(1):10-30. doi:10.1111/j.1600-065X.2011.01029.x. Review.
Possa S S, Leick E A, Prado C M, Martins M A, Tiberio I F. Eosinophilic inflammation in allergic asthma. Front Pharmacol 2013; 4:46.
Purcell S, Neale B, Todd-Brown K, Thomas L, Ferreira M A R, Bender D, Maller J, Sklar P, de Bakker P I W, Daly M J & Sham P C. PLINK: a toolset for whole-genome association and population-based linkage analysis. Am J Hum Genet. 2007 September; 81(3):559-75.
Shamji M H, Kappen J H, Akdis M, Jensen-Jarolim E, Knol E F, Kleine-Tebbe J, Bohle B, Chaker A M, Till S J, Valenta R, Poulsen L K, Calderon M A, Demoly P, Pfaar O Jacobsen L, Durham S R, Schmidt-Weber C B. Biomarkers for monitoring clinical efficacy of allergen immunotherapy for allergic rhinoconjunctivitis and allergic asthma: an EAACI Position Paper. Allergy. 2017 August; 72(8):1156-1173. doi: 10.1111/a11.13138. Epub 2017 Apr. 6.
Stein M M, Thompson E E, Schoettler N, Helling B A, Magnaye K M, Stanhope C, Igartua C, Morin A, Washington C 3rd, Nicolae D, Bønnelykke K, Ober C. A decade of research on the 17q12-21 asthma locus: Piecing together the puzzle. J Allergy Clin Immunol. 2018 Jan. 4. pii: S0091-6749(17)32943-3. doi: 10.1016/j.jaci.2017.12.974.
Stokholm J, Chawes B L, Vissing N, Bønnelykke K, Bisgaard H. Cat exposure in early life decreases asthma risk from the 17q21 high-risk variant. J Allergy Clin Immunol. 2017 Oct. 14. pii: S0091-6749(17)31439-2. doi:10.1016/j.jaci.2017.07.044.
Tamm M, Richards D H, Beghe B, Fabbri L. Inhaled corticosteroid and long-acting beta2-agonist pharmacological profiles: effective asthma therapy in practice. Respir Med 2012; 106 Suppl 1:S9-19.
Virchow J C, Backer V, Kuna P, Prieto L, Nolte H, Villesen H H, Ljørring C, Riis B, de Blay F. Efficacy of a House Dust Mite Sublingual Allergen Immunotherapy Tablet in Adults With Allergic Asthma: A Randomized Clinical Trial. JAMA. 2016 Apr. 26; 315(16):1715-25. doi: 10.1001/jama.2016.3964.
Valovirta E, Petersen T H, Piotrowska T, Laursen M K, Andersen J S, Sorensen H F, Klink R; GAP investigators. Results from the 5-year SQ grass sublingual immunotherapy tablet asthma prevention (GAP) trial in children with grass pollen allergy. J Allergy Clin Immunol. 2018 February; 141(2):529-538.e13. Epub 2017 Jul. 6.
Woodruff P G, Modrek B, Choy D F, et al. T-helper type 2-driven inflammation defines major subphenotypes of asthma. Am J Respir Crit Care Med. 2009; 180(5):388-395.
Zissler U M, Esser-von Bieren J, Jakwerth C A, Chaker A M, Schmidt-Weber C B. Current and future biomarkers in allergic asthma. Allergy. 2016 April; 71(4):475-94. doi: 10.1111/a11.12828. Epub 2016 Jan. 18. Review.

1. A method for selecting a subject for treatment of an atopic disease with an immunotherapy product comprising one or more house dust mite allergens, the method comprising:
a. Detecting whether a biological sample obtained from said subject has one or more alleles of SNPs selected from the group consisting of rs7216389 (SEQ ID NO: 10), rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), and rs2290400 (SEQ ID NO: 2), or a complementary SNP thereof; and optionally
b. Selecting said subject for said treatment if one or more SNPs of step a) are detected.

2. The method according to claim 1, wherein said subject is selected for treatment with said immunotherapy product if said subject is a homozygote with respect to one or more SNPs selected from the group consisting of rs7216389 (SEQ ID NO: 10), rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), and rs2290400 (SEQ ID NO: 2).

3. (canceled)

4. The method according to claim 1, wherein the treatment of an atopic disease comprises preventing, reducing risk of, treatment, ameliorating symptoms and signs of asthma.

5. The method according to claim 4, wherein signs of asthma are asthma exacerbations.

6. The method according to claim 1, wherein the atopic disease is allergic asthma.

7. The method according to claim 1, wherein the atopic disease is causes caused by exposure to house dust mites.

8. The method according to claim 1, wherein said immunotherapy product is for providing allergen-specific immunotherapy.

9. A method for predicting response to treatment of an atopic disease with an immunotherapy product comprising one or more house dust mite allergens, the method comprising:
a. Detecting whether a biological sample obtained from said subject has one or more alleles of SNPs selected from the group consisting of rs7216389 (SEQ ID NO: 10), rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), and rs2290400 (SEQ ID NO: 2), or a complementary SNP thereof; and optionally
b. Predicting a beneficial response to treatment with treatment if one or more SNPs of step a) are detected.

10. The method according to claim 9, wherein said subject is predicted to have a beneficial response to treatment using said immunotherapy product if said subject is a homozygote with respect to one or more SNPs selected from the group consisting of rs7216389 (SEQ ID NO: 10), rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), and rs2290400 (SEQ ID NO: 2).

11. (canceled)

12. The method according to claim 9, wherein the treatment of an atopic disease comprises preventing, reducing risk of, treatment, ameliorating symptoms and signs of asthma.

13. The method according to claim 12, wherein signs of asthma are asthma exacerbations.

14. The method according to claim 9, wherein the atopic disease is allergic asthma.

15. The method according to claim 9, wherein the atopic disease is caused by exposure to house dust mites.

16. The method according to claim 9, wherein said immunotherapy product is for providing allergen-specific immunotherapy.

17. An immunotherapy product comprising one or more house dust mite allergens for use in the treatment of an atopic disease in a selected subject, wherein the subject is selected by a method comprising:
a. Detecting whether a biological sample obtained from said subject has one or more alleles of SNPs selected from the group consisting of rs7216389 (SEQ ID NO: 10), rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), and rs2290400 (SEQ ID NO: 2), or a complementary SNP thereof; and optionally
b. Selecting said subject for said treatment with said immunotherapy product if one or more SNPs of step a) are detected.

18. The product for use according to claim 17, wherein said subject is selected for treatment using said immunotherapy product if said subject is a homozygote with respect to one or more SNPs selected from the group consisting of rs7216389 (SEQ ID NO: 10), rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), and rs2290400 (SEQ ID NO: 2).

19. (canceled)

20. The product for use according to claim 17, wherein the treatment of an atopic disease comprises preventing, reducing risk of, treatment, ameliorating symptoms and signs of asthma.

21. The product for use according to claim 20, wherein signs of asthma are asthma exacerbations.

22. The product for use according to claim 17, wherein the atopic disease is allergic asthma.

23. The product for use according to claim 17, wherein the atopic disease is caused by exposure to house dust mites.

24. The product for use according to claim 17, wherein said immunotherapy product is for providing allergen-specific immunotherapy.

25. A method of treating an atopic disease in a subject in need thereof, said method comprising:
a. Obtaining and/or providing a biological sample comprising nucleic acids from said subject; and
b. Detecting whether the biological sample has one or more alleles of SNPs selected from the group consisting of rs7216389 (SEQ ID NO: 10), rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), and rs2290400 (SEQ ID NO: 2), or a complementary SNP thereof; and optionally
c. Identifying or selecting said subject for treatment with an immunotherapy product comprising one or more house dust mite allergens if one or more SNPs of section b) are identified in the biological sample; and optionally
d. Administering said immunotherapy product to said subject identified or selected according to step c).

26. The method according to claim 25, wherein said subject is selected for treatment using said immunotherapy product if said subject is a homozygote with respect to one or more SNPs selected from the group consisting of rs7216389 (SEQ ID NO: 10), rs907092 (SEQ ID NO: 1), rs9303277 (SEQ ID NO: 13), rs8069176 (SEQ ID NO: 11), rs2305480 (SEQ ID NO: 3), rs11078927 (SEQ ID NO: 14), and rs2290400 (SEQ ID NO: 2).

27. (canceled)

28. The method according to claim 25, wherein the treatment of an atopic disease comprises preventing, reducing risk of, treatment, ameliorating symptoms and signs of asthma.

29. The method according to claim 28, wherein signs of asthma are asthma exacerbations.

30. The method according to claim 25, wherein the atopic disease is allergic asthma.

31. The method according to claim 25, wherein the atopic disease is caused by exposure to house dust mites.

32. The method according to claim 25, wherein said immunotherapy product is for providing allergen-specific immunotherapy.


SEQUENCE LISTING (35 sequences)

SEQ ID NO: 1 - DNA, 61 bp, Homo sapiens
cacctccccttccttgttgatcactttgacggagtctcttgggcagatgggcgggggcttg

SEQ ID NO: 2 - DNA, 61 bp, Homo sapiens
atctcagggccttacctcccactgactcttttccctctctgtgagccagctcctattgaag

SEQ ID NO: 3 - DNA, 61 bp, Homo sapiens
taaaaaggctgcttaggagaggcttgtctgggtcctccatgtgtagctccccggaaatcag

SEQ ID NO: 4 - DNA, 61 bp, Homo sapiens
aacaaaacgttccaagatgatccctgcccccacgtcttcaaagccggaactgacaccttcc

SEQ ID NO: 5 - DNA, 61 bp, Homo sapiens
ctctgtgaggtcagtgtccctggtgtggaaaactgtggaccagaaccatgcattgagggac

SEQ ID NO: 6 - DNA, 61 bp, Homo sapiens
tgtaccagaccctgtgctaagcaagggggtatggagaggagagggtgacaagaatattgga

SEQ ID NO: 7 - DNA, 61 bp, Homo sapiens
cccgggccctggccagacagctaaaccctcaaggggacctgacaccacttgacagcctcat

SEQ ID NO: 8 - DNA, 61 bp, Homo sapiens
agccccagggagagaattctcactcagcacgacccctgctggactgtgtctcactgaaggc

SEQ ID NO: 9 - DNA, 61 bp, Homo sapiens
aagtgaggcaaccctggaaagtcacaaacatgcatggactcggccctgattgatcaggcac

SEQ ID NO: 10 - DNA, 61 bp, Homo sapiens
gtttatattattatttttctacggtgatttggtttgctgatttgtttttctgttttttcat

SEQ ID NO: 11 - DNA, 61 bp, Homo sapiens
gtctcactgaaggcctaatgtgggacatctactcttaagctcttcccatcaggagggaaag

SEQ ID NO: 12 - DNA, 61 bp, Homo sapiens
ctgtgttcagtgtcatgcgatgaattaaaacagggctgtgttaaaatagtgactgaattgt

SEQ ID NO: 13 - DNA, 61 bp, Homo sapiens
tagtgtctttgcccattctgaaatgacgaacgggaagcccagcagctcactcaccttctgg

SEQ ID NO: 14 - DNA, 61 bp, Homo sapiens
ggattttgttttgcccctgaaatgaatatctaaaccagcaccaaaaagggaggaaaaaaca

SEQ ID NO: 15 - DNA, 61 bp, Homo sapiens
ccgaaaacttctgctgccatagctggcaggcggctgcttagtgctgggccctgatgtggtt

SEQ ID NO: 16 - DNA, 61 bp, Homo sapiens
cttgtagttacttacattagcccccagatgcagtgaaaccatcaagtataaattattcaaa

SEQ ID NO: 17 - DNA, 61 bp, Homo sapiens
gggaatgctaatagcctcaataacggctcaagagacttgtgaaagatacaatttaggagaa

SEQ ID NO: 18 - DNA, 61 bp, Homo sapiens
gtgaacagactttacacctgaaaatttcccgttagaacagtaattctttaatttttaggag

SEQ ID NO: 19 - DNA, 61 bp, Homo sapiens
caagttgtttcaatgttgtttaaggtaagatttgataactttggatttgtttgaagtttcg

SEQ ID NO: 20 - DNA, 61 bp, Homo sapiens
tttgtaaaagatccttttatacataaacaacgtgtctaaactatgaagcaaattacacaat

SEQ ID NO: 21 - DNA, 61 bp, Homo sapiens
aatttataactaaatatgttgtaattttgtcttctgacaagctaaattagcattatatagt

SEQ ID NO: 22 - DNA, 61 bp, Homo sapiens
atagatttggagttagaagtatttcatctcttgagtattagcaattattcattaaaaagaa

SEQ ID NO: 23 - DNA, 61 bp, Homo sapiens
gagtcgtcccggcctctggcgttctactcatgtgctgacctctttgtcctgcagcagtttt

SEQ ID NO: 24 - DNA, 61 bp, Homo sapiens
atctctctttcttttccccacagctacacacagagactgaggacaccctggacaacccagc

SEQ ID NO: 25 - DNA, 61 bp, Homo sapiens
gagaactccagctggtaactaaagtggactatgaaacaacccccatctatattctcagaat

SEQ ID NO: 26 - DNA, 61 bp, Homo sapiens
gcataactgaggaaacataacattttgatttatagttaatggtgattgggaacggtcattt

SEQ ID NO: 27 - DNA, 61 bp, Homo sapiens
tacatggaagaagaatgcatcaactgaaaatctattcctttgagaggaccaataaaaacaa

SEQ ID NO: 28 - DNA, 61 bp, Homo sapiens
aaaagatataaataagaataagaggtcatgctggtgtcttcatgagaaaagattgggaatt

SEQ ID NO: 29 - DNA, 61 bp, Homo sapiens
aacagtagttagctactttttaatagttacgagagcattggccaaggcagggaatctgtat

SEQ ID NO: 30 - DNA, 61 bp, Homo sapiens
accgagaatcaaaccgcaatctcggcagcacagggaatgagtaaaatacccatagtcgagg

SEQ ID NO: 31 - DNA, 61 bp, Homo sapiens
ggtcttccccacacttgtgaaatcacaggcgggcaacataatgacaggaagtcagctgtcg

SEQ ID NO: 32 - DNA, 61 bp, Homo sapiens
gccctccttagagaggggcctcaagggatgcccctggtctagacagaatgttccctactcc

SEQ ID NO: 33 - DNA, 61 bp, Homo sapiens
ctcagtgtggctcccaaggccctggagaccgtgcaggagaggtgagagtgggcgggactga

SEQ ID NO: 34 - DNA, 61 bp, Homo sapiens
attcccagtgcttatcatcacctgacctacgaaatatatggtttggtttctttactcattt

SEQ ID NO: 35 - DNA, 61 bp, Homo sapiens
agagagatatagaaactgacagacagagactggaaggcacagtcagaagcagaagtaagac